(19) United States
(12) Patent Application Publication
(10) Pub. No.: US 2011/0166124 A1
(43) Pub. Date: Jul. 7, 2011

(54) TRICYCLIC SPIROCYCLE DERIVATIVES AND METHODS OF USE

(76) Inventors: Kevin D. McCormick, Basking Ridge, NJ (US); Robert G. Aslanian, Rockaway, NJ (US); Pietro Mangiaracina, Monsey, NY (US); Michael Y. Berlin, Flemington, NJ (US); Manuel De Lera Ruiz, Branchburg, NJ (US); Christopher W. Boyce, Flemington, NJ (US); Jianhua Chao, San Diego, CA (US); Pauline C. Ting, New Providence, NJ (US); Junying Zheng, New Providence, NJ (US); Stuart B. Rosenblum, West Orange, NJ (US)

(21) Appl. No.: 13/054,806

(22) PCT Filed: Jul. 21, 2009
(86) PCT No.: PCT/US09/51267

§ 371 Date: Mar. 23, 2011

Related U.S. Application Data
(60) Provisional application No. 61/083,054, filed on Jul. 23, 2008.

(51) Int. Cl. C07D 471/10; C07D 491/107; C07D 495/10; A61K 31/438; A61K 31/501; A61K 31/553; C07D 498/10; A61P 37/08; A61P 9/00; A61P 29/00; A61P 1/00; A61P 27/00; A61P 3/00; A61P 3/06; A61P 3/10

(52) U.S. Cl. 514/210.21; 546/17; 514/278; 514/252.04; 544/230; 514/211.1

(57) ABSTRACT

The present invention relates to novel Tricyclic Spirocycle Derivatives, pharmaceutical compositions comprising the Tricyclic Spirocycle Derivatives and the use of these compounds for treating or preventing allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or aired fasting glucose.

### FIELD OF THE INVENTION

The present invention relates to novel Tricyclic Spirocycle Derivatives, pharmaceutical compositions comprising the Tricyclic Spirocycle Derivatives and the use of these compounds for treating or preventing allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose.

### BACKGROUND OF THE INVENTION

The histamine receptors, H₁, H₂ and H₃ are well-identified forms. The H₁ receptors are those that mediate the response antagonized by conventional antihistamines. H₁ receptors are present, for example, in the ileum, the skin, and the bronchial smooth muscle of humans and other mammals. Through H₂ receptor-mediated responses, histamine stimulates gastric acid secretion in mammals and the chronotropic effect in isolated mammalian atria.

H₃ receptor sites are found on sympathetic nerves, where they modulate sympathetic neurotransmission and attenuate a variety of end organ responses under control of the sympathetic nervous system. Specifically, H₃ receptor activation by histamine attenuates norepinephrine outflow to resistance and capacitance vessels, causing vasodilation.

Imidazole H₃ receptor antagonists are well known in the art. More recently, non-imidazole H₃ receptor antagonists have been disclosed in U.S. Pat. Nos. 6,720,328 and 6,849,621.

U.S. Pat. No. 5,869,479 discloses compositions for the treatment of the symptoms of allergic rhinitis using a combination of at least one histamine. H₁ receptor antagonist and at least one histamine H₃ receptor antagonist.

Diabetes refers to a disease process derived from multiple causative factors and is characterized by elevated levels of plasma glucose, or hyperglycemia in the fasting state or after administration of glucose during an oral glucose tolerance test. Persistent or uncontrolled hyperglycemia is associated with increased and premature morbidity and mortality. Abnormal glucose homeostasis is associated with alterations of the lipid, lipoprotein and apolipoprotein metabolism and other metabolic and hemodynamic disease. As such, the diabetic patient is at especially increased risk of macrovascular and microvascular complications, including coronary heart disease, stroke, peripheral vascular disease, hypertension, nephropathy, neuropathy, and retinopathy. Accordingly, therapeutic control of glucose homeostasis, lipid metabolism and hypertension are critically important in the clinical management and treatment of diabetes mellitus.

There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin dependent diabetes mellitus (NIDDM), patients often have plasma insulin levels that are the same or even elevated compared to nondiabetic subjects; however, these patients have developed a resistance to the insulin stimulating effect on glucose and lipid metabolism in the main insulin-sensitive tissue (muscle, liver and adipose tissue), and the plasma insulin levels, while elevated, are insufficient to overcome the pronounced insulin resistance.

Insulin resistance is not associated with a diminished number of insulin receptors but rather to a post-insulin receptor binding defect that is not well understood. This resistance to insulin responsiveness results in insufficient insulin activation of glucose uptake, oxidation and storage in muscle, and inadequate insulin repression of lipolysis in adipose tissue and of glucose production and secretion in the liver.

The available treatments for type 2 diabetes, which have not changed substantially in many years, have recognized limitations. While physical exercise and reductions in dietary intake of calories will dramatically improve the diabetic condition, compliance with this treatment is very poor because of well-entrenched sedentary lifestyles and excess food consumption, especially of foods containing high amounts of saturated fat. Increasing the plasma level of insulin by administration of sulfonylureas (e.g. tolbutamide and glipizide) or meglitinide, which stimulate the pancreatic [beta]-cells to secrete more insulin, and/or by injection of insulin when sulfonylureas or meglitinide become ineffective, can result in insulin concentrations high enough to stimulate the very insulin-resistant tissues. However, dangerously low levels of plasma glucose can result from administration of insulin or insulin secretagogues (sulfonylureas or meglitinide), and an increased level of insulin resistance due to the even higher plasma insulin levels can occur. The biguanides are a class of agents that can increase insulin sensitivity and bring about some degree of correction of hyperglycemia. However, the biguanides can induce lactic acidosis and nausea/diarrhea.

The glitazones (i.e. 5-benzylthiazolidine-2,4-diones) are a separate class of compounds with potential for the treatment of type 2 diabetes. These agents increase insulin sensitivity in muscle, liver and adipose tissue in several animal models of type 2 diabetes, resulting in partial or complete correction of the elevated plasma levels of glucose without occurrence of hypoglycemia. The glitazones that are currently marketed are agonists of the peroxisome proliferator activated receptor (PPAR), primarily the PPAR-gamma subtype. PPAR-gamma agonism is generally believed to be responsible for the improved insulin sensititization that is observed with the glitazones. Newer PPAR agonists that are being tested for treatment of Type 2 diabetes are agonists of the alpha, gamma or delta subtype, or a combination of these, and in many cases are chemically different from the glitazones (i.e., they are not thiazolidinediones). Serious side effects (e.g. liver toxicity) have been noted in some patients treated with glitazone drugs, such as troglitazone.

Additional methods of treating the disease are currently under investigation. New biochemical approaches include treatment with alpha-glucosidase inhibitors (e.g. acarbose) and protein tyrosine phosphatase-1B (PTP-1B) inhibitors.

Compounds that are inhibitors of the dipeptidyl peptidase-IV (DPP-IV) enzyme are also under investigation as drugs that may be useful in the treatment of diabetes, and particularly type 2 diabetes.

Despite a widening body of knowledge concerning the treatment of diabetes, there remains a need in the art for small-molecule drugs with increased safety profiles and/or improved efficacy that are useful for the treatment of diabetes and related metabolic diseases. The present invention addresses that need.

### SUMMARY OF THE INVENTION

In one aspect, the present invention provides Tricyclic Spirocycle Derivatives of Formula (I):

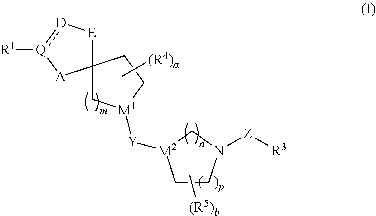

and pharmaceutically acceptable salts, solvates, esters and prodrugs thereof, wherein the dotted line represents an optional and additional bond, and wherein:

A is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)—, —C(═NH)N(R⁸)— or —N(R⁸)— or —C(R²¹)═N—;

D is —C(R²)— or —N— when the optional and additional bond is present, and D is —C(R²)₂— or —N(R²)— when the optional and additional bond is absent, such that when D is N and optional and additional bond is present, E is either —OC(R²¹)— or —O—;

E is a bond, —CH(R²¹)CH(R⁶)—, —CH(R²¹)C(O)—, —CH(R²¹)C(═N—OR⁹)—, —CH(R²¹)—, —C(O)CH(R⁶)—, —C(O)—, —C(═N—OR⁹)CH(R⁶)—, —C(═N—OR⁹)—, —C(═NH)N(R⁸)— —OCH(R²¹)— or —O— when D is —N—; and E is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —N(R⁸)CH(R⁶)CH(R²¹)—, N(R⁸)C(O)CH(R²¹)—, —N(R⁸)C(═N—OR⁹)CH(R²¹)—, —CH(R²)N(R⁸)—, —N(R⁸)C(O)—, —C(O)N(R⁸)—, —N(R⁸)C(═N—OR⁹)—, —C(═N—OR⁹)N(R⁸)— or —N(R⁸)— when D is other than —N—;

M¹ is —CH— or —N—;

M² is —CH—, —CF— or —N—;

Q is —C— when the optional and additional bond is present, and Q is —CH— when the optional and additional bond is absent;

Y is alkylene, -alkylene-C(O)—, —C(O)-alkylene-, —C(O)—, —C(S)—, —SO₂— or —O—;

Z is a bond, alkylene, alkenylene or -(alkylene)ᵥ-cycloalkylene-(alkylene)ᵥ-;

R¹ is H, aryl, -alkylene-aryl, heteroaryl, -alkylene-heteroaryl, alkyl, cycloalkyl or heterocycloalkyl, any of which can be optionally substituted with up to 4 substituents, which can be the same or different, and are selected from halo, —OH, alkyl, haloalkyl, —O-alkyl, —O-haloalkyl, —NO₂, —C(O)₇R¹², —N(R¹²)₂, —C(O)N(R¹²)₂, —NHC(O)R¹², —NHSO₂R¹², —SO₂N(R¹²)₂ and —CN, or R¹ and R², together with Q and D, combine to form an aryl, hetroaryl, cycloalkyl or heterocycloalkyl ring, any of which can be optionally substituted with up to 4 substituents, which can be the same or different, and are selected from halo, —OH, alkyl, haloalkyl, —O-alkyl, —O-haloalkyl, —NO₂, —CO₂R¹², —N(R¹²)₂, —C(O)N(R¹²)₂, —NHC(O)R¹², —NHSO₂R¹², —SO₂N(R¹²)₂ and —CN;

each occurrence of R² is independently H, aryl, -alkylene-aryl, heteroaryl, -alkylene-heteroaryl, alkyl, cycloalkyl or heterocycloalkyl, any of which can be optionally substituted with up to 4 substituents, which can be the same or different, and are selected from halo, —OH, alkyl, haloalkyl, —O-alkyl, —O-haloalkyl, —NO₂, —C(O)₂R¹², —N(R¹²)₂, —C(O)N(R¹²)₂, —NHC(O)R¹², —NHSO₂R¹², —SO₂N(R¹²)₂ and —CN;

R³ is H, alkyl, R²²-aryl, R²²-cycloalkyl, R²²-heterocycloalkyl or R²²-heteroaryl;

R⁴ and R⁵ are each independently halo, alkyl, —OH, —O-alkyl, haloalkyl or —CN;

each occurrence of R⁶ is independently H, halo, alkyl, haloalkyl, aryl, heteroaryl, cycloalkyl, heterocycloalkyl, —OH, —N(R¹³)₂, —NHC(O)R¹⁴, —NHC(O)₂R¹⁴, —NHC(O)NR¹⁴ or —NHSO₂R¹⁴;

R⁸ is H, alkyl, haloalkyl, aryl, cycloalkyl, heterocycloalkyl, heteroaryl, -alkylene-aryl, -alkylene-heteroaryl, —C(O)₂R¹⁴, —C(O)NR¹⁴ or —S(O)₂R¹⁴, wherein an aryl group can be optionally substituted with one or more alkyl groups, which can be the same or different;

R⁹ is H, alkyl, haloalkyl, aryl or heteroaryl;

each occurrence of R¹² is independently H, alkyl, aryl or heteroaryl;

each occurrence of R¹³ is hydrogen or alkyl;

each occurrence of R¹⁴ is independently alkyl, haloalkyl, aryl or heteroaryl;

each occurrence of R²¹ is independently H, alkyl, haloalkyl, aryl, heteroaryl, cycloalkyl or heterocycloalkyl;

each occurrence of R²² represents from 1 to 4 substituents, each independently selected from H, alkyl, haloalkyl, aryl, heteroaryl, cycloalkyl and heterocycloalkyl, wherein an aryl, heteroaryl, cycloalkyl or heterocycloalkyl group can be optionally and independently substituted with from 1 to 4 groups, each independently selected from alkyl, halo, —CN, —NO₂, alkyl, —N(R²¹)₂, —C(O)OR²¹, —C(O)N(R²¹)₂, —NHC(O)R²¹, —S(O)_{q}R²¹ or —OR²¹;

R²⁵ is selected from the group consisting of H and alkyl;

a is 0, 1 or 2;

b is 0, 1 or 2;

m is an integer ranging from 1 to 3;

n is 1 or 2, such that when M² is N, then n is 2;

p is 1 or 2;

each occurrence of q is 0, 1 or 2; and

each occurrence of v is 0 or 1.

The Compounds of Formula (I) and pharmaceutically acceptable salts, solvates, prodrugs and esters thereof can be useful for treating or preventing allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose (each being a “Condition”) in a patient.

Also provided by the invention are methods for treating or preventing Condition in a patient, comprising administering to the patient an effective amount of one or more compounds of Formula (I).

In addition, the present invention provides methods for treating or preventing Condition in a patient, comprising administering to the patient one or more Compounds of Formula (I) and an additional therapeutic agent that is not a Compound of Formula (I), wherein the amounts administered are together effective to treat or prevent the Condition.

The present invention further provides pharmaceutical compositions comprising an effective amount of one or more compounds of Formula (I) or a pharmaceutically acceptable salt, solvate thereof, and a pharmaceutically acceptable carrier. The compositions can be useful for treating or preventing a Condition in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

The term “patient” as used herein, refers to a human or non-human mammal. In one embodiment, a patient is a human. In another embodiment, a patient is a non-human mammal, including, but not limited to, a monkey, dog, baboon, rhesus, mouse, rat, horse, cat or rabbit. In another embodiment, a patient is a companion animal, including but not limited to a dog, cat, rabbit, horse or ferret. In one embodiment, a patient is a dog. In another embodiment, a patient is a cat.

The term “obesity” as used herein, refers to a patient being overweight and having a body mass index (BMI) of 25 or greater. In one embodiment, an obese patient has a BMI of about 25 or greater. In another embodiment, an obese patient has a BMI of between about 25 and about 30. In another embodiment, an obese patient has a BMI of between about 35 and about 40. In still another embodiment, an obese patient has a BMI greater than 40.

The term “obesity-related disorder” as used herein refers to: (i) disorders which result from a patient having a BMI of about 25 or greater; and (ii) eating disorders and other disorders associated with excessive food intake. Non-limiting examples of an obesity-related disorder include edema, shortness of breath, sleep apnea, skin disorders and high blood pressure.

The term “metabolic syndrome” as used herein, refers to a set of risk factors that make a patient more susceptible to cardiovascular disease and/or type 2 diabetes. As defined herein, a patient is considered to have metabolic syndrome if the patient has one or more of the following five risk factors:
1) central/abdominal obesity as measured by a waist circumference of greater than 40 inches in a male and greater than 35 inches in a female;
2) a fasting triglyceride level of greater than or equal to 150 mg/dL;
3) an HDL cholesterol level in a male of less than 40 mg/dL or in a female of less than 50 mg/dL;
4) blood pressure greater than or equal to 130/85 mm Hg; and
5) a fasting glucose level of greater than or equal to 110 mg/dL.

The term “impaired glucose tolerance” as used herein, is defined as a two-hour glucose level of 140 to 199 mg per dL (7.8 to 11.0 mmol) as measured using the 75-g oral glucose tolerance test. A patient is said to be under the condition of impaired glucose tolerance when he/she has an intermediately raised glucose level after 2 hours, wherein the level is less than would qualify for type 2 diabetes mellitus.

The term “impaired fasting glucose” as used herein, is defined as a fasting plasma glucose level of 100 to 125 mg/dL; normal fasting glucose values are below 100 mg per dL.

The term “upper airway” as used herein, refers to the upper respiratory system, i.e., the nose, throat, and associated structures.

The term “effective amount” as used herein, refers to an amount of compound of formula (I) and/or an additional therapeutic agent, or a composition thereof that is effective in producing the desired therapeutic, ameliorative, inhibitory or preventative effect when administered to a patient suffering from a Condition. In the combination therapies of the present invention, an effective amount can refer to each individual agent or to the combination as a whole, wherein the amounts of all agents administered are together effective, but wherein the component agent of the combination may not be present individually in an effective amount.

The term “alkyl,” as used herein, refers to an aliphatic hydrocarbon group which may be straight or branched and which contains from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is unsubstituted. In another embodiment, an alkyl group is linear. In another embodiment, an alkyl group is branched.

The term “alkenyl,” as used herein, refers to an aliphatic hydrocarbon group containing at least one carbon-carbon double bond and which may be straight or branched and contains from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkyl, aryl, cycloalkyl, cyano, —O-alkyl and —S(alkyl). In one embodiment, an alkenyl group is unsubstituted.

The term “alkynyl,” as used herein, refers to an aliphatic hydrocarbon group containing at least one carbon-carbon triple bond and which may be straight or branched and contains from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of alkyl, aryl and cycloalkyl. In one embodiment, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂— and —CH₂CH(CH₃)CH₂—. An alkylene group may be unsubstituted or substituted by one or more substituents which may be the same or different, each substituent being independently selected from the group consisting of halo, alkyl, aryl, cycloalkyl, cyano, O-alkyl and —S(alkyl). In one embodiment, an alkylene group is unsubstituted. In another embodiment, an alkylene group has from 1 to about 6 carbon atoms. In another embodiment, an alkylene group is branched. In still another embodiment, an alkylene group is linear.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH₂CH═CH—, —CH₂CH═CHCH₂—, —CH═CHCH₂CH₂—, —CH₂CHCH═CH—, —CH(CHOCH═CH— and —CH═C(CH₃)CH₂—. In one embodiment, an alkenylene group has from 2 to about 6 carbon atoms. In another embodiment, an alkenylene group is branched. In another embodiment, an alkenylene group is linear.

The with “alkynylene,” as used herein, refers to an alkynyl group, as defined above, wherein one of the alkynyl group's hydrogen atoms has been replaced with a bond. Non-limiting examples of alkynylene groups include —C≡C—, —CH₂C≡C—, —CH₂C≡CCH₂—, —C≡CCH₂CH₂—, —CH₂CHC≡C—, —CH(CH₃)C≡C— and —C≡CCH₂—. In one embodiment, an alkynylene group has from 2 to about 6 carbon atoms. In another embodiment, an alkynylene group is branched. In another embodiment, an alkynylene group is linear.

The term “aryl” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. Non-limiting examples of aryl groups include phenyl and naphthyl. In one embodiment, an aryl group is unsubstituted. In another embodiment, an aryl group is phenyl.

The term “cycloalkyl,” as used herein, refers to a non-aromatic mono- or multicyclic ring system comprising from about 3 to about 10 ring carbon atoms. In one embodiment, a cycloalkyl contains from about 3 to about 7 ring carbon atoms. In another embodiment, a cycloalkyl contains from about 5 to about 7 ring atoms. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted.

The term “cycloalkenyl,” as used herein, refers to a non-aromatic mono- or multicyclic ring system comprising from about 3 to about 10 ring carbon atoms and containing at least one endocyclic double bond. In one embodiment, a cycloalkenyl contains from about 5 to about 10 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. In one embodiment, a cycloalkenyl group is unsubstituted. In another embodiment, a cycloalkenyl group is a 6-membered cycloalkenyl. In another embodiment, a cycloalkenyl group is a 5-membered cycloalkenyl.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. A heteroaryl group can be optionally substituted by one or more “ring system substituents” which may be the same or different, and are as defined herein below. A heteroaryl group is attached via a ring carbon atom, and any nitrogen atom of a heteroaryl can be optionally oxidized to the corresponding N-oxide. The term “heteroaryl” also encompasses a heteroaryl group, as defined above, which has been fused to a benzene ring. Non-limiting examples of heteroaryls include pyridyl, pyrazinyl, furanyl, thienyl, pyrimidinyl, isoxazolyl, isothiazolyl, oxazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. In one embodiment, a heteroaryl group is unsubstituted. In another embodiment, a heteroaryl group is a 6-membered heteroaryl. In another embodiment, a heteroaryl group is a 5-membered heteroaryl.

The term “heterocycloalkyl,” as used herein, refers to a non-aromatic saturated monocyclic or multicyclic ring system comprising 3 to about 10 ring atoms, wherein from 1 to 4 of the ring atoms are independently O, S or N and the remainder of the ring atoms are carbon atoms. In one embodiment, a heterocycloalkyl group has from about 5 to about 10 ring atoms. In another embodiment, a heterocycloalkyl group has 5 or 6 ring atoms. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include piperidyl, pyrrolidinyl, piperazinyl, pyrrolidonyl, morpholinyl, thiomorpholinyl, thiazolidinyl, 1,4-dioxanyl, tetrahydrofuranyl, tetrahydrothiophenyl, lactam, lactone, and the like. A ring carbon atom of a heterocycloalkyl group may be functionalized as a carbonyl group. An illustrative example of such a heterocycloalkyl group is pyrrolidonyl:

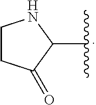

In one embodiment, a heterocycloalkyl group is unsubstituted. In another embodiment, a heterocycloalkyl group is a 6-membered heterocycloalkyl. In another embodiment, a heterocycloalkyl group is a 5-membered heterocycloalkyl.

The term “heterocycloalkenyl,” as used herein, refers to a heterocycloalkyl group, as defined above, wherein the heterocycloalkyl group contains from 3 to 10 ring atoms, and at least one endocyclic carbon-carbon or carbon-nitrogen double bond. In one embodiment, a heterocycloalkenyl group has from 5 to 10 ring atoms. In another embodiment, a heterocycloalkenyl group is monocyclic and has 5 or 6 ring atoms. A heterocycloalkenyl group can be optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include tetrahydroisoquinolyl, tetrahydroquinolyl 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydroaxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like. A ring carbon atom of a heterocycloalkenyl group may be functionalized as a carbonyl group, for example:

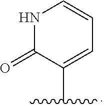

In one embodiment, a heterocycloalkenyl group is unsubstituted. In another embodiment, a heterocycloalkenyl group is a 6-membered heterocycloalkenyl. In another embodiment, a heterocycloalkenyl group is a 5-membered heterocycloalkenyl.

It should also be noted that tautomeric fors such as, for example, the moieties:

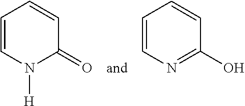

are considered equivalent in certain embodiments of this invention. The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryt, -alkylene-aryl, -alkylene-heteroaryl, -alkenylene-heteroaryl, -alkynylene-heteroaryl, hydroxy, hydroxyalkyl, haloalkyl, —O-alkyl, -alkylene-O-alkyl, —O-aryl, ar-O-alkyl, acyl, aroyl, halo, nitro, cyano, carboxy, —C(O)O-alkyl, —C(O)O-aryl, —C(O)O-alkelene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), Y₁Y₂N—, Y₁Y₂—N-alkyl-, Y₁Y₂—NC(O)— and Y₁Y₂NSO₂—, wherein Y₁ and Y₂ can be the same or different and are independently selected from the group consisting of H, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

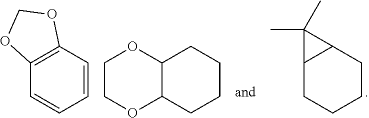

“Halo” means —F, —Cl, —Br or —I. In one embodiment, halo refers to —Cl or —Br.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CH₂Cl and —CCl₃.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃.

The term “alkoxy” as used herein, refers to an —O-alkyl group, wherein an alkyl group is as defined above. Non-limiting examples of —O-alkyl groups include methoxy, ethoxy, n-propoxy, isopropoxy, n-butoxy and t-butoxy. An —O-alkyl group is bonded via its oxygen atom.

The term “substituted” means that one or more hydrogens on the designated atom is replaced with a selection from the indicated group, such that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “purified”, “in purified form” or “in isolated and purified form” for a compound refers to the physical state of the compound after being isolated from a synthetic process (e.g. from a reaction mixture), or natural source or combination thereof. Thus, the term “purified”, “in purified form” or “in isolated and purified form” for a compound refers to the physical state of the compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any variable (e.g., aryl, heterocycle, R², etc.) occurs more than one time in any constituent or in Formula (I), its definition on each occurrence is independent of its definition at every other occurrence, unless otherwise noted.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a Compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a Compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, —O-alkylcarbonyloxymethyl having from 3 to 6 carbon atoms, 1-(—O-alkylcarbonyloxy)ethyl having from 4 to 7 carbon atoms, 1-methyl-1-(—O-alkylcarbonyloxy)ethyl having from 5 to 8 carbon atoms, N—(—O-alkylcarbonyl)aminomethyl having from 3 to 9 carbon atoms, 1-(N—(—O-alkylcarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as 3-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a Compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)—O-alkylcarbonyloxymethyl, N—(C₁-C₆)—O-alkylcarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a OH group of the hemiacetal form of a carbohydrate), and the like.

If a Compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmScirechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The Compounds of Formula (I) can form salts which are also within the scope of this invention. Reference to a Compound of Formula (I) herein is understood to include reference to salts thereof, unless otherwise indicated. The term “salt(s)”, as employed herein, denotes acidic salts formed with inorganic and/or organic acids, as well as basic salts fot wed with inorganic and/or organic bases. In addition, when a Compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a Compound of Formula (I) with an amount of acid or base, such as an equivalent amount, in a medium such as one in which the salt precipitates or in an aqueous medium followed by lyophilization. Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, choline, t-butyl amine, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy group of a —OH compound, in which the non-carbonyl moiety of the carboxylic acid portion of the ester grouping is selected from straight or branched chain alkyl (for example, methyl, ethyl, n-propyl, isopropyl, t-butyl, sec-butyl or n-butyl), —O-alkylalkyl (for example, methoxymethyl), aralkyl (for example, benzyl), —O-alkylene-aryl (for example, phenoxymethyl), aryl (for example, phenyl optionally substituted with, for example, halo, C₁₋₄alkyl, or C₁₋₄—O-alkyl or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Compounds of Formula (I) may be atropisomers (e.g., substituted biaryls) and are considered as part of this invention. Enantiomers can also be separated by use of chiral HPLC column.

It is also possible that the Compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a Compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.).

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of H, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled Compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. In one embodiment, one or more hydrogen atoms of a Compound of Formula (I) are replaced with deuterium atoms. Isotopically labelled Compounds of Formula (I) can generally be prepared using synthetic chemical procedures analogous to those disclosed herein for making the Compounds of Formula (I), by substituting an appropriate isotopically labelled starting material or reagent for a non-isotopically labelled starting material or reagent.

Polymorphic forms of the Compounds of Formula (I), and of the salts, solvates, hydrates, esters and prodrugs of the Compounds of Formula (I), are intended to be included in the present invention.

Unless otherwise stated, the following abbreviations have the stated meanings: boc or BOC is tert-butyoxycarbonyl, BtOH is butanol, tBuOH is tertiary-butanol, dichloromethane is dichloromethane, DIPEA is diisopropylethylamine, DMAP is N,N′-dimethylaminopyridine, DMF is N,N-dimethylformamide, DPPA is diphenylphosphoryl azide, EDC is 1,2-dichloroethane, Et₃N is triethylamine, EtOAc is ethyl acetate, EtOH is ethanol, Et₃SiH is triethylsilyl hydride, HOBt is N-hydroxybenzotriazole, K₂CO₃ is potassium carbonate, KHMDS is potassium hexamethyldisilazide, MeOH is methanol, NaBH(OAc)₃ is sodium triacetoxyborohydride, NBS is N-bromosuceinimide, Ra—Ni is Raney nickel, TFA is trifluoroacetic acid, THF is tetrahydrofuran and TLC is thin layer chromatography.

### The Compounds of Formula (I)

The present invention provides Compounds of Formula (I):

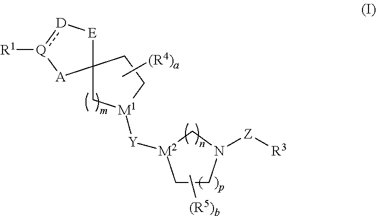

and pharmaceutically acceptable salts, solvates, esters and prodrugs thereof, wherein R¹, R², R³, R⁴, R⁵, A, D, E, M¹, M², Q, Y, Z, a, b, m, n and p are defined above for the Compounds of Formula (I).

In one embodiment, M¹ is —N—.

In another embodiment, M¹ is —CH—.

In one embodiment, M² is —N—.

In another embodiment, M² is —CF— or —CH—.

In another embodiment, M² is —CH—.

In yet another embodiment, M² is —C(F)—.

In one embodiment, Y is —C(O)—.

In another embodiment, Y is alkylene.

In another embodiment, Y is —CH₂—.

In still another embodiment, Y is -alkylene-C(O)—.

In another embodiment, Y is —C(O)-alkylene-.

In another embodiment, Y is —O—.

In still another embodiment, Y is —S(O)₂,

In another embodiment, Y is —C(S)—.

In one embodiment, Z is a bond

In another embodiment, Z is alkylene.

In another embodiment, Z is —CH₂—.

In still another embodiment, Z is —CH(CH₃)—.

In one embodiment, a is 0.

In another embodiment, b is 0.

In one embodiment, m is 1.

In another embodiment, m is 2.

In one embodiment, n is 1.

In another embodiment, n is 2.

In one embodiment, p is 1.

In another embodiment, p is 2.

In one embodiment, m and n are each 2 and p is 1.

In another embodiment, M¹ is —N—; Z is alkylene; m and n are each 2; and p is 1.

In another embodiment, M¹ is —N—; M² is —CH—; Z is alkylene; m and n are each 2; and p is 1.

In still another embodiment, M¹ is —N—; M² is —CH—; Y is —C(O)—; Z is alkylene; m and n are each 2; and p is 1.

In another embodiment, M¹ is —N—; M² is —CH—; Y is —C(O)—; Z is CH₂—; m and n are each 2; and p is 1.

In one embodiment, R³ is aryl or heteroaryl.

In another embodiment, R³ is heteroaryl.

In another embodiment, R³ is 6-membered heteroaryl.

In still another embodiment, R³ is 5-membered heteroaryl.

In another embodiment, R³ is heteroaryl, having at least one ring nitrogen atom.

In one embodiment, R¹ is aryl or heteroaryl, each of which can be optionally substituted as set forth for the Compounds of Formula (I).

In another embodiment, R¹ is a phenyl or pyridyl, each of which can be optionally substituted with one or more groups, each independently selected from halogen, alkyl and —CN.

In one embodiment, R² is aryl, alkyl or hydrogen.

In another embodiment, R¹ and R² join, and together with Q and D, combine to form an aryl or heteroaryl group, each of which can be optionally substituted as set forth above for the Compounds of Formula (I).

In another embodiment, R¹ and R² join, and together with Q and D, combine to form an aryl group or a 5- or 6-membered heteroaryl group, each of which can be optionally substituted as set forth above for the Compounds of Formula (I).

In still another embodiment, R¹ and R² join, and together with Q and D, combine to form an aryl group or a 5- or 6-membered heteroaryl group, each of which can be optionally substituted with one or more substituents, each independently selected from halogen, alkyl and —CN.

In another embodiment, R³ is H or R²-aryl, wherein R²² is H, or —C(O)NH₂.

In a further embodiment R³ is H.

In one embodiment, A is a bond, —CHR⁶—, —O— or —NR⁸—, wherein: R⁶ is hydrogen, alkyl or aryl, and R⁸ is hydrogen, alkyl, haloalkyl, aryl or heteroaryl.

In another embodiment, A is a bond, —O— or —NR⁸—, wherein R⁸ is hydrogen, alkyl or aryl.

In another embodiment, A is a bond or —O—.

In one embodiment, E is —CH₂CH₂—, —CHR⁶CH₂—, —C(O)CH₂—, —C(N—OR⁹)CH₂— or —C(O)—, wherein R⁶ is halogen, alkyl, aryl, heteroaryl, —N(R¹³)₂— or —NHC(O)R¹⁴, and R⁹ is hydrogen, alkyl or haloalkyl.

In another embodiment, E is —CHR⁶CH₂—, —C(O)CH₂— or —C(N—OR⁹)CH₂—, wherein R⁶ is halogen, alkyl, aryl, heteroaryl, —N(R¹³)₂— or —NHC(O)R¹⁴, and R⁹ is hydrogen, alkyl or haloalkyl.

In another embodiment, E is —CHR⁶CH₂— and —C(N—OR⁹)CH₂—, wherein: R⁶ is hydrogen, halogen or alkyl, and R⁹ is hydrogen, alkyl or haloalkyl.

In one embodiment, Z is C₁-C₃ alkylene, —CH(R²⁰)—(R²³—C₁-C₅ alkylene), —CH(R²⁰)—C(R²⁰)═C(R²⁰)—, —(CH₂)₂—O— or C₁-C₃ alkylene interrupted by a cycloalkylene group.

In another embodiment, Z is C₁-C₃ alkylene, —CH₂— (haloalkyl)-, alkenylene, —(CH₂)₂—O— or C₁-C₃ alkylene interrupted by a cycloalkylene group.

In another embodiment, Z is —CH₂—, —(CH₂)₃—, —CH₂—CH═CH—, —(CH₂)₂—CH(F)—, —CH₂—CH(F)—CH₂—, —(CH₂)₂—O— or

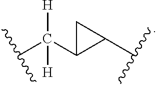

In still another embodiment, Z is —CH₂—.

In yet another embodiment, Z is —CH₂CH═CH—.

In one embodiment, M¹ is —N—, m is 2, a is 0, Y is —C(O)—, M² is —CH—, n is 2, p is 1, R⁵ is halo and h is 0 or 1.

In another embodiment, M¹ is —N—, m is 2, a is 0, Y is —C(O)—, M² is —CH—, n is 2, p is 1, R⁵ is halo, b is 0 or 1, and Z is —CH₂—.

In one embodiment, the spiro ring containing A, E, Q and D is an optionally substituted pyranyl, oxazolidinyl, pyrrolidinyl or cyclopentyl ring, any of which can be optionally fused to an aryl or heteroaryl ring.

In a another embodiment, the spiro ring containing A, E, Q and D is a tetrahydropyranyl ring which is optionally substituted on a ring carbon atom by up to 4 groups, each independently selected from halo, ═N—O—CH₃, ═N—OH, —NHCOCF₃, —NH—CO—N(CH₃)₂ and —NHCOCH₃, and wherein the pyranyl ring can be optionally fused to an optionally substituted phenyl, pyridyl or thienyl ring.

In another embodiment, the spiro ring containing A, E, Q and D is an optionally substituted oxazolidinyl ring.

In another embodiment, the spiro ring containing A, E, Q and D is an optionally substituted pyrrolidin-2-one ring, which is optionally fused to a phenyl ring.

In another embodiment, the spiro ring containing A, E, Q and D is a cyclopentyl ring having one ring carbon atom substituted with a ═N—O—CH₃ group, and wherein the cyclopentyl ring can be optionally fused to a phenyl ring.

In one embodiment, R³ is heterocycloalkyl.

In another embodiment, R³ is heteroaryl.

In one embodiment, R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂.

In another embodiment, R³ is:

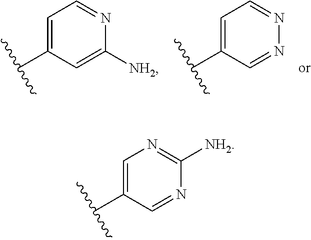

In another embodiment, R³ is:

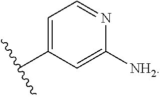

In one embodiment, Y is —C(O)— and Z is alkylene.

In another embodiment, Y is —C(O)— and Z is —CH₂—.

In one embodiment, Y is —C(O)—, Z is —CH₂— and R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂.

In one embodiment, ring:

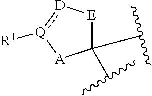

is:

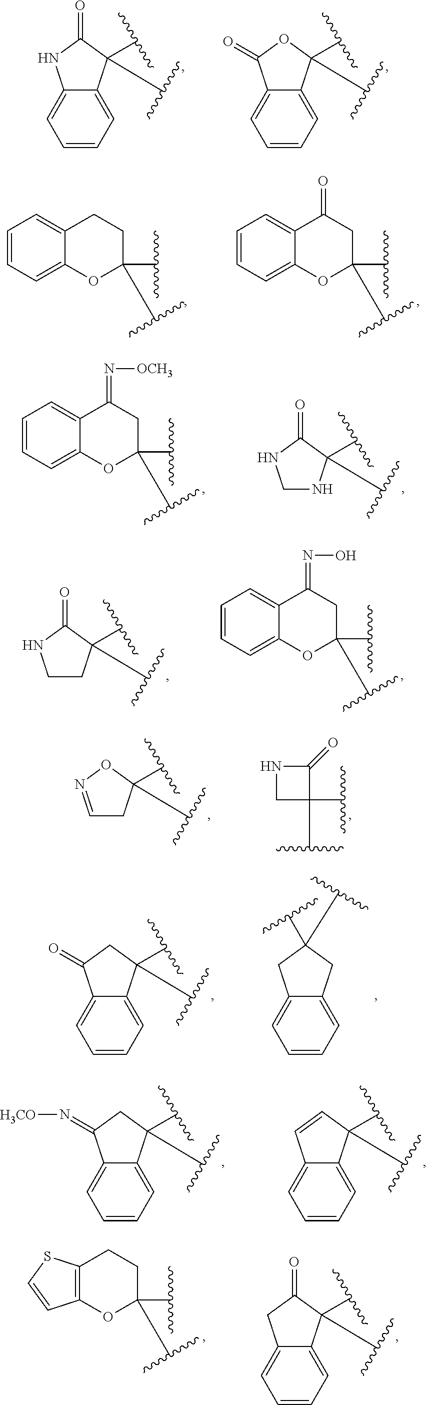

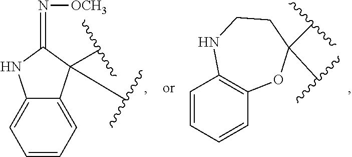

each of which can be optionally substituted with up to 3 substituents, each independently selected from alkyl, phenyl, —NHC(O)—R²¹, halo, benzyl, —NHS(O)₂-alkyl or —NHC(O)N(R²²)₂, wherein R²¹ is alkyl, heteroaryl or haloalkyl, and R²² is H or alkyl.

In another embodiment, Y is —C(O)—; Z is —CH₂—; R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂; and ring:

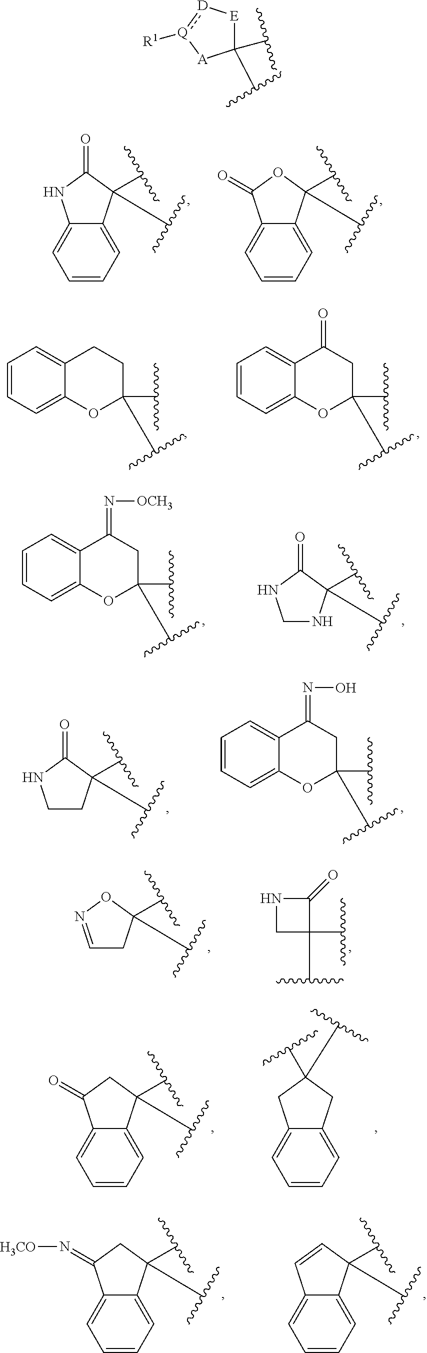

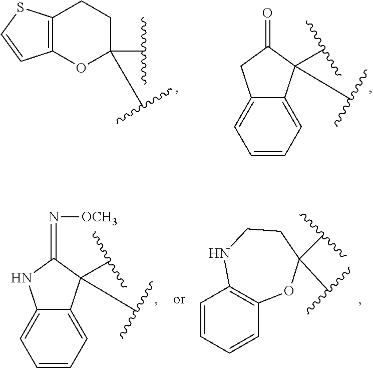

is:
each of which can be optionally substituted with up to 3 substituents, each independently selected from alkyl, phenyl, —NHC(O)—R²¹, halo, benzyl, —NHS(O)₂-alkyl or —NHC(O)N(R²²)₂, wherein R²¹ is alkyl, heteroaryl or haloalkyl, and R²² is H or alkyl.

In one embodiment, for the Compounds of Formula (I), R¹, R², R³, R⁴, R⁵, A, D, E, M¹, M², Q, Y, Z, a, b, m, n and p are selected independently from each other.

In another embodiment, a Compound of Formula (I) is in purified form.

In one embodiment, the Compounds of Formula (I) have the formula (Ia):

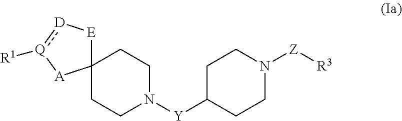

and pharmaceutically acceptable salts, solvates, esters and prodrugs thereof, wherein:

A is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)—, —C(═NH)N(R⁸)— or —N(R⁸)— or —C(R²¹)═N—;

D is —C(R²)— or —N— when the optional and additional bond is present, and D is —C(R²)₂— or —N(R²)— when the optional and additional bond is absent, such that when D is N and optional and additional bond is present, E is either —OC(R²¹)— or —O—;

E is a bond, —CH(R²¹)CH(R⁶)—, —CH(R²¹)C(O)—, —CH(R²¹)C(═N—OR⁹)—, —CH(R²¹)—, —C(O)CH(R⁶)—, —C(O)—, —C(═N—OR⁹)CH(R⁶)—, —C(═N—OR⁹)—, —C(═NH)N(R⁸)—-OCH(R²¹)— or —O— when D is —N—; and E is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —N(R⁸)CH(R⁶)CH(R²¹)—, —N(R⁸)C(O)CH(R²¹)—, —N(R⁸)C(═N—OR⁹)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)— or —N(R⁸)— when D is other than —N—;

Q is —C— when the optional and additional bond is present, and Q is —CH— when the optional and additional bond is absent;

Y is alkylene or —C(O)—;

Z is a bond, alkylene or alkenylene;

R¹ and R² are each independently H, aryl, -alkylene-aryl, heteroaryl, -alkylene-heteroaryl, alkyl, cycloalkyl or heterocycloalkyl, any of which can be optionally substituted with up to 4 substituents, which can be the same or different, and are selected from halo, —OH, alkyl, haloalkyl, —O-alkyl, —O-haloalkyl, —NO₂, —CO₂R^{12, —N(R}¹²)₂, —CON(R¹²)₂, —NHC(O)R¹², —NHSO₂R¹², —SO₂N(R¹²)₂ and —CN, or R¹ and R², together with Q and D, combine to form an aryl, hetroaryl, cycloalkyl or heterocycloalkyl ring, any of which can be optionally substituted with up to 4 substituents, which can be the same or different, and are selected from halo, —OH, alkyl, haloalkyl, —O-alkyl, —O-haloalkyl, —NO₂, —CO₂R¹², —N(R¹²)₂, —CON(R¹²)₂, —NHC(O)R¹², —NHSO₂R¹², —SO₂N(R¹²)₂ and —CN, such that R² is absent when D is nitrogen and the optional and additional bond is present;

R³ is heterocycloalkyl or heteroaryl, each of which can be optionally substituted with up to 3 groups, each independently selected from alkyl, —N(R²⁰)₂ or —OR²⁰; and

each occurrence of R²⁰ is independently H or alkyl.

The following embodiments refer to formula (Ia):

In one embodiment, Y is —C(O)—.

In one embodiment, Z is alkylene.

In another embodiment, Z is alkenylene.

In another embodiment, Z is —CH₂—.

In still another embodiment, Z is —CH₂CH═CH—.

In one embodiment, Y is —C(O)— and Z is alkylene.

In another embodiment, Y is —C(O)— and Z is CH₂—.

In one embodiment, R³ is heterocycloalkyl.

In another embodiment, R³ is heteroaryl.

In one embodiment, R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂.

In another embodiment, R³ is:

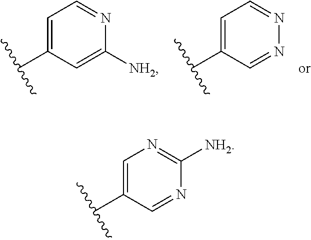

In another embodiment, R³ is:

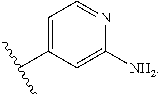

In one embodiment, Y is —C(O)—, Z is —CH₂— and R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂.

In one embodiment, ring:

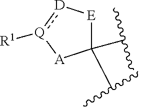

is:

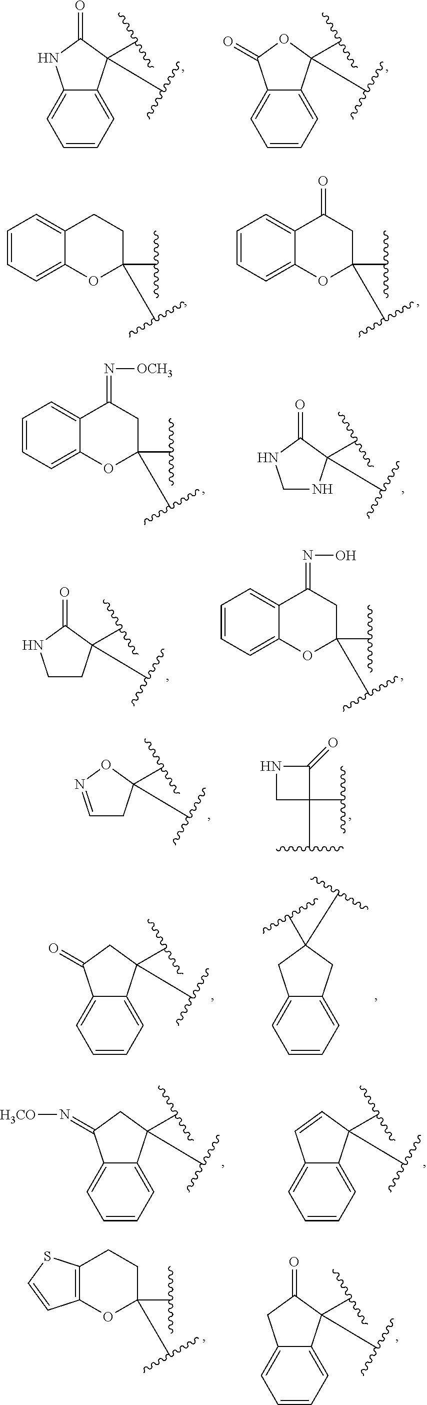

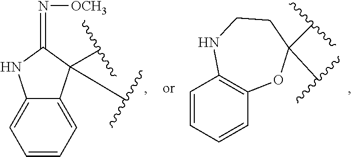

each of which can be optionally substituted with up to 3 substituents, each independently selected from alkyl, phenyl, —NHC(O)—R²¹, halo, benzyl, —NHS(O)₂-alkyl or —NHC(O)N(R²²)₂, wherein R²¹ is alkyl, heteroaryl or haloalkyl, and R²² is H or alkyl.

In another embodiment, Y is —C(O)—; Z is —CH₂—; R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂; and ring:

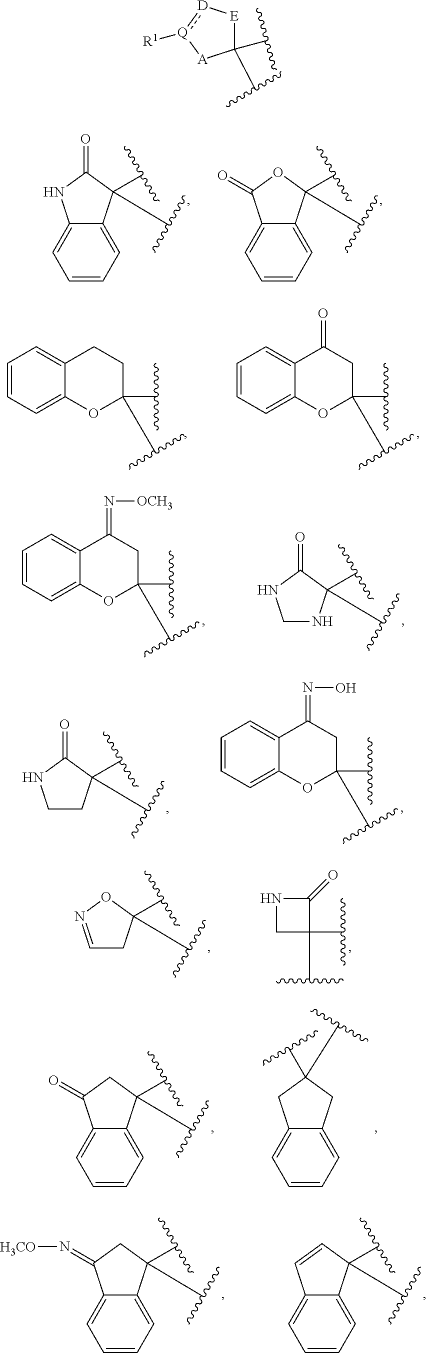

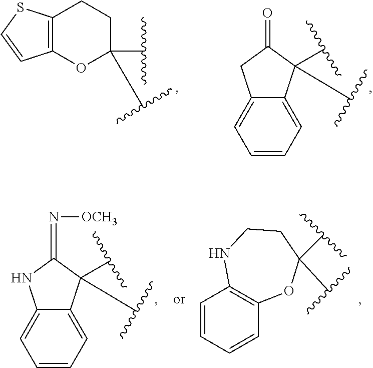

is:
each of which can be optionally substituted with up to 3 substituents, each independently selected from alkyl, phenyl, —NHC(O)—R²¹, halo, benzyl, —NHS(O)₂-alkyl or —NHC(O)N(R²²)₂, wherein R² is alkyl, heteroaryl or haloalkyl, and R²² is H or alkyl.

In one embodiment, for the Compounds of Formula (Ia), R¹, R², R³, A, D, E, Q, Y and Z are selected independently from each other.

In another embodiment, a Compound of Formula (Ia) is in purified form.

Non-limiting examples of the Compounds of Formula (I) include compounds 1-102 as set forth below:

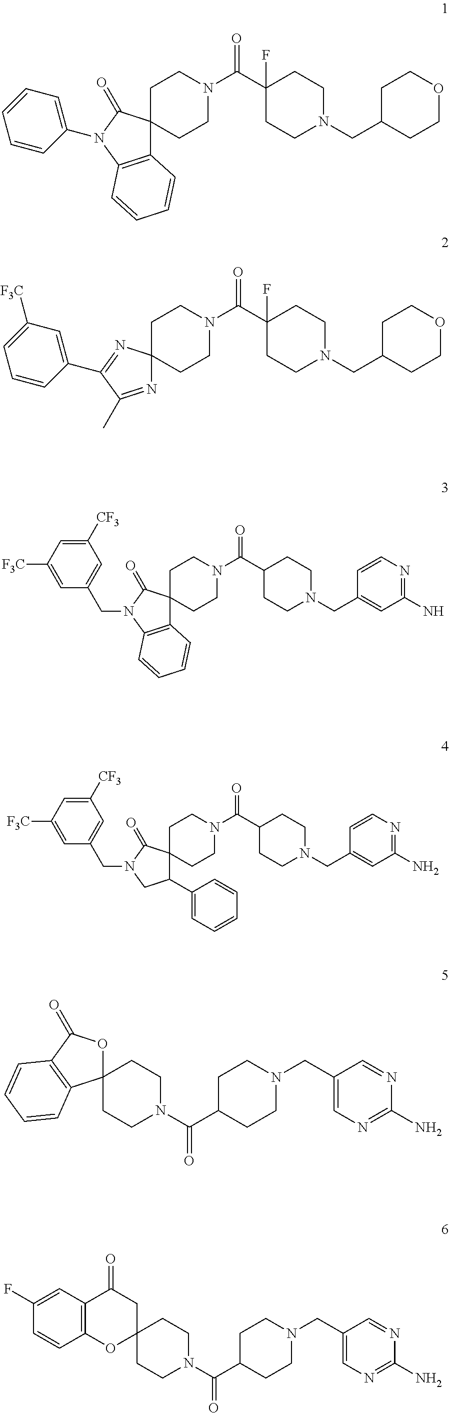

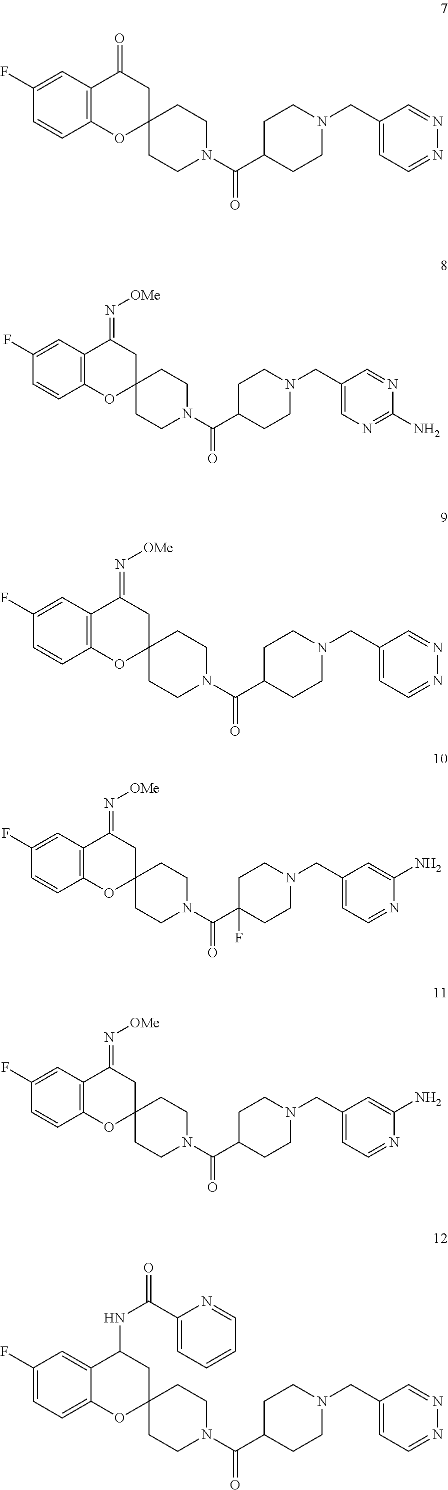

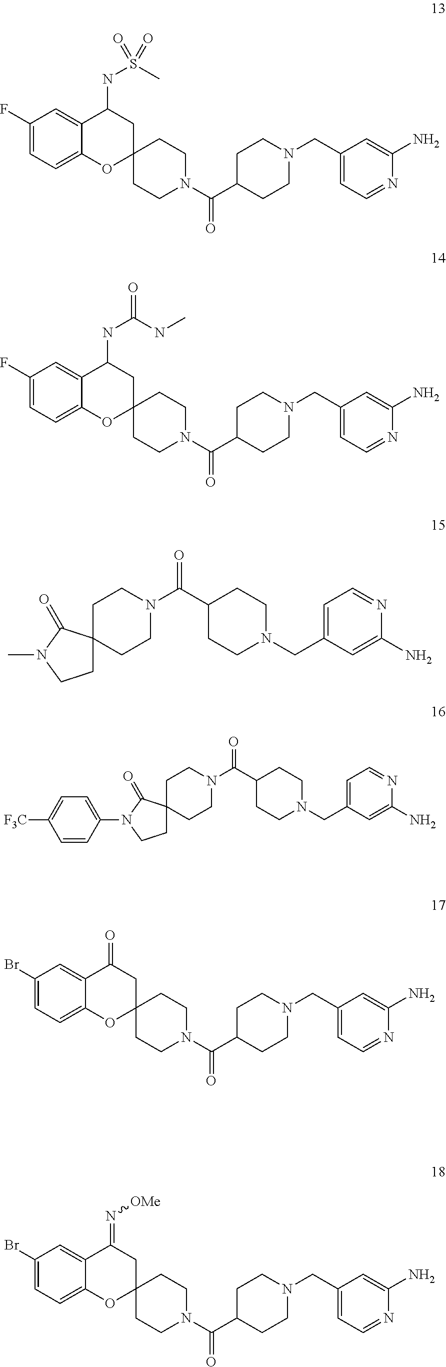

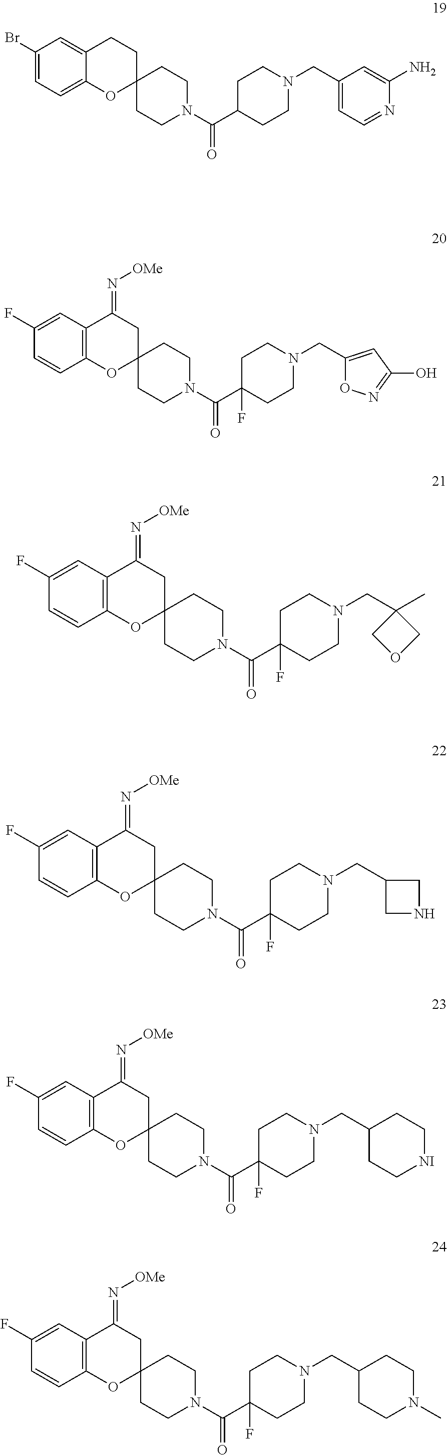

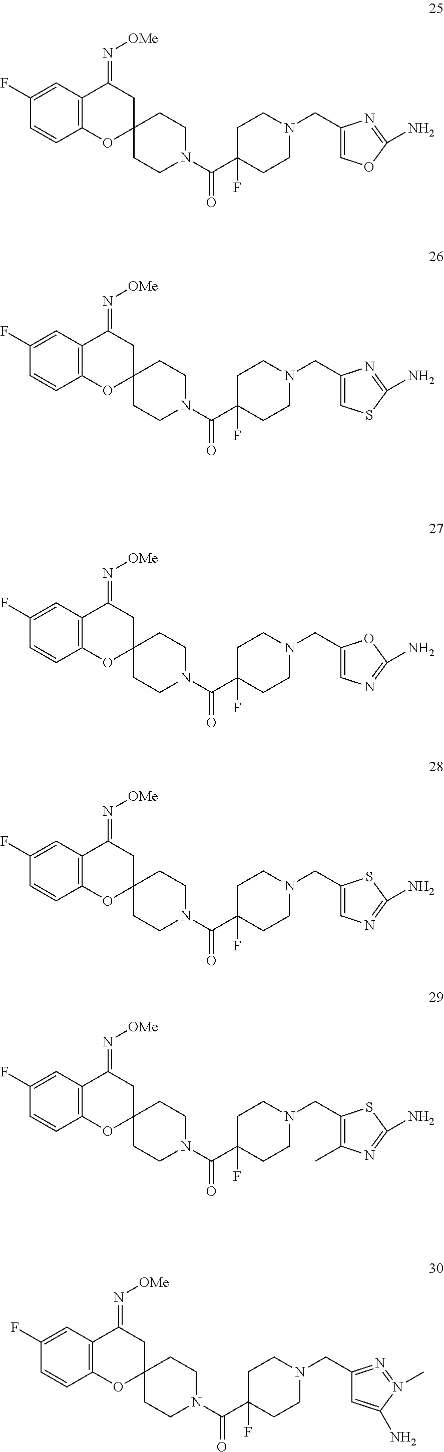

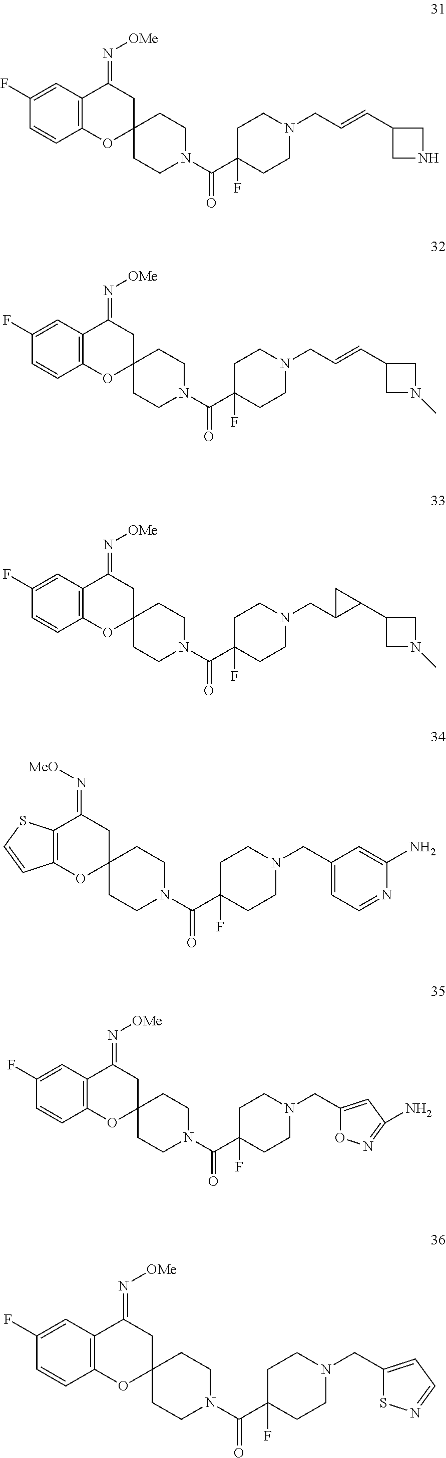

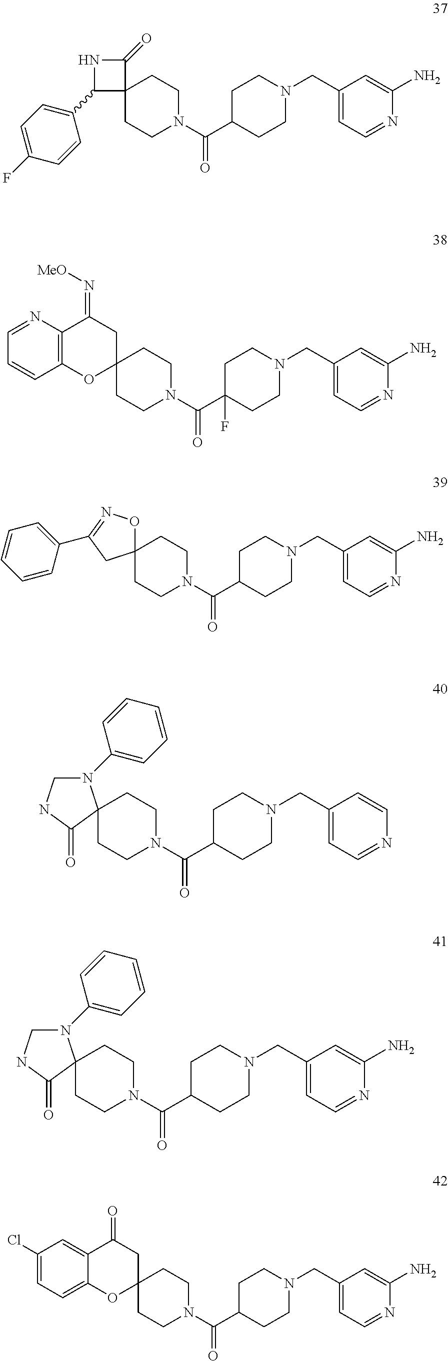

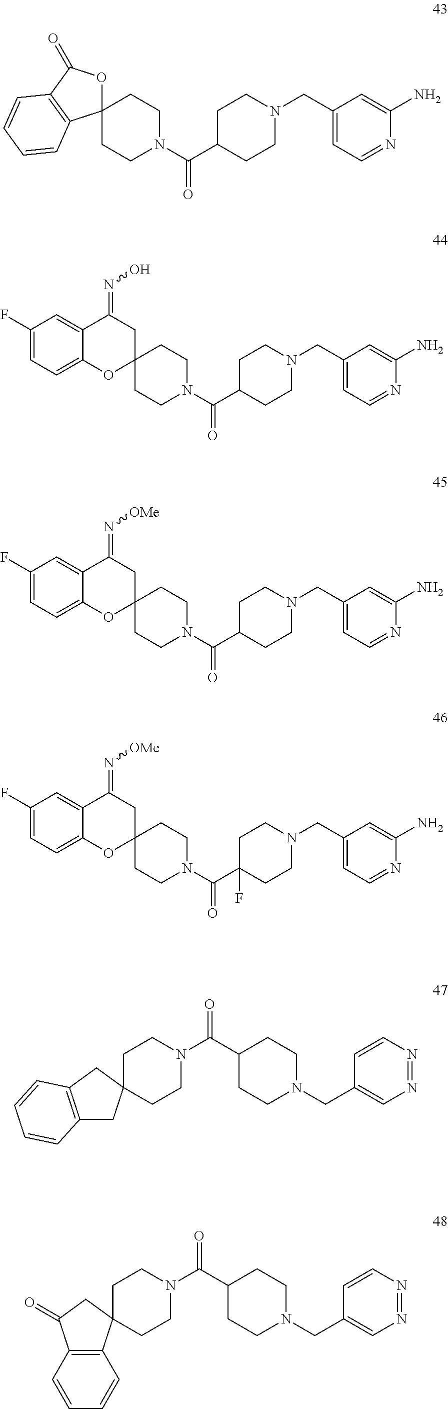

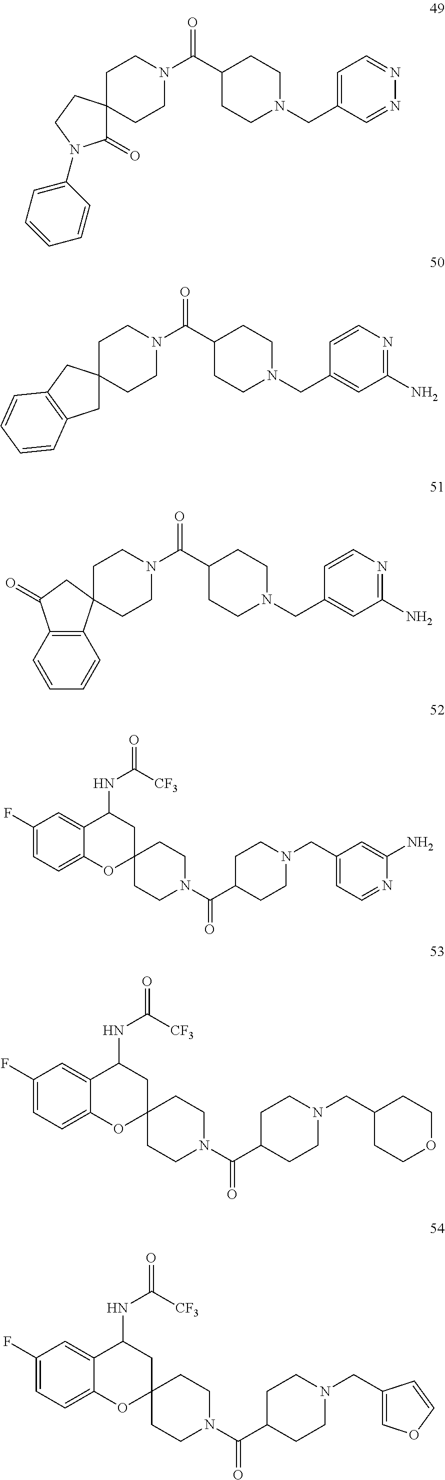

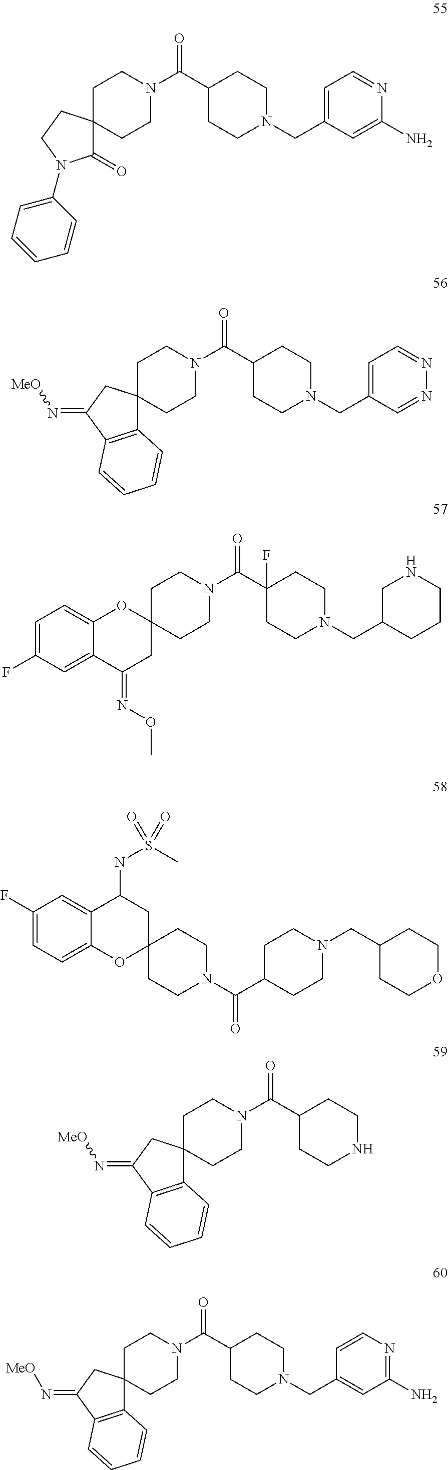

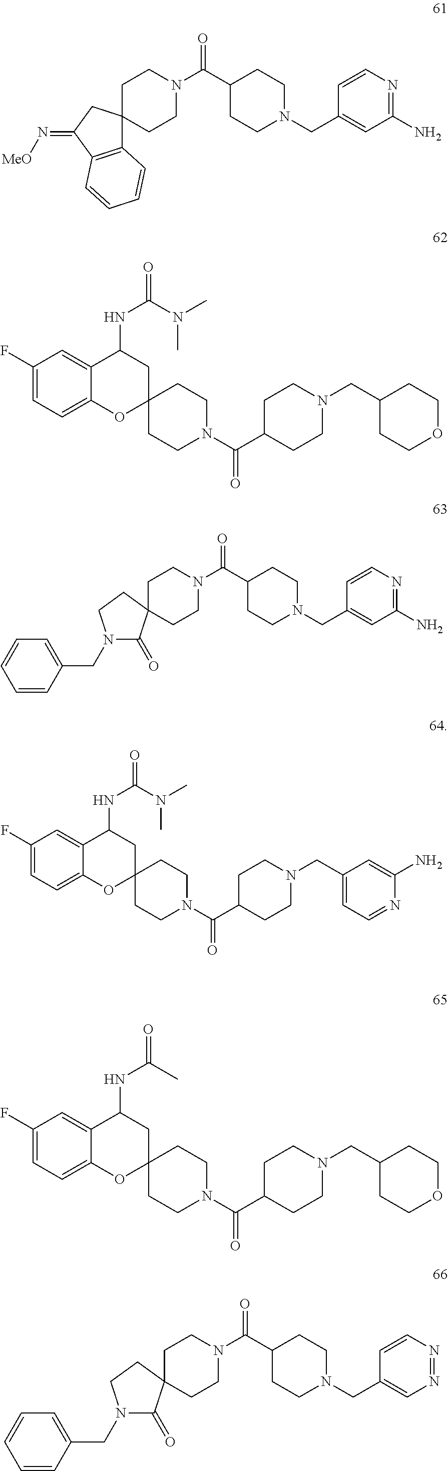

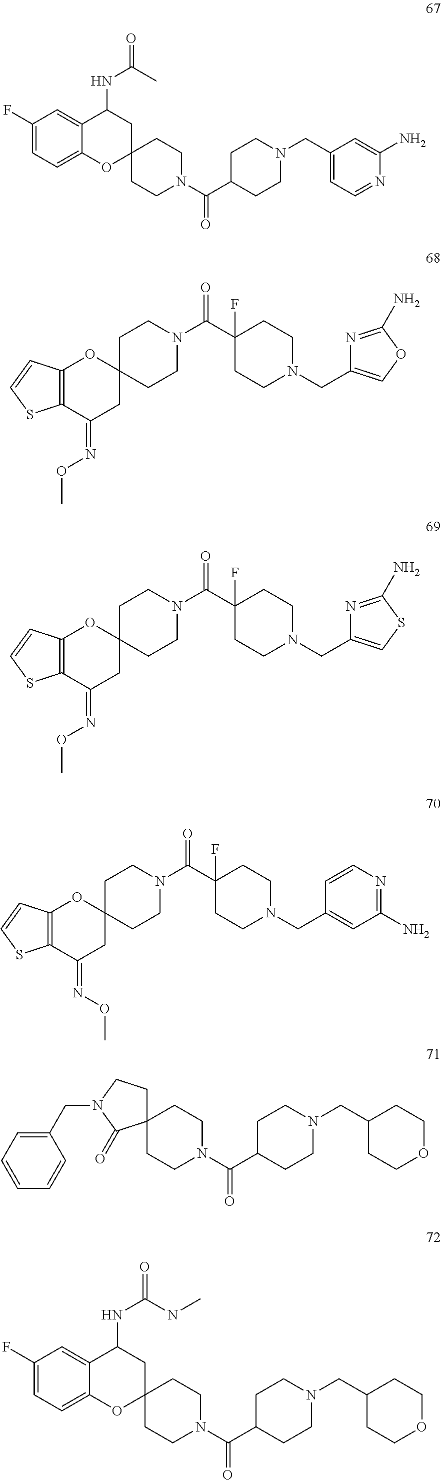

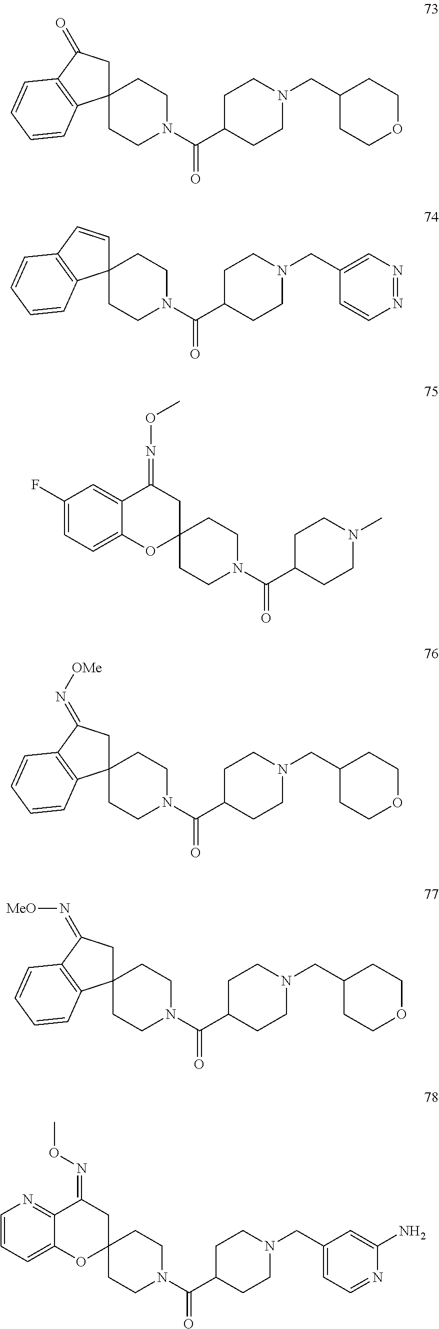

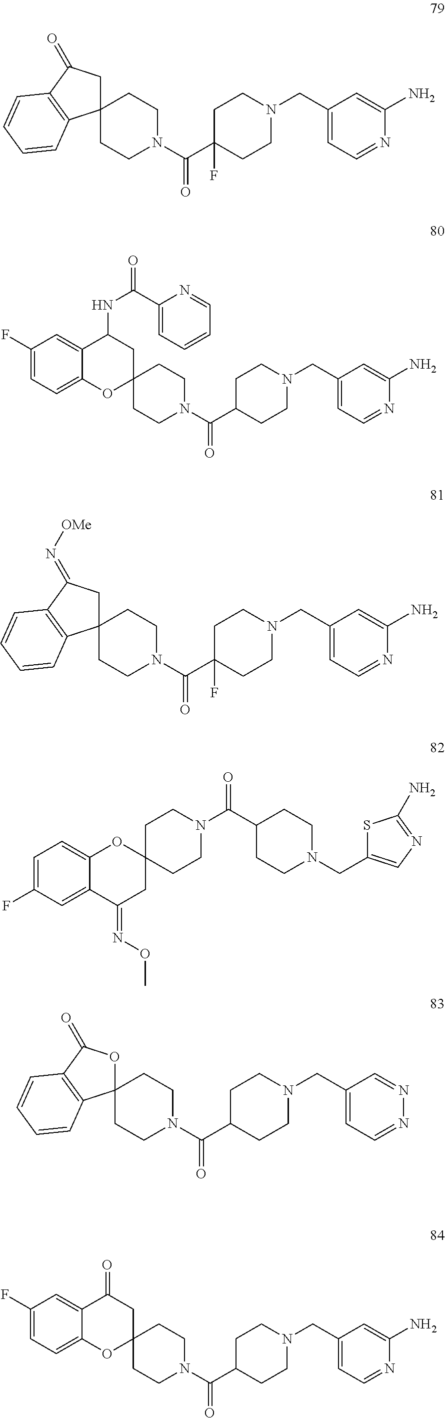

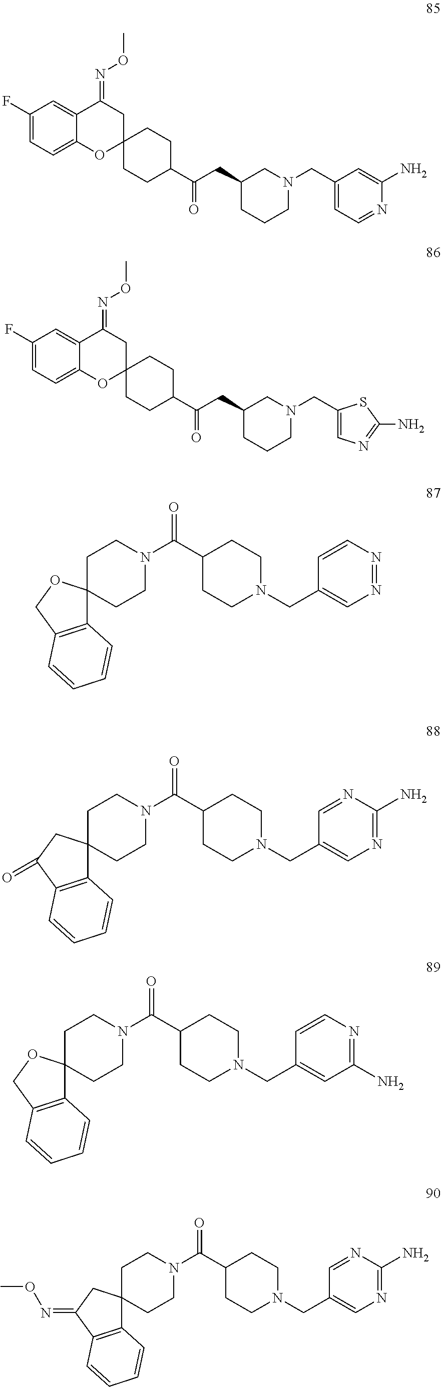

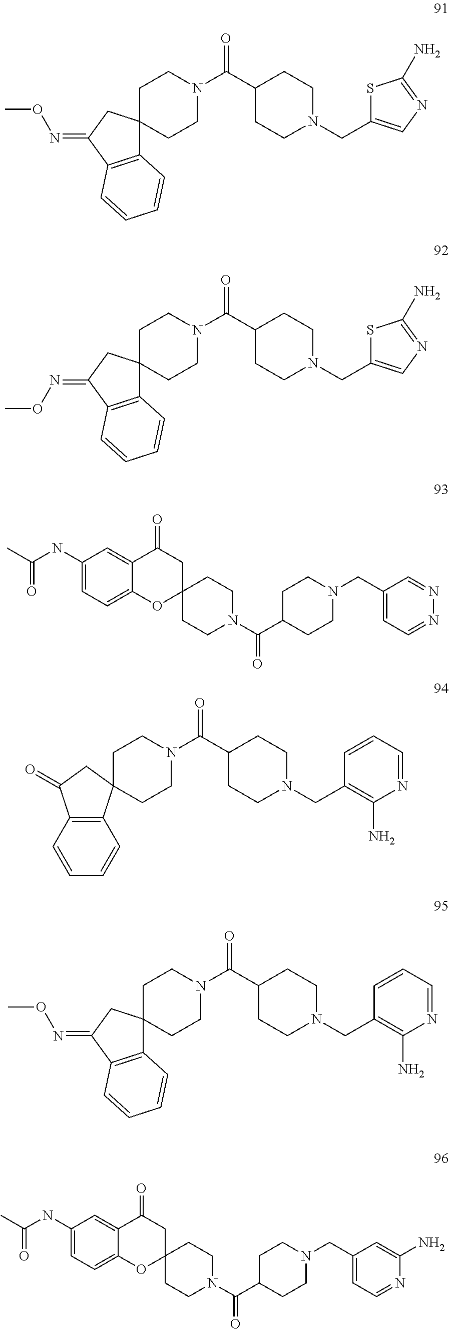

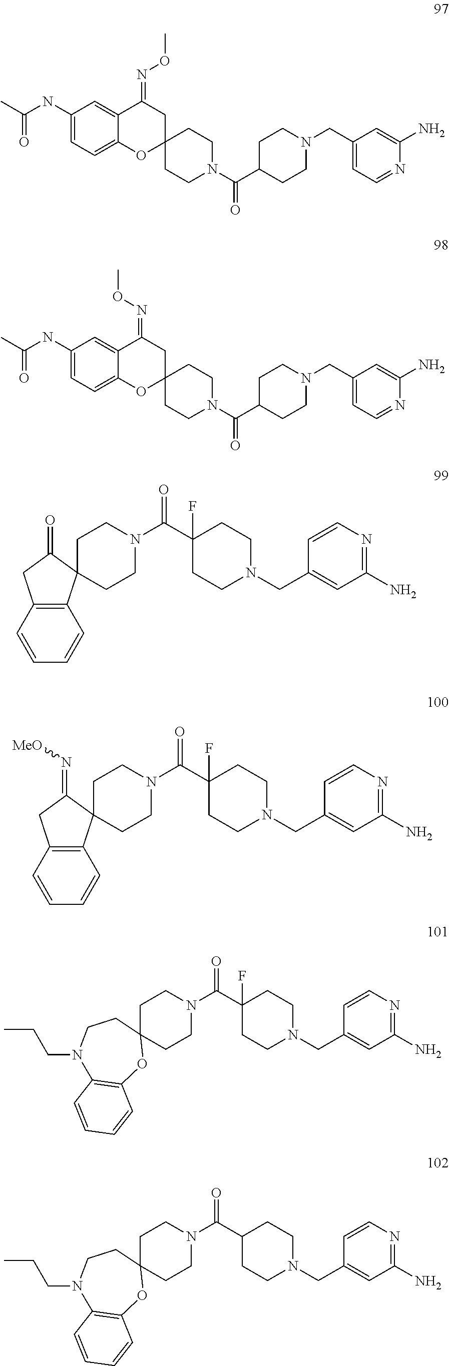

and pharmaceutically acceptable salts, solvates, prodrugs and esters thereof.

### Methods For Making the Compounds of Formula (I)

Methods useful for making the Compounds of Formula (I) are set forth in the Examples below and generalized in Schemes 1-7. Alternative synthetic pathways and analogous structures will be apparent to those skilled in the art of organic synthesis.

As illustrated immediately below, the Compounds of Formula (I) are comprised of a left-hand spirocyclic moiety, designated below as AB, joined, via a linker, Y, to a right-hand heterocyclic moiety, designated as C, which is further derivatized with the group —Z—R³, designated as D.

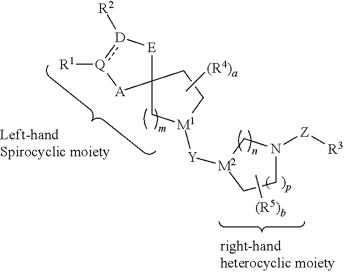

Scheme 1 depicts a method useful for making compounds of formula ABC, which are useful intermediates for making the Compounds of Formula (I), wherein M¹ is —N—, M² is —CH— and Y is —C(O)—.

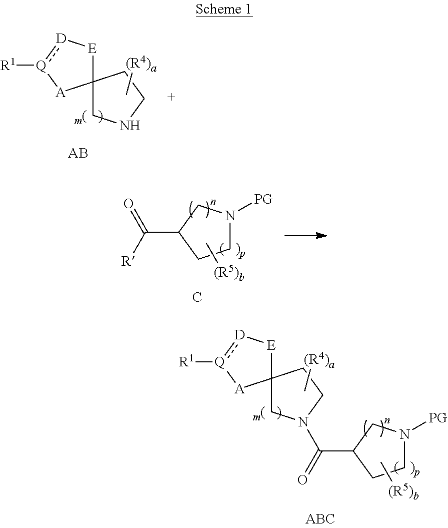

wherein R′ is —O⁺Li⁻ or —Cl, PG is a suitable amine protecting group, and A, D, E, Q, R¹, R⁴, R⁵, a, b, m, n and p are defined above for the Compounds of Formula (I).

The ring nitrogen atom of a spirocyclic compound of formula AB can be reacted with a carboxylic acid salt or acid chloride compound of formula C to provide the intermediates of formula ABC.

Scheme 2 depicts a method useful for making the compounds of formula ABCD, which correspond to the Compounds of Formula (I), wherein M¹ is —N—, M² is —CH— and Y is —C(O)—.

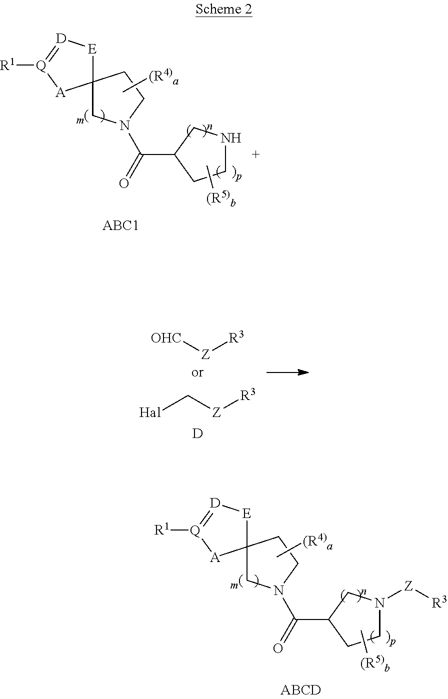

wherein Hal is —Cl, —Br or —I, and A, D, E, Q, Z, R¹, R³, R⁴, R⁵, a, b, m, n and p are defined above for the Compounds of Formula (I).

The nitrogen atom of a spirocyclic compound of formula ABC1, obtained via deprotection of intermediate ABC, can be reacted with an aldehyde or an organohalide of formula D to provide the compounds of formula ABCD, which correspond to the Compounds of Formula (I), wherein M¹ is —N—, M² is —CH— and Y is —C(O)—.

Scheme 3 depicts an alternate method useful for making compounds of formula ABCD, which correspond to the Compounds of Formula (I), wherein M¹ is —N—, M² is —CH— and Y is —C(O)—.

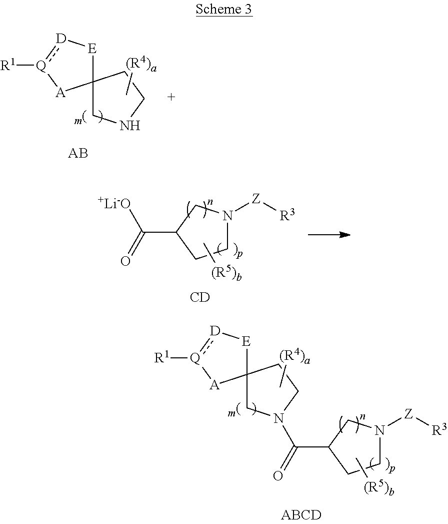

wherein. A, D, E, Q, Z, R¹, R³, R⁴, R⁵, a, b, m, n and p are defined above for the Compounds of Formula (I).

The nitrogen atom of a spirocyclic compound of formula AB can be reacted with a carboxylic acid salt or acid chloride compound of formula CD to provide the compounds of formula ABCD, which correspond to the Compounds of Formula (I), wherein M¹ is —N—, M² is —CH— and Y is —C(O)—.

Various methods useful for making C and D fragments, intermediates of formula CD, and for coupling of C and then D fragments onto an AB group have been previously described in detail in, for example, U.S. Pat. No. 6,720,378 and U.S. Patent Publication Nos. 2007/0015807. C ring fragments may be derived from 4-substituted piperidines (e.g., isonipecotic acid, 4-hydroxypiperidine) or via their ring homologs through appropriate functionalization (e.g., electrophilic fluorination, hydroxylation, alkylation, etc.). In one method, D-type electrophiles are a one-carbon aldehyde or alkyl halide attached to an R³ group (Z is a bond). Longer-chain D-type electrophiles can be synthesized through chain extension of one-carbon starting D aldehydes (previously described or commercially available) by various methods known to those skilled in the art, including, but are not limited to, the reactions of starting aldehydes with alkylmetal reagents, carbon-phosphorus reagents (Wittig reactions and Horner-Emmons reactions), and reactions with other carbon nucleophiles, followed by appropriate functional elaboration, to obtain compounds where Z is an appropriately substituted alkyl or alkenyl group. Alternatively, in the particular case when R³ is an aryl or heteroaryl, the corresponding D fragment with the elongated Z moiety is prepared by coupling an aryl halide with an appropriate alkyl or alkenyl metal (e.g., organolithium or Grignard reagent) reagent, optionally in the presence of an appropriate transition metal catalyst (e.g, Cu, Ni).

Scheme 4 illustrates various methods useful for linking together an A fragment and a B fragment or B fragment precursor to form a spirocyclic AB moiety.

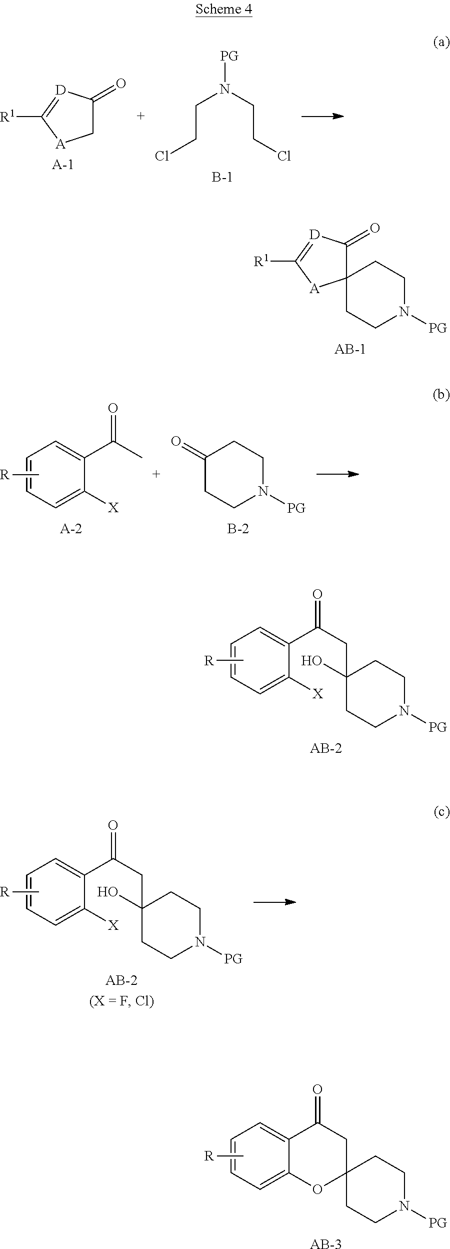

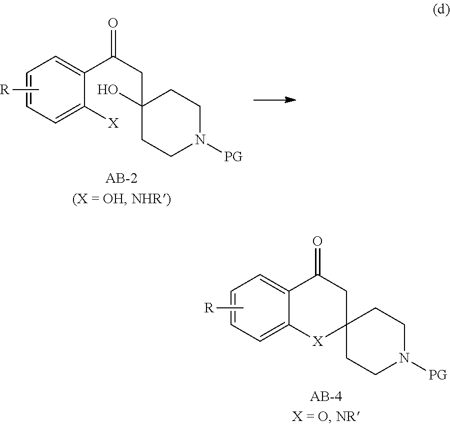

In one approach, shown in Scheme 4(a), the spiro connection is established by building the B ring (most commonly a piperidine ring) from open chain precursor 13-1. This approach is most conveniently exercised, when a carbonyl group is present in the A ring next to the future spiro carbon atom. Double alkylation of the corresponding enolate will then install the spirocyclic linkage. The original carbonyl group can later be elaborated into a different functionality, depending on the final target. Alternatively, Scheme 4(b) shows how the spirocyclic linkage can be established in a stepwise manner starting with piperidine B ring (B-2) with appropriate electrophilic functionality, most conveniently, a carbonyl group, at the future spirocyclic carbon site. This approach will employ a preinstalled nucleophilic functionality or precursor thereof (e.g., methyl ketone precursor of ketone enolate) on the A ring fragment. Addition of the enolate to the B-ring ketone will establish an initial connection between A and B rings, resulting in compound AB-2. Completion of the spiro linkage can be accomplished via an intramolecular reaction between the A and B fragments which are both present in compound AB-2. In Scheme 4(c), the tertiary alcohol is reacted with reaction with a preinstalled electrophilic functionality on the A ring (e.g., halogen) to provide the completed spirocyclic linkage. Alternatively, Scheme 4(d) shows how formation of a carbocation or a carbocation-like intermediate from the intermediate tertiary alcohol AB-2 can be utilized in the reaction with a second nucleophilic functionality, preinstalled on ring A. The transformation described in Scheme 4(d) can be accomplished in a single-step process or can be separated into different steps, if A ring nucleophilic group is initially protected (e.g., as a benzyl ether, benzyl carbamate or other known functionalities, using methods to those skilled in the art of organic synthesis).

Scheme 5 illustrates various methods useful for making spirocyclic AB fragments wherein the spiro fragment is preinstalled into ring B. Ring A is then added onto through alkylation of the carbonyl compound B-5 with an electrophile, the nature of which will depend on the final target.

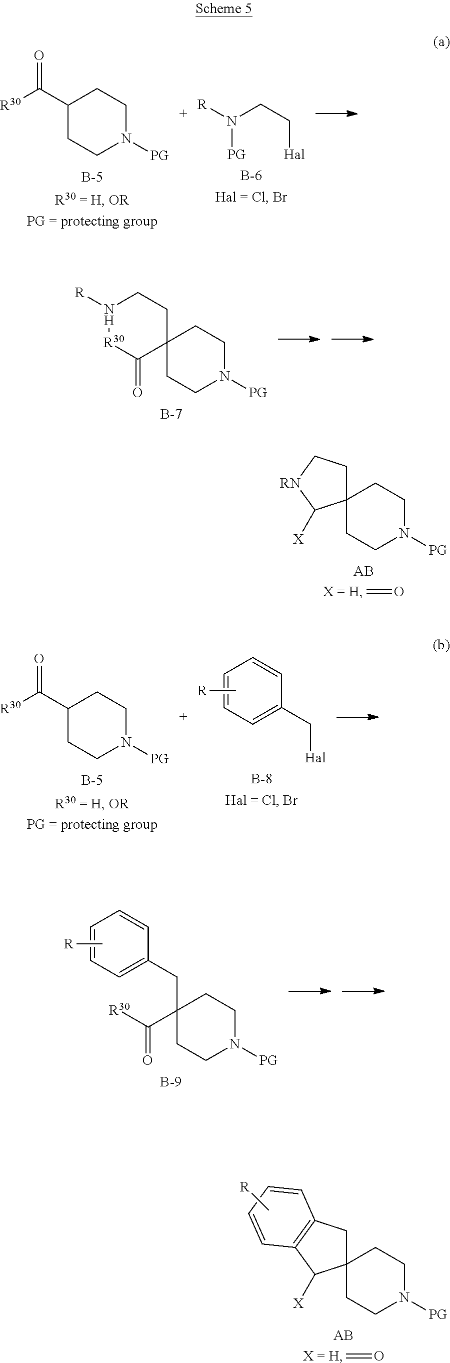

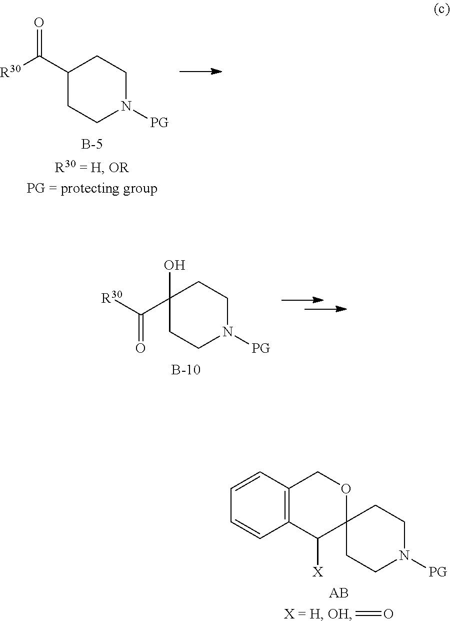

Scheme 5(a) illustrates a method for making a spirocyclic AB group wherein both A ring atoms adjacent to the spiro carbon are each a carbon atom. In addition, this approach can also accommodate a heteroatom next to the spiro carbon, which can be installed through enolate α-hydroxylation or α-amination methods, known to those skilled in the art (Scheme 5(b)). A subsequent cyclization (e.g., via Friedel-Crafts reaction, an amidation process or a reductive amination process) can then be used to form the A ring, optionally fused to another ring (e.g., phenyl). This processes are depicted in Schemes 5(b) and 5(c). The nature of substituent X on the final AB fragment will depend on the starting B ring and the nature of the cyclization reaction used (e.g., reductive vs. nonreductive). X can be further modified post-cyclization, if desired.

Scheme 6 illustrates how cycloaddition methodology can be used to make a spirocyclic AB moiety.

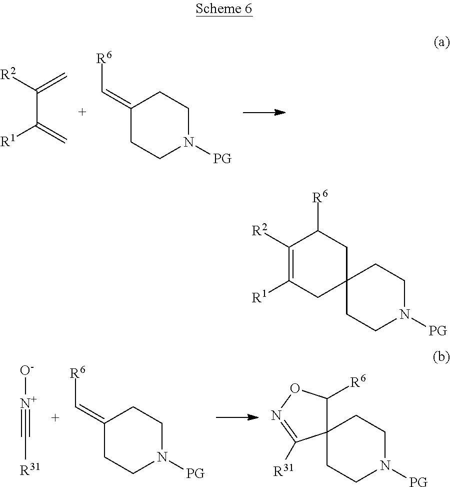

Scheme 6(a) shows how a 4+2 cycloaddition reaction (e.g., Diels-Alder) can be used to construct an AB spirocycle with a 6-membered A ring. Scheme 6(b) illustrates how a cycloaddition process (e.g., Hetero Diels-Alder reaction) can be used with different types of 1,3-dipoles to provide access to AB moieties with heterocyclic A rings.

Scheme 7 illustrates a method useful for making AB fragments wherein the A ring has two heteroatoms that are adjacent to the spiro carbon atom.

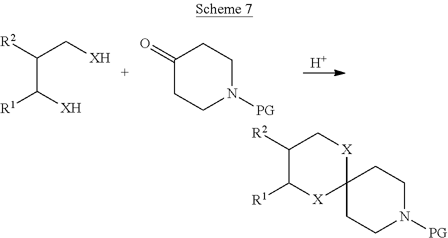

wherein each X is independently O or N, PG is an amine protecting group, and R¹ and R² are defined above for the compounds of formula (I).

As shown in Scheme 7, spirocyclic AB fragments wherein both A ring atoms adjacent to the spiro carbon are heteroatoms, can be assembled through standard ketal aminal formation reactions known to those skilled in the art.

In addition, various approaches to the synthesis of spiro azacycles are also described in International Publication No. WO 94/29309. Similar synthetic routes can be used to access AB portion of the compounds of the present invention. Additionally, a number of spiro azacycles are available commercially and can be employed as reactants in the synthesis of compounds of the present invention.

The starting materials and reagents depicted in Schemes 1-7 are either available from commercial suppliers such as Sigma-Aldrich (St. Louis, Mo.) and Acros Organics Co. (Fair Lawn, N.J.), or can be prepared using methods well-known to those of skill in the art of organic synthesis.

The skilled artisan will recognize that the synthesis of compounds of Formula (I) may require the need for the protection of certain functional groups (i.e., derivatization for the purpose of chemical compatibility with a particular reaction condition). Suitable protecting groups for the various functional groups of the Compounds of Formula (I) and methods for their installation and removal may be found in Greene et al., Protective Groups in Organic Synthesis, Wiley-Interscience, New York, (1999).

### EXAMPLES

### General Methods

The starting materials and reagents used in preparing compounds described are either available from commercial suppliers such as Aldrich Chemical Co. (Wisconsin, USA) and Acros Organics Co. (New Jersey, USA) or were prepared using methods well-known to those skilled in the art of organic synthesis. All commercially purchased solvents and reagents were used as received. LCMS analysis was performed using an Applied Biosystems API-100 mass spectrometer equipped with a Shimadzu SCL-10A LC column: Altech platinum C18, 3 um, 33 mm×7 mm ID; gradient flow: 0 minutes, 10% CH₃CN; 5 minutes, 95% CH₃CN; 7 minutes, 95% CH₃CN; 7.5 minutes, 10% CH₃CN; 9 minutes, stop. Flash column chromatography was performed using Selecto Scientific flash silica gel, 32-63 mesh. Analytical and preparative TLC was performed using Analtech Silica gel GF plates. Chiral HPLC was performed using a Varian PrepStar system equipped with a Chiralpak OD column (Chiral Technologies).

### Example 1

### Preparation of Compound 1

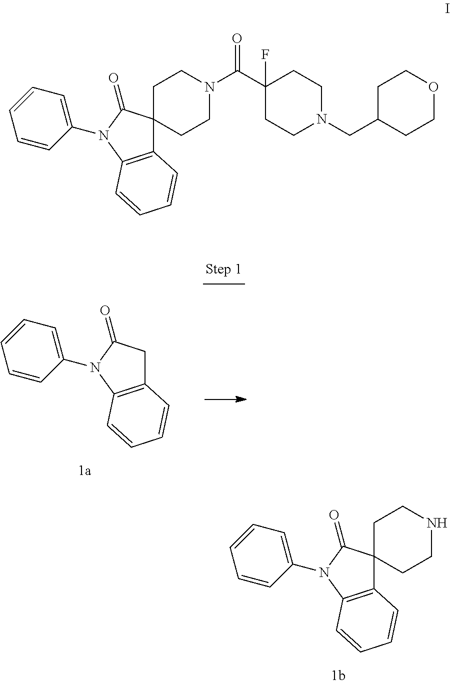

Sodium hydride (50% dispersion in mineral oil, 0.235 g, 4.8 mmol) was added to a stirred solution of 1,3-dihydro-1-phenyl-2H-indol-2-one 1a (2.0 g, 9.6 mmol) in dimethylacetamide (25 mL). To the resulting solution was added bis(2-chloroethyl)amine (1.7 g, 9.6 mmol) in benzene (25 mL) at room temperature. The resulting reaction was heated to about 50° C. and allowed to stir at this temperature for 0.5 hours, then additional sodium hydride (0.235 g, 4.8 mmol) was added and stirring was continued for 2 hours. The reaction mixture was cooled to room temperature, diluted with benzene and treated with water. The organic phase was dried over MgSO₄, filtered and concentrated in vacuo to provide compound 1b.

### Step 2

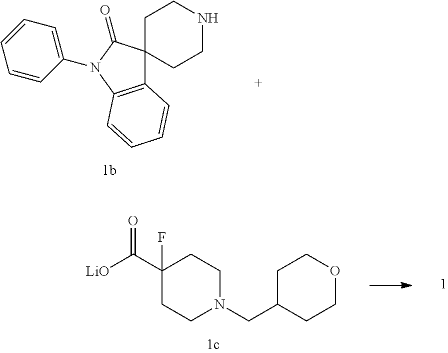

A sealed tube (15 mL) was charged with spiropiperidine 1b (36.3 mg, 0.115 mmol), 1-tetrahydropyran-4-ylmethyl-piperidine-4-lithium-carboxylate 1c (38 mg, 0.150 mmol, 1.3 eq., was prepared as described in U.S. Patent Publication No. 2007/0015807), EDC (33 mg, 0.173 mmol, 1.5 eq), BtOH (23 mg, 0.173 mmol, 1.5 eq), DIPEA (0.1 mL) and dichloromethane (2 mL). The resulting mixture was heated to 65° C. for 15 hours, then cooled to room temperature, diluted with dichloromethane (50 mL) and washed with 1 N aqueous NaOH (30 mL). The layers were separated and the aqueous was extracted with dichloromethane (2×25 mL). The combined organic phase was dried over MgSO₄, filtered and concentrated in vacuo to provide a crude oil that was purified using preparative TLC (SiO₂, dichloromethane:0.4 N NH₃ in MeOH 95:5) to provide compound 1 (24 mg, 41%) as a white solid. MH⁺=506

### Example 2

### Preparation of Compound 2

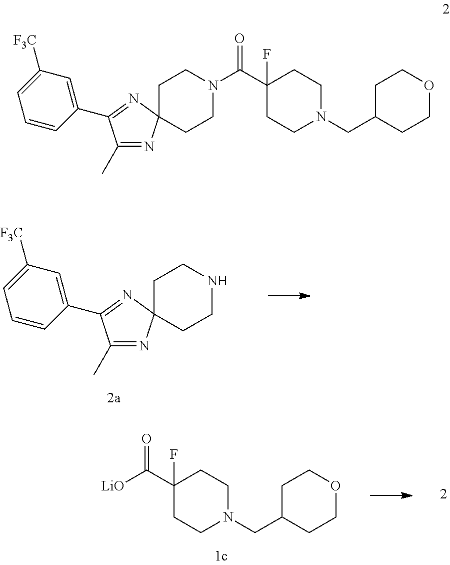

A sealed tube (15 mL) was charged with spiropiperidine 2a oxalate salt (32.1 mg, 0.083 mmol, Peakdale Fine Chemicals LTD), 1-tetrahydropyran-4-ylmethyl-piperidine-4-lithium-carboxylate 1c (27 mg, 0.108 mmol, 1.3 eq), EDC (24 mg, 0.124 mmol, 1.5 eq), BtOH (17 mg, 0.124 mmol, 1.5 eq), DIPEA (0.1 mL) and dichloromethane (2 mL). The resulting mixture was heated at 65° C. for 15 hours, then cooled to room temperature, diluted with dichloromethane (50 mL) and washed with 1 N aqueous NaOH (30 mL). The layers were separated and the aqueous was extracted with dichloromethane (2×25 mL). The combined organic phase was dried over MgSO₄, filtered and concentrated in vacuo to provide a crude oil that was purified using preparative TLC (SiO₂, dichloromethane:0.4 N NH₃ in MeOH 95:5) to provide compound 2 (21 mg, 48%) as a colorless foam. MH⁺=523

### Example 3

### Preparation of Compound 3

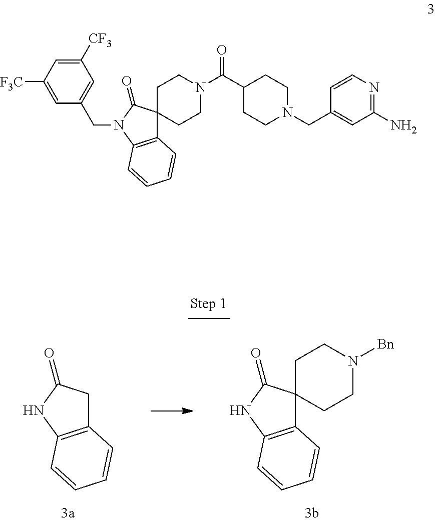

NaHMDS 1 M in THF (50 mL, 50 mmol) was added to a stirred solution of 1,3-dihydro-2H-indol-2-one 3a (1.33 g, 10 mmol) in dry THF (20 mL) at −78° C. and the mixture was stirred for 30 minutes. Then N-Benzyl-N,N-bis(2-chloroethyl)amine hydrochloride (2.68 g, 15 mmol, 1.5 eq) was added and the resulting mixture was stirred for 15 hours and slowly warmed to room temperature. The mixture was diluted with dichloromethane, then water. The organic phase was dried over MgSO₄, filtered and concentrated in vacuo to provide a residue which was purified using column chromatography (dichloromethane:MeOH 95:5) to provide compound 3b (1.7 g, 73%).

### Step 2

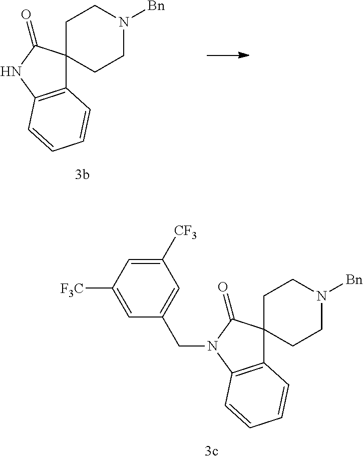

Solid KHMDS (0.78 g, 3.94 mmol, 1.6 eq) was added to a stirred solution of oxindole 3b (0.72 g, 2.46 mmol) in dry THF (12 mL) at 0° C. and the mixture was stirred for 30 minutes, during which time it was allowed to warm to room temperature. The mixture was then cooled to 0° C. and 3,5-bis(trifluoromethyl)benzyl bromide (0.54 mL, 2.95 mmol, 1.2 eq) was added dropwise and the resulting mixture was stirred for 72 hours at room temperature. The mixture was diluted with dichloromethane, then water. The organic phase was dried over MgSO₄, filtered and concentrated in vacuo to provide a residue which was purified using column chromatography (dichloromethane:MeOH 95:5) to provide compound 3c (1.145 g, 90%).

### Step 3

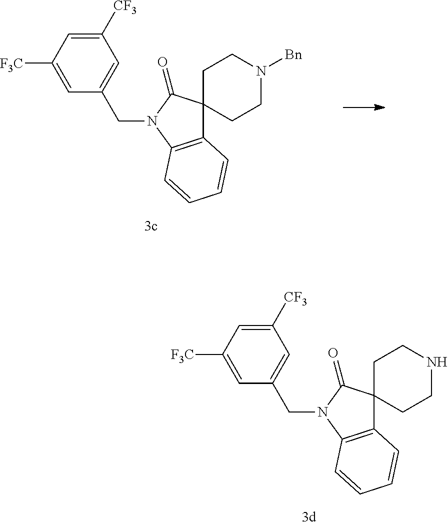

Palladium hydroxide (20% on carbon, 0.55 g) was added to a stirred solution of benzyl protected piperidine 3c (1.145 g, 2.21 mmol) in EtOH at room temperature. The resulting mixture was hydrogenated at 50 psi for 24 hours and then filtered and concentrated in vacuo. The resulting residue was redissolved in dichloromethane (6 mL), cooled to 0° C., and 4 N HCl in dioxane (2 mL) was added and the resulting mixture stirred for 1 hour at 0° C. The reaction mixture was then concentrated in vacuo to provide compound 3d.

### Step 4

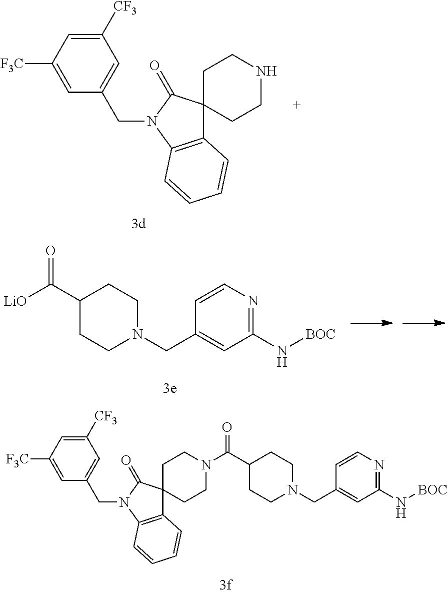

A sealed tube (15 mL) was charged with the HCl salt of compound 3d (30.5 mg, 0.066 mmol), compound 3e (29 mg, 0.085 mmol, 1.3 eq., prepared as described in International Publication No. WO 02/032893), EDC (19 mg, 0.099 mmol, 1.5 eq), BtOH (13 mg, 0.099 mmol, 1.5 eq), DIPEA (0.06 mL) and dichloromethane (2 mL). The resulting mixture was heated at 65° C. for 15 hours, then cooled to room temperature, diluted with dichloromethane (50 mL) and washed with 1 N aqueous NaOH (30 mL). The layers were separated and the aqueous was extracted with dichloromethane (2×25 mL). The combined organic phase was dried over MgSO₄, filtered and concentrated in vacuo to provide an oil that was purified using preparative TLC (SiO₂, dichloromethane:0.4 N NH₃ in MeOH 95:5) to provide compound 3f (24 mg, 48%) as a white solid.

### Step 5

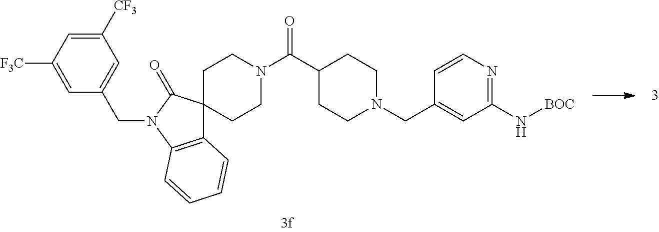

A solution of BOC-protected 2-aminopyridine 3f (24 mg, 0.031 mmol) in a mixture of dichloromethane (4 mL) and trifluoroacetic acid (1 mL) was stirred at room temperature for 20 hours. The mixture was then cooled to 0° C. and basified slowly with 10% aqueous ammonia. The resulting mixture was extracted with dichloromethane (2×25 mL) and the combined organic phase was dried over MgSO₄, filtered and concentrated in vacuo to provide compound 3 (12 mg, 57%) as a white solid. MH⁺=646

### Example 4

### Preparation of Compound 4

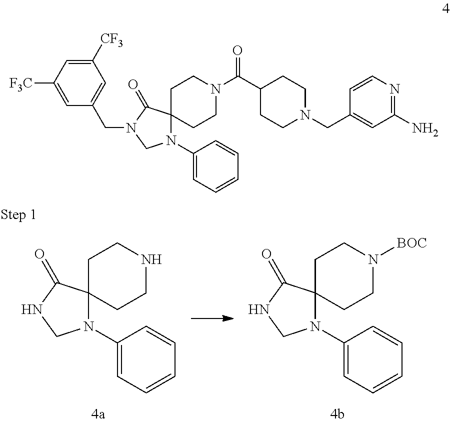

tert-Butyl dicarbonate (4.62 g, 21.2 mmol, 1.0 eq) was added to a solution of compound 4a (4.9 g, 21.2 mmol) in dichloromethane (42 mL) and the resulting mixture was stirred at room temperature for 15 hours. The mixture was diluted with dichloromethane, then water and the organic phase was dried over MgSO₄, filtered and concentrated in vacuo to provide compound 4b.

### Step 2

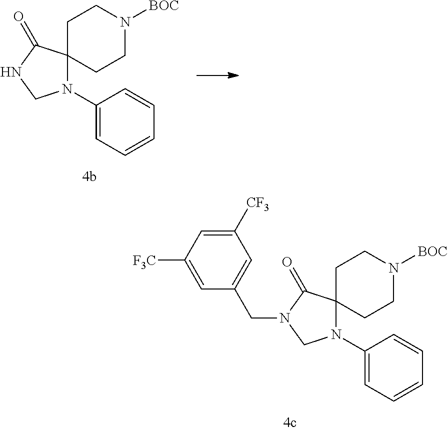

Solid KHMDS (1.41 g, 7.08 mmol, 1.6 eq) was added to a stirred solution of compound 4b (1.467 g, 4.43 mmol) in dry THF (22 mL) at 0° C. over a period of 15 minutes and the mixture was stirred for and additional 30 minutes during which time it was allowed to warm up room temperature. The reaction mixture was then cooled to 0° C. and 3,5-bis(trifluoromethyl)benzyl bromide (0.54 mL, 2.95 mmol, 1.2 eq) was added dropwise and the resulting mixture was stirred for 4 hours at room temperature. The mixture was diluted with dichloromethane, then water and the organic phase was dried over MgSO₄, filtered and concentrated in vacuo to provide a residue which was purified using column chromatography (EtOAc:Hexane 1:1) to provide compound 4c.

### Step 3

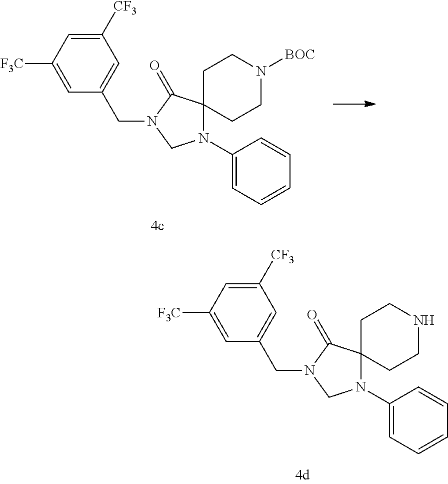

HCl (4 N in dioxane, 9 mL) was added to a stirred solution of boc-protected piperidine 4c (2.0 g, 3.59 mmol) in dichloromethane (9 mL) at 0° C. The resulting solution was stirred for 2 hours at 0° C. and during which time a precipitate formed. The crystals were filtered off and coevaporated with MeOH to provide compound 4d. Compound 4d was converted into the title compound 4 using the procedures of steps 4 and 5 of example MH⁺=675.

### Example 5

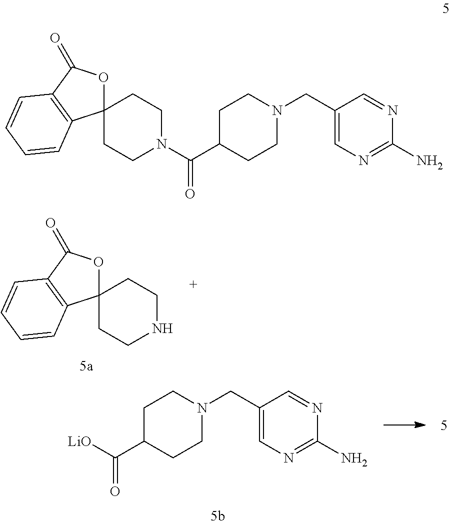

Compound 5a (commercially available from Arch Corporation) was converted to compound Sb using the methods described in International Publication No. WO 02/032893. Compound 5b was converted into the title compound 5 (MH⁺=422) using the method described in step 4 of example 3.

### Example 6

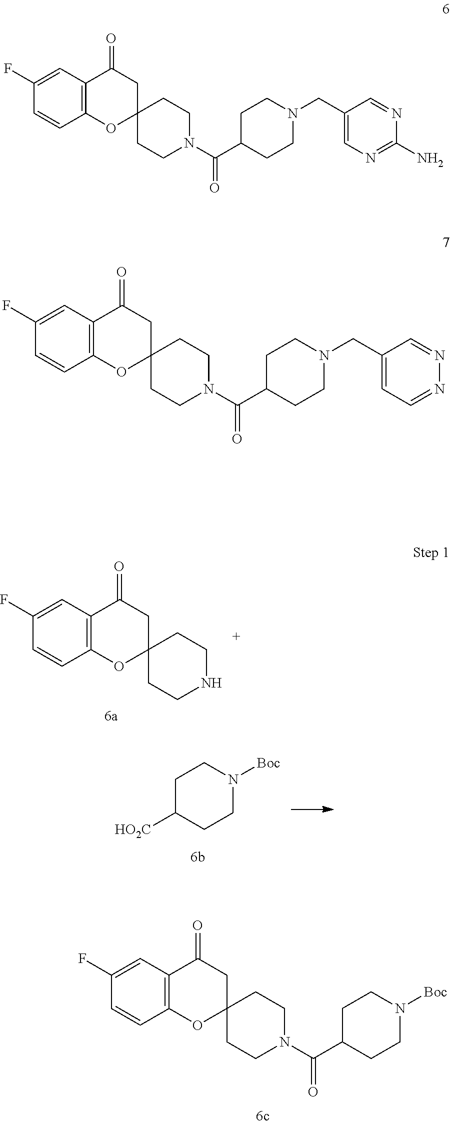

Compound 6a (commercially available from Century Labs) was converted into compound 6e using the method described in step 4 of example 3.

### Step 2

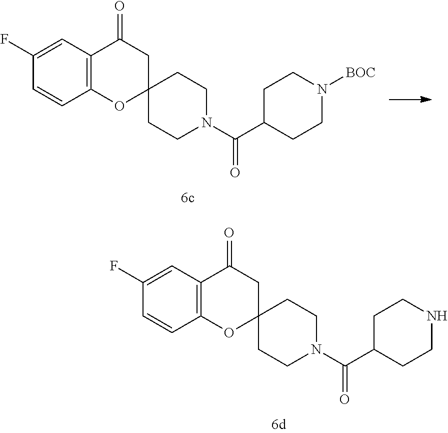

Compound 6e was converted into 6d using the method described in step 5 of example 3.

### Step 3

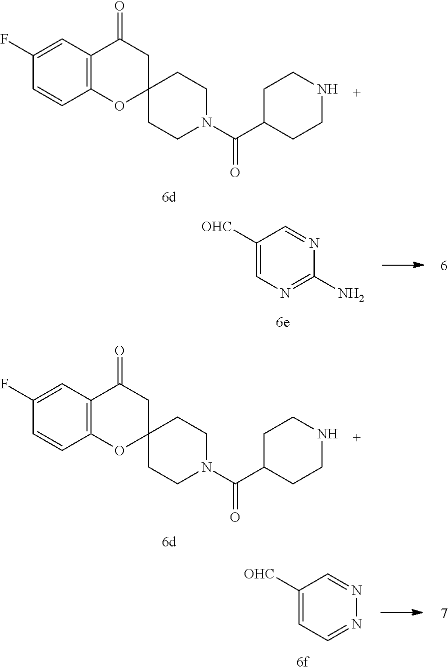

Compounds 6e and 6f were prepared and reacted with amine 6d under standard reductive amination conditions (NaBH(OAc)₃, CH₂Cl₂) as described in International Publication No. WO 02/032893 to provide compounds 6 (MH⁺=454) and 7 (MH⁺=439), respectively.

### Example 7

### Preparation of Compounds 8 and 9

To a solution of compound 6 (0.23 g, 0.51 mmol) in pyridine (8 mL) was added methoxylamine hydrochloride (0.42 g, 5.1 mmol). The reaction was heated to 60° C. and allowed to stir at this temperature for 18 hours, then the reaction mixture was cooled to room temperature and concentrated in vacuo. The crude residue obtained was purified using flash chromatography to provide 0.22 g of compound 8 as a mixture of oxime isomers (yield 89%). MH⁺=483.

Using an analogous method, compound 7 was converted to compound 9 (mixture of oxime isomers; yield 89%) MH⁺=468.

### Example 8

### Preparation of Compounds 10 and 11

### Step 1

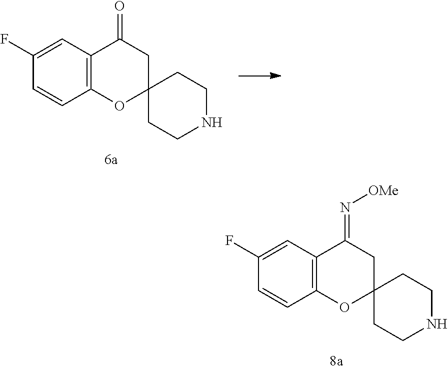

Compound 6a was converted into compound 8a (single oxime isomer) using the method described in example 7.

### Step 2

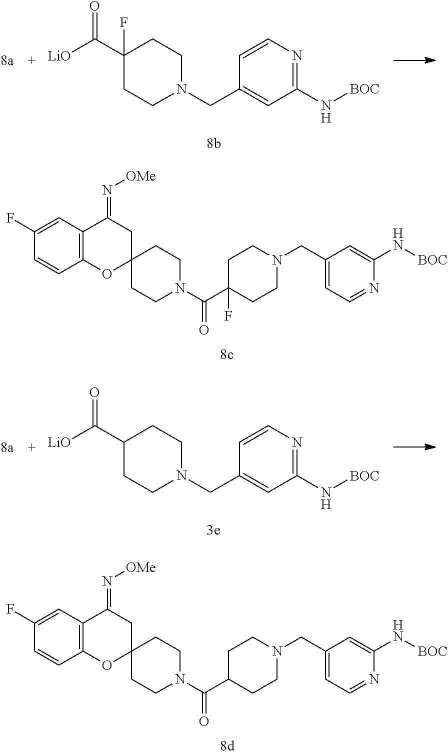

Compounds 8b and 3e were prepared as described in International Publication No. WO 02/032893.

Compound 8c was prepared from 8a and 8b using the method described in step 4 of example 3.

Compound 8d was prepared from 8a and 3e using the method described in step 4 of example 3.

### Step 3

Compounds 8c and 8d were converted into compounds 10 (single oxime isomer, MH⁺=500) and 11 (single oxime isomer, MH⁺=482), respectively, using the method described in step 5 of example 3.

### Example 9

### Preparation of Compound 12

### Step 1

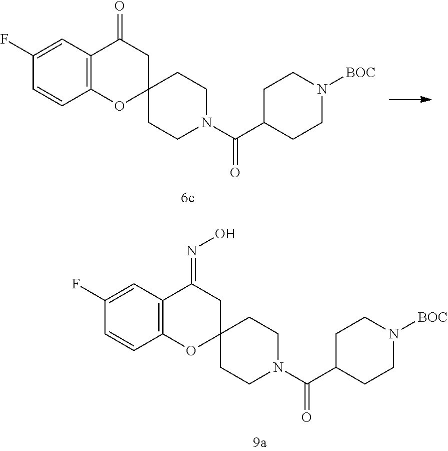

Compound 6c was converted into compound 9a using the method described in example 7.

### Step 2

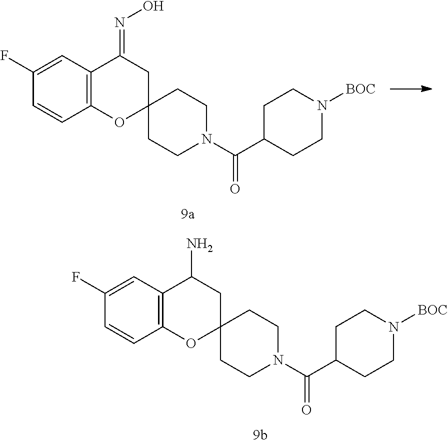

To a 500 mL hydrogenation bottle was added compound 9a (1.17 g), MeOH (30 mL), and Raney Nickel (2 g). The mixture was hydrogenated at 50 Psi for 72 hours, then filtered through celite, and the filtrate was concentrated in vacuo to provide compound 9b (yield 94%).

### Step 3

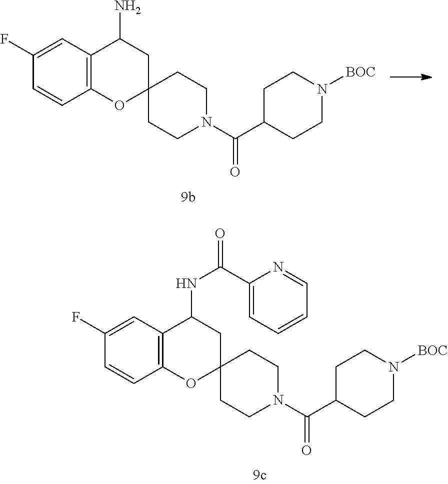

To a solution of compound 9a (0.20 g, 0.45 mmol) in CH₂Cl₂ (8 mL) was added EDC (0.13 g, 0.67 mmol), HOBT (0.091 g, 0.67 mmol), and picolinic acid (0.06 g, 0.45 mmol). After stirring at room temperature for 20 hours, the mixture was extracted with CH₂Cl₂ and 5% aqueous NaOH solution, dried over Na₂SO₄, filtered, concentrated in vacuo, and purified using flash chromatography to provide compound 9c (0.27 g, 94%).

### Step 4

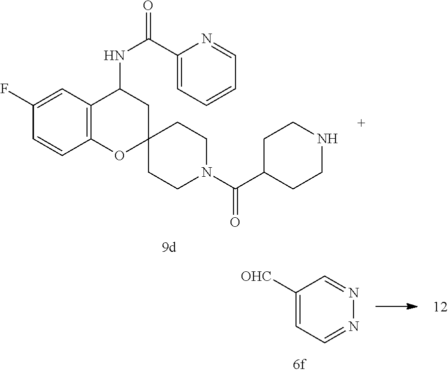

Compound 9d was prepared from compound 9c using a procedure analogous to the that described in step 5 of example 3.

Compound 6f was prepared and reacted with amine 9d (prepared from compound 9c using a procedure analogous to the that described in step 5 of example 3) using the reductive amination conditions (NaBH(OAc)₃, CH₂Cl₂) described in International Publication No. WO 02/032893 to provide compound 12. MH⁺=545

### Example 10

### Preparation of Compound 13

### Step 1

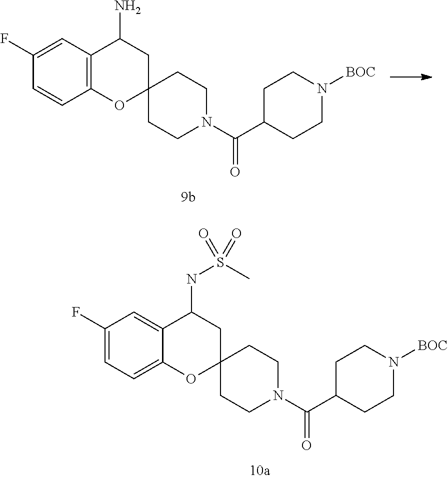

To a 0° C. solution of compound 9b (0.3 g, 0.67 mmol) and Et₃N (0.28 mL, 2.01 mmol) in CH₂Cl₂ (5 mL) was slowly added ClSO₂CH₃ (0.15 g, 1.34 mmol). The resulting reaction was allowed to stir at 0° C. for 1 hour, then at room temperature for another hour. The reaction mixture was then extracted with CH₂Cl₂ and H₂O, dried over Na₂SO₄, filtered, concentrated in vacuo, and the residue obtained was purified using flash chromatography to provide compound 10a (0.3 g, 97%).

### Step 2

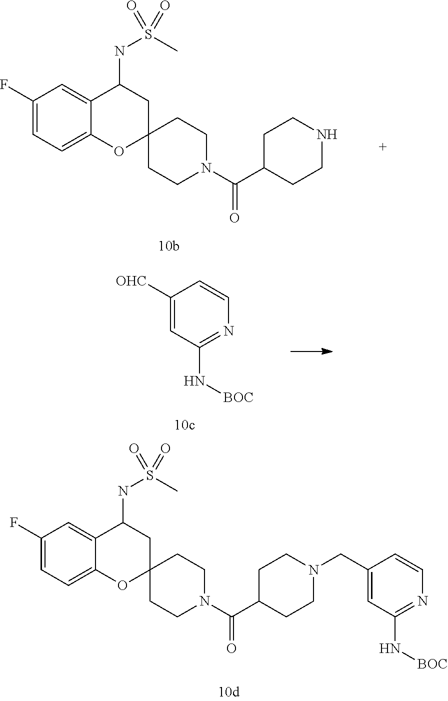

### Step 3

Compound 10c was prepared and reacted with amine 10b prepared from compound 10a using procedure analogous to the one described in step 5 of example 3) using the reductive amination conditions (NaBH(OAc)₃, CH₂Cl₂) described in International Publication No. WO 02/032893 to provide 10d.

### Step 4

Compound 10d was converted into the title compound 13 (MH⁺=532) using the method describe in step 5 of example 3.

### Example 11

### Preparation of Compound 14

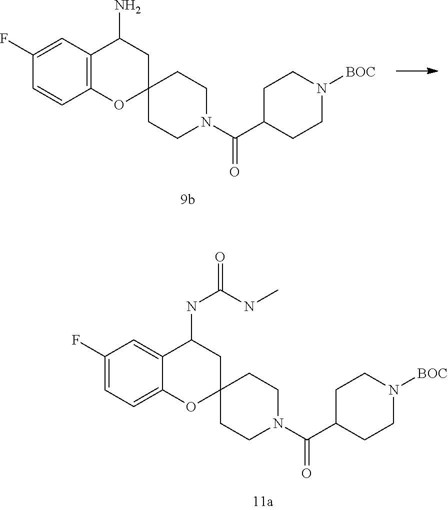

### Step 1

To a solution of compound 9b (036 g, 0.80 mmol) in 10 ml of THF was added methyl isocyanate (0.092 g, 1.6 mmol). After stirring at room temperature for 20 h, the mixture was extracted with EtOAc and saturated NaHCO₃, dried over Na₂SO₄, filtered, concentrated, and purified using flash chromatography to provide compound 11a, yield 0.35 g (99%).

### Step 2

Compound 11a was converted into the title compound 14 (MH⁺=511) using the method described in example 10.

### Example 12

### Preparation of Compounds 15 and 16

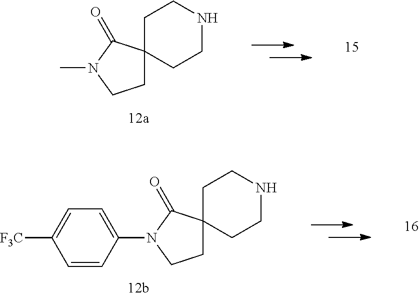

Compounds 12a and 12b (each commercially available from Arch Corporation) were converted into compounds 15 (MH⁺=386) and 16 (MH⁺=516), respectively, by using methods analogous to those described in steps 4 and 5 of example 3.

### Example 13

### Preparation of Compound 17

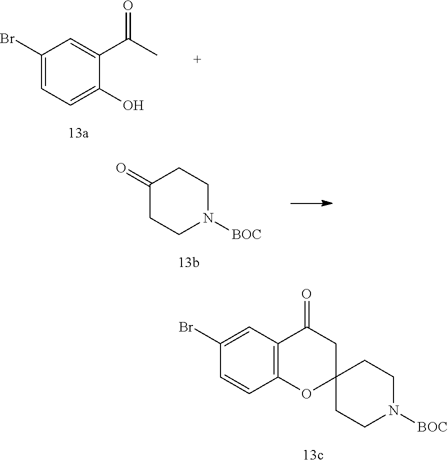

### Step 1

A mixture of compound 13a (11.1 g, 52 mmol) and pyrrolidine (5.6 mL, 67 mmol) in toluene (200 mL) was stirred at 20° C. for 20 minutes. The mixture was then treated with 1-BOC-4-piperidone 13b (13.4 g, 67 mmol) and refluxed for about 15 hours. The reaction mixture was then cooled to room temperature, washed sequentially with 10% aqueous NaOH and H₂O, dried over MgSO₄, filtered and concentrated in vacuo. The residue obtained was purified using flash column chromatography (10% EtOAc/hexanes) to provide compound 13c (20.0 g, 97%) as a yellow solid.

### Step 2

Compound 13c was converted to compound 17 (MH⁺=513) using the methods described in steps 4 and 5 of example 3.

### Example 14

### Preparation of Compound 18

Compound 18 (mixture of oxime isomers, MH⁺=542) was prepared from compound 17 using the method described in example 7.

### Example 15

### Preparation of Compound 19

### Step 1

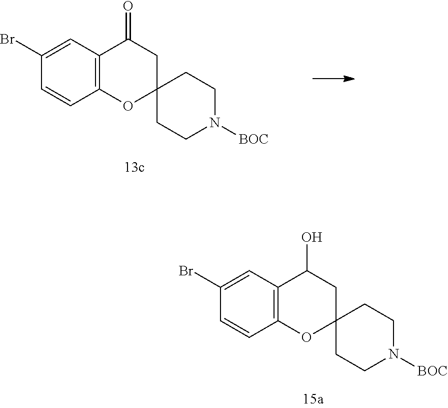

A solution of compound 13c (1.55 g, 3.9 mmol) in MeOH (30 mL) was treated with NaBH₄ (0.18 g, 4.7 mmol) and the resulting reaction was allowed to stirred at 20° C. for about 15 hours, then concentrated in vacuo. The resulting residue was taken up in CH₂Cl₂, washed with saturated aqueous NaHCO₃, dried over Na₂SO₄, filtered and concentrated in vacuo. The residue obtained was purified using flash column chromatography (30% EtOAc/hexanes) to provide compound 15a (1.29 g, 83%) as a white solid.

### Step 2

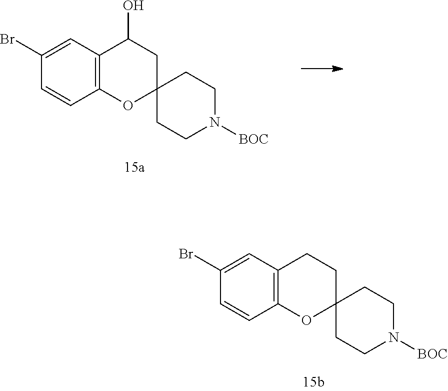

A solution of 15a (0.88 g, 2.2 mmol) in CH₂Cl₂ (3 mL) was treated sequentially with TFA (20 mL) and then Et₃SiH (3.2 mL, 20 mmol). The reaction was stirred at 20° C. for 3 days, neutralized with 10% aqueous NaOH, and extracted with CH₂Cl₂ (2×). The combined organic layers were dried over Na₂SO₄, filtered and concentrated in vacuo. The residue obtained was subjected to flash column chromatography (60% EtOAc/hexanes) to provide compound 15b (0.45 g, 72%) as a white oil.

### Step 3

Compound 15b was converted to compound 19 (MH⁺=499) using the method described in example 10.

### Example 16

### Preparation of Compound 20

### Step 1

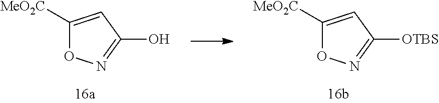

Compound 16a (2.5 g, 17.47 mmol) was dissolved in 100 mL of CH₂Cl₂ and to the resulting solution was added N,N′-dimethylaminopyridine (DMAP, 2.77 g, 22.67 mmol) and chloro-tert-butyldimethylsilane (3.29 g, 21.83 mmol). The resulting reaction was allowed to stir for about 15 hours, then was diluted with 100 mL of CH₂Cl₂, washed with a saturated NaHCO₃ aqueous solution and brine and dried over Na₂SO₄. The dried solution was then filtered through a one-inch silica gel pad, and the pad was rinsed with CH₂Cl₂. The filtrate was concentrated in vacuo to provide 3.96 g (88%) of compound 16b as a colorless oil, which was used without further purification.

### Step 2

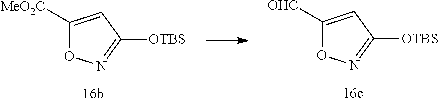

To a stirred solution of compound 16b (3.95 g, 15.35 mmol) in 100 mL of CH₂Cl₂ at −78° C. was added dropwise a 1.0 M solution of diisbutylaluminum hydride in CH₂Cl₂ over a 30 minute period. The resulting reaction was allowed to stir for 2 hours, then quenched with saturated aqueous NH₄Cl solution. The quenched reaction was then neutralized using 1.0 M HCl aqueous solution and allowed to sit for 5 minutes. The solution was separated and the aqueous layer was extracted with CH₂Cl₂ (100 mL×2). The combined organic layers were washed with brine, dried over Na₂SO₄, and filtered through a 1-in silica gel pad. The filtrate was concentrated in vacuo to provide 2.46 g of aldehyde 16c (70%) as a colorless oil (containing small amount of the alcohol by-product), which was used without further purification.

### Step 3

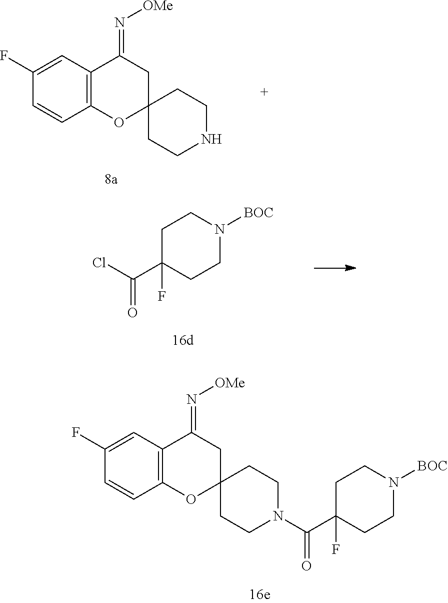

Compound 16d (prepared as described in as described in International Publication No. WO 02/032893) and reacted with compound 8a using the method described in step 4 of example 30 below.

### Step 4

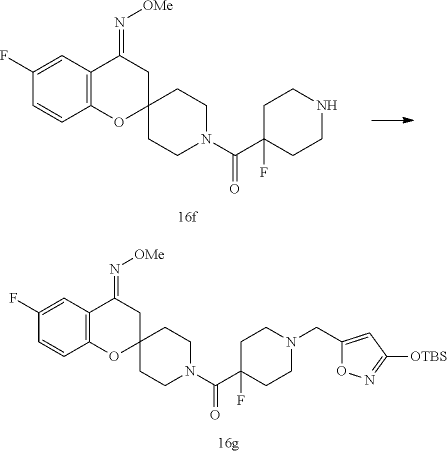

To a stirred solution of compound 16f (75 mg, 0.192 mmol, prepared from compound 16e using the method described in step 5 of example 3) in 3 mL of CH₂Cl₂ at room temperature was added compound 16c (65 mg, 0.286 mmol). To the resulting solution was added sodium triacetoxyborohydride (61 mg, 0.288 mmol), followed by two drops of acetic acid. The resulting reaction was stirred for about 15 hours, and H₂O was added. The aqueous mixture was extracted with CH₂Cl₂ (20 mL×3). The organic extracts were washed with H₂O and brine, dried over Na₂SO₄, and concentrated in vacuo to provide a crude yellow oil, which was purified using preparative TLC (CH₂Cl₂-MeOH=25:1, v/v) to provide 38 mg of compound 16g (33%) as a colorless solid.

### Step 5

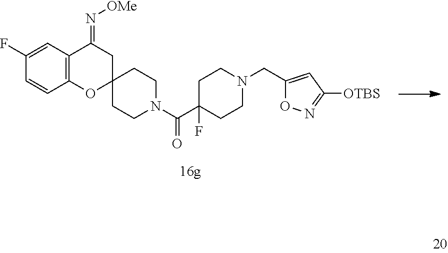

Compound 16g (38 mg, 0.063 mmol) was dissolved in 2 mL of THF and to the resulting solution was added a 1.0 M solution of tetrabutylammonium fluoride in THF. The resulting reaction was allowed to stir for about 15 hours, then water was added. The resulting solution was extracted with CH₂Cl₂ (20 mL×3) and the combined organic extracts were washed with brine, dried over Na₂SO₄, and concentrated in vacuo to provide an oil material, which was purified using preparative TLC (CH₂Cl₂-MeOH=15:1, v/v) to provide 17.5 mg of compound 20 as a colorless solid (57%). MH⁺=491

### Example 17

### Preparation of Compound 21

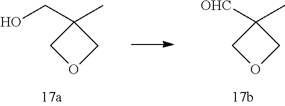

### Step 1

Compound 17a (1.11 g, 10.87 mmol) was dissolved in 50 mL of CH₂Cl₂ and to the resulting reaction was added Dess-Martin periodinane (5.76 g, 13.59 mmol) in one portion.

The resulting reaction was allowed to stir for 5 hours, then was quenched with 50 mL of a 1.0 M NaOH aqueous solution. The quenched solution was diluted with 150 mL of diethyl ether and separated. The organic layer was washed with H₂O and brine, dried with MgSO₄, filtered, and concentrated in vacuo to provide 0.71 g of aldehyde 17b which was used without further purification.

### Step 2

Compound 17b was converted to compound 21 using the method described in step 4 of example 16. MH⁺=478

### Example 18

### Preparation of Compound 22

### Step 1

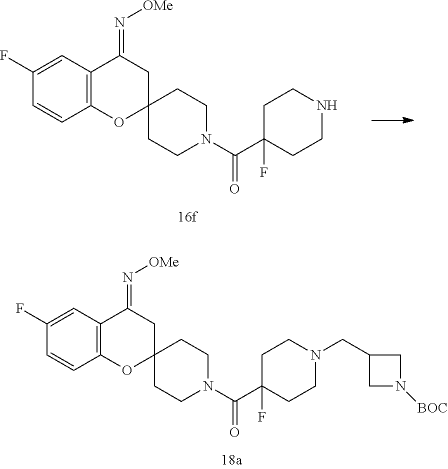

Compound 18a was prepared by reacting compound 16f (69 mg, 0.175 mmol) and 1-BOC-3-formylazetidine (95 mg, 0.512 mmol) according to the method described in step 4 of example 16. Crude yield 138 mg.

### Step 2

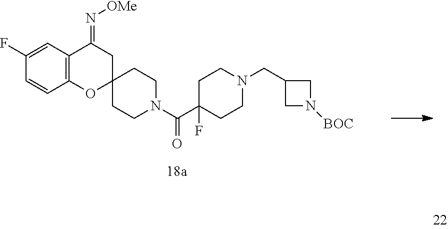

Compound 18a (138 mg, from step 1) was dissolved in 3 mL of CH₂Cl₂ and to the resulting solution was added trifluoroacetic acid (0.7 mL) and the reaction was stirred at room temperature for 2.5 hours. The reaction mixture was concentrated in vacuo, and the oily residue was dissolved in 75 mL of CH₂Cl₂. The resulting solution was washed with a saturated NaHCO₃ aqueous solution (2×), then brine, dried over Na₂SO₄, and concentrated in vacuo. The oily residue obtained was purified using preparative TLC(CH₂Cl₂-7M NH₃ in MeOH=15:1, v/v) to provide compound 22 as a creamy yellow solid (31 mg, 38% over two steps). MH⁺=463

### Example 19

### Preparation of Compound 23

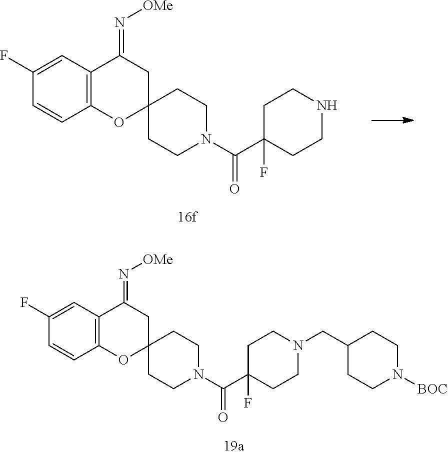

Compound 19a was prepared from compound 16f (described in step 4 of example 16) and 1-BOC-4-piperidinecarboxaldehyde using procedure described in step 4 of example 16.

Compound 19a was converted into the title compound 23 using procedure described in step 2 of example 18. MH⁺=491

### Example 20

### Preparation of Compound 24

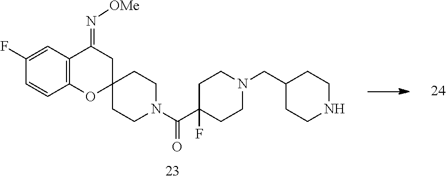

To a stirred solution of compound 23 (219 mg, 0.592 mmol) in 30 mL of CH₂Cl₂ at room temperature was added aqueous formaldehyde (37% w in H₂O, 0.072 ml, 0.89 mmol). Sodium triacetoxyborohydride (30 mg, 0.89 mmol) was added followed by three drops of acetic acid. The resulting mixture was stirred for about 15 hours, and quenched with a saturated NaHCO₃ aqueous solution. The mixture was extracted with CH₂Cl₂. The organic extracts were washed with brine, dried over Na₂SO₄, and concentrated in vacuo to a yellow oil, purified using preparative TLC (6% of a 7M methanolic ammonia solution in CH₂Cl₂) to provide 160 mg of compound 24 (73%). MH⁺=505

### Example 21

### Preparation of Compound 25

### Step 1

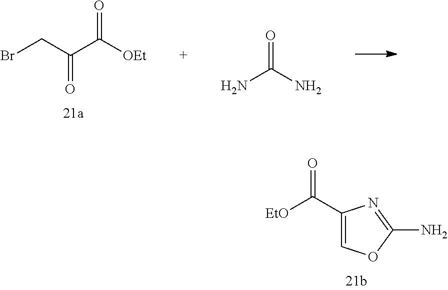

Following the procedure, described in J. Med. Chem. 1971, 14, 1075-1077, ethyl bromopyruvate 21a was condensed with urea in ethanol under reflux to provide ethyl-2-amino-4-oxazole carboxylate 21b.

### Step 2

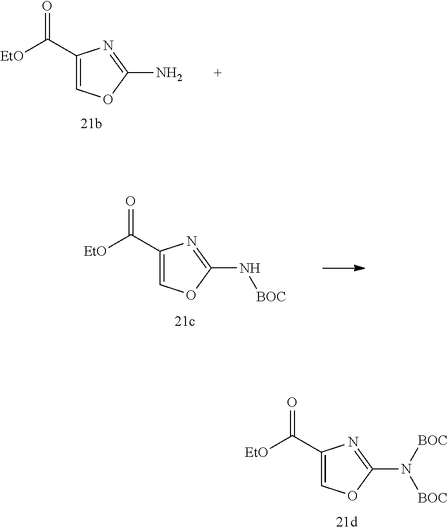

Aminooxazole carboxylate 21b (1.79 g, 11.46 mmol) was dissolved in 60 mL of CH₂Cl₂. 4-N,N′-dimethylamino pyridine (1.54 g, 12.61 mmol) and di-cert-butyl dicarbonate (2.76 g, 12.62 mmol) were added. The mixture was stirred at room temperature for 20 hours, quenched with a saturated NaHCO₃ aqueous solution. The aqueous mixture was extracted with CH₂Cl₂ (50 mL×3). The organic extracts were washed with a 0.5 M HCl aqueous solution and brine, dried over Na₂SO₄, and concentrated in vacuo to provide 2.43 g of Boc-protected products 21c and 21d (21c:21d˜1:3).

### Step 3

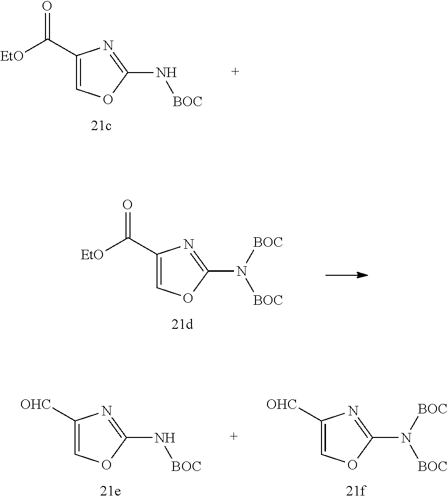

Mixture of compounds 21c and 21d from step 3 (2.42 g, 7.28 mmol) was dissolved in 70 mL of CH₂Cl₂ and cooled in a −78° C. bath. A 1.0 M solution of diisobuylaluminum hydride in CH₂Cl₂ (9.5 mL, 0.5 mmol) was added dropwise during a 20 min period. Reaction was continued for 1 hour, and quenched with 35 mL of a saturated NH₄Cl aqueous solution and 35 mL of a 1.0 M HCl aqueous solution. The layers were separated. The aqueous layer was extracted with CH₂Cl₂ (35 mL), and the combined organic layeres were washed with brine, dried over Na₂SO₄, and concentrated in vacuo to pale yellow oil, which was separated by preparative TLC, eluting with CH₂Cl₂-MeOH (25:1, v/v) to provide 0.26 g of mono-boc protected amino aldehyde 21e and 0.38 g of bis-boc protected amino aldehyde 21f.

Mixture of compounds 21e and 21f was converted into the title compound 25 (MH⁺=490) following the procedures described in example 18.

### Example 22

### Preparation of Compound 26

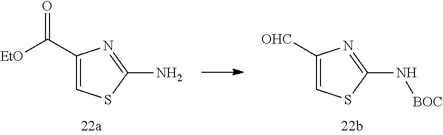

Ethyl-2-amino-4-thiazolecarboxylate 22a was converted into aldehyde 22b using the procedures of steps 2 and 3 of example 21.

Compound 22b was converted into the title compound 26 (MH⁺=506) following the procedures described in example 18.

### Example 23

### Preparation of Compound 27

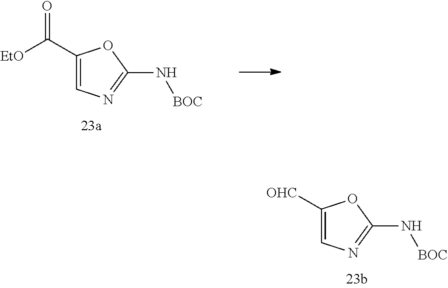

Compound 23a was prepared from ethyl-2-amino-oxazole-5-carboxylate using the procedure from step 2 of example 21.

Compound 23a (0.60 g, 235 mmol) was dissolved in 25 mL of CH₂Cl₂ and cooled in a −78° C. bath. A 1.0 M solution of diisobutylaluminum hydride in CH₂Cl₂ was added dropwise along the inside wall of the flask. Reaction was continued for 1.5 hours. A saturated NH₄Cl aqueous solution was added, followed by a 1.0 M HCl aqueous solution. The mixture was extracted with CH₂Cl₂ (75 mL×3). The organic extracts were washed with brine, dried over Na₂SO₄, and concentrated in vacuo to pale yellow oil, which was separated by preparative TLC (CH₂Cl₂-MeOH=40:1, v/v) to provide 45 mg of the unreacted starting material 23a and 124 mg of aldehyde 23b (25%) as an off-white solid.

Compound 23b was converted into the title compound 27 (MH⁺=490) following the procedures described in example 18.

### Example 24

### Preparation of Compound 28

### Step 1

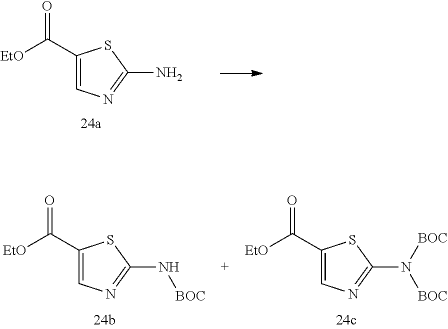

Compound 24a (5 g, 31.6 mmol) was dissolved in 200 mL of CH₂Cl₂ and to the resulting solution was added 4-N,N′-dimethylamino pyridine (5.8 g, 47.48 mmol) and di-tert-butyl dicarbonate (10.0 g, 45.82 mmol). The resulting reaction was stirred for about 15 hours, filtered through a fritted funnel and rinsed with CH₂Cl₂. The filtrate was washed sequentially with a saturated NaHCO₃ aqueous solution (50 mL), H₂O (50 mL), a 1.0 M HCl aqueous solution (50 mL), and brine (50 mL). The organic solution was dried over Na₂SO₄, filtered, and concentrated in vacuo to provide a yellow solid residue which was treated with 200 mL of hexanes and filtered. The collected solid material was washed with 100 mL of hexanes and dried under vacuum to provide 5.26 g of mono-boc protected product 24b (65%). The hexanes filtrate and washing were combined, and concentrated in vacuo to provide 1.95 g of the bis-boc protected product 24c (17%) as a light yellow solid.

### Step 2

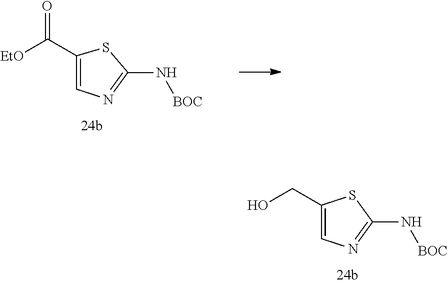

Compound 24b (5.26 g, 20.36 mmol) was dissolved in 150 mL of CH₂Cl₂ and cooled to −78° C. A 1.0 M solution of dissobutyl aluminum hydride in CH₂Cl₂ (56 mL, 56 mmol) was added dropwise and the resulting reaction was allowed to stir for about 15 hours while gradually warming to room temperature. A saturated NH₄Cl aqueous solution was added and the resulting solution was stirred for 3 hours, then filtered through a Celite pad, which was subsequently rinsed with CH₂Cb and H₂O. The two layers of the filtrate were separated, and the aqueous layer was extracted with CH₂Cl₂ (2×). The combined organic extracts were washed with brine, dried over Na₂SO₄, and concentrated in vacuo to provide a light yellow solid residue which was purified using flash column chromatography (CH₂Cl₂-MeOH (150:1, 100:1, and 50:1, v/v)) to provide compound 24d (1.98 g, 42%) as a near colorless solid.

### Step 3

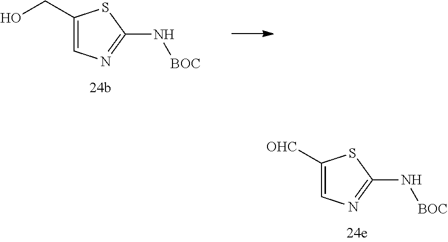

### Step 4

Compound 24d (1.87 g, 8.12 mmol) was dissolved in 100 mL of CH₂Cl₂ and 3 mL of ethyl acetate and to the resulting solution was added manganese (IV) dioxide (8.5 g, 117.3 mmol). The resulting reaction was allowed to stir at room temperature for 14 hours, then was filtered through a Celite pad, which was rinsed with CH₂Cl₂ and ethyl acetate. The filtrate was concentrated in vacuo to provide 1.8 g of the aldehyde 24e (92%) as a creamy yellow solid, which was used without further purification.

### Step 5

Compound 24e was converted to compound 28 (MH⁺=506) using the method described in example 18.

### Example 25

### Preparation of Compound 29

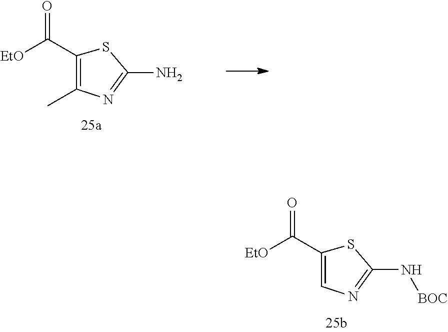

### Step 1

Compound 25a (4.6 g, 20.66 mmol) was suspended in 100 mL of CH₂Cl₂. 4-N,N′-dimethylamino pyridine (3.2 g, 26.19 mmol) and to the resulting suspension was added ditertbutyl Bicarbonate (5.64 g, 25.84 mmol). The resulting reaction was allowed to stir for 2 days, then was diluted with 100 mL of CH₂Cl₂, washed sequentially with a saturated NaHCO₃ aqueous solution (50 mL), H₂O (50 mL), a 1.0 M HCl aqueous solution (50 mL), H₂O (50 mL), and brine (50 mL) The organic solution was dried over Na₂SO₄, filtered, and concentrated in vacuo to provide 4.02 g of the mono-boc protected product 25b (60%) as a colorless solid.

### Step 2

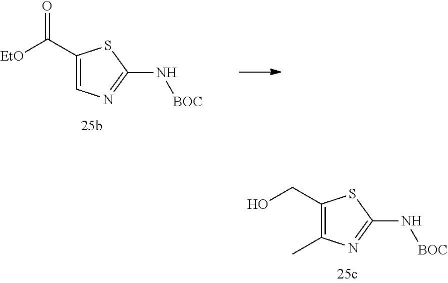

Compound 25b (4.02 g, 12.45 mmol) was dissolved in 100 mL of CH₂Cl₂ and cooled in a −78° C. bath and to the cooled solution was added dropwise a 1.0 M solution of diisobutylaluminum hydride in CH₂Cl₂ (40 mL, 40 mmol). The cool bath was removed and the resulting reaction was allowed to stir for 24 hours while warming to room temperature. The reaction mixture was poured carefully into a well stirred mixture of 300 mL of a saturated NH₄Cl aqueous solution and 50 mL of ice. Stirring was continued for 3 hours, then the resulting mixture was filtered through a Celite pad and rinsing with CH₂Cl₂. The two layers of the filtrate were separated, and the aqueous layer was extracted with CH₂Cl₂ (100 mL×3). The combined organic extracts were washed with brine, dried over Na₂SO₄, and concentrated in vacuo to provide 3.01 g of compound 25c (100%) as a colorless solid, which was used without further purification.

### Step 3

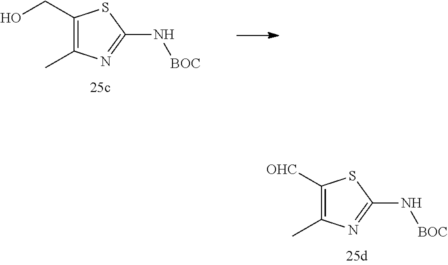

Compound 25c (3 g, 12.45 mmol), was dissolved in 100 mL of CH₂Cl₂ and to the resulting solution was added Dess-Martin periodinane (7.13 g, 16.81 mmol). The resulting reaction was allowed to stir at room temperature for 3.5 hours, then a 1.0 M NaOH aqueous solution was added (100 mL). The resulting mixture was extracted with CH₂Cl₂ (50 mL×3), and the basic aqueous solution was neutralized with a 1.0 M HCl aqueous solution, then extracted with CH₂Cl₂ (75 mL×5). The combined organic extracts were washed with brine, dried over Na₂SO₄, and concentrated in vacuo to provide a light yellow crude solid. The crude solid was extracted with hexanes—CH₂Cl₂ (10:1), filtered, and the filtrate was concentrated in vacuo to provide 0.9 g of compound 25d (30%) as a near colorless solid.

### Step 4

Compound 25d was converted to compound 29 (MH⁺=520) using the methods described in example 18.

### Example 26

### Preparation of Compound 30

### Step 1

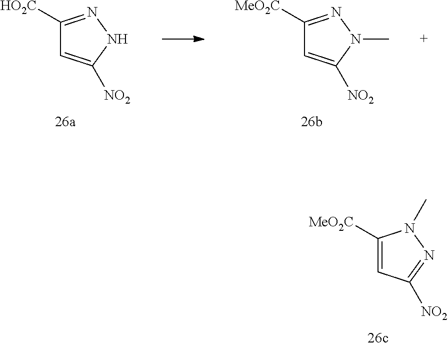

To a stirred suspension of compound 26a (1.0 g, 6.41 mmol) in 60 mL of THF was added lithium methoxide (0.365 g, 9.61 mmol). After 1 hour, iodornethane (0.96 mL, 15.42 mmol) was added. The reaction was allowed to continue at room temperature for 2.5 days and was then filtered through a 1-in silica gel pad and rinsed with EtOAc. The combined filtrate and rinsing were concentrated to a yellow oil, which was triturated with CH₂Cl₂— hexanes (˜1:1) and filtered. The filtrate was concentrated in vacuo to afford 1.03 g of a 1:1 ratio mixture of 26b/26c as an off-white solid (87%). The material was used without further purification.

### Step 2

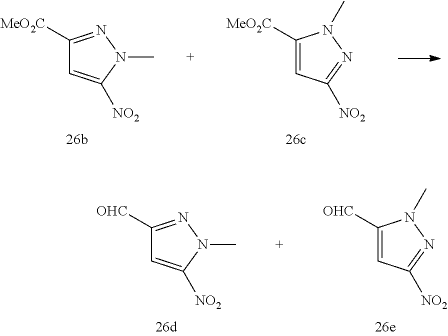

Using the method described in example 23, a mixture of esters 26b and 26c (0.623 g, 3.36 mmol) was reduced to the corresponding aldehydes 26d (0.208 g, 40%) and 26e (0.137 g, 26%).

### Step 3

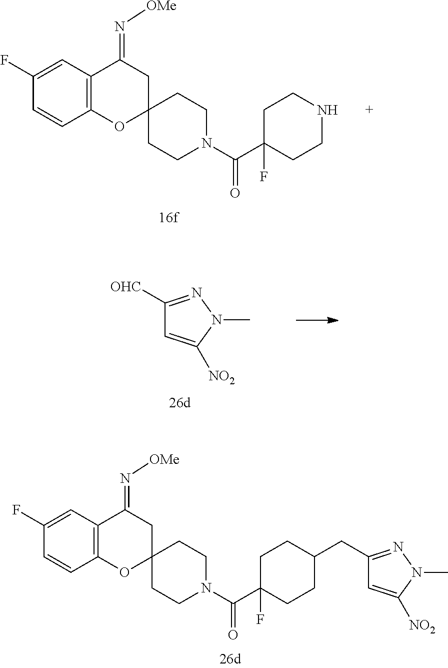

Compound 16f was reacted with compound 26d using the method described in Example 18 to provide compound 26f.

### Step 4

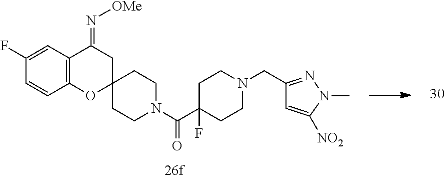

Compound 26f (176 mg, 0.33 mmol) was dissolved in 10 mL of ethanol and the resulting solution was degassed, put under nitrogen atmosphere, then 10% palladium on carbon (88 mg, wet with 50% H₂O) was added. The resulting solution was again degassed, and placed under hydrogen atmosphere (using a H₂-filled balloon) and the resulting reaction was allowed to stir for about 15 hours. The reaction mixture was then filtered through a Celite pad, which was rinsed with ethyl acetate. The combined filtrates were concentrated in vacuo to provide a crude oil, which was purified using preparative TLC (CH2Cl2-7N NH₃ in MeOH=30:1, v/v) to provide compound 30 (110 mg, 66%) MH⁺=503.

### Example 27

### Preparation of Compound 31

### Step 1

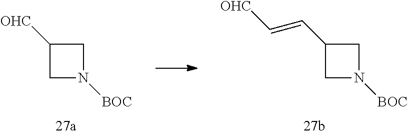

3-Formylazetidine-1-carboxylic acid tert-butyl ester 27a (0.966 g, 5.20 mmol) was dissolved in 25 mL of THF and the resulting solution was cooled to 0° C. Triphenylphosphoranylidine aldehyde (1.98 g, 6.51 mmol) was added and after 1 hour cooling bath was removed, and the reaction was allowed to stir at room temperature for 2 days. The reaction mixture was then filtered through a silica gel pad, which was rinsed with CH₂Cl₂ and the filtrate was concentrated in vacuo to provide a crude yellow oil, which was purified using MPLC (Biotage, 40+M cartridge, eluted with CH₂Cl₂, CH₂Cl₂-MeOH (400:1, 200:1, 100:1, v/v)) to provide compound 27b as pale yellow oil (0.69 g, 63%).

### Step 2

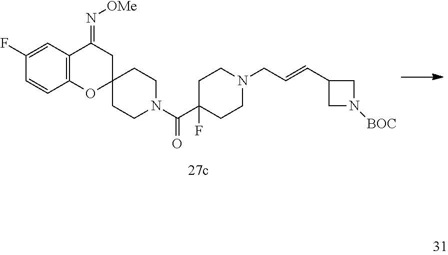

Compound 27e (73 mg, 0.124 mmol, prepared from compound 27b using the method described in example 18) was dissolved in 2 mL of CH₂Cl₂ and cooled to −78° C. To the cooled solution was added iodotrimethylsilane (0.025 mL, 0.176 mmol) dropwise. The resulting reaction was stirred for 5 hours during which time the reaction was allowed to warm to 0° C. and maintained at this temperature. A 1.0 M NaOH aqueous solution (10 mL) and methanol (2 mL) were then added and the resulting aqueous mixture was extracted with CH₂Cl₂ (20 mL) and ethyl acetate (20 mL×3). The combined organic extracts were washed with brine, dried over Na₂SO₄, and concentrated in vacuo to provide compound 31 (82 mg, 100%) which was purified using preparative TLC (CH₂Cl₂-7N NH₃ in MeOH=15:1 v/v, 2 elutions, 40% purified yield). MH⁺=489

### Example 28

### Preparation of Compound 32

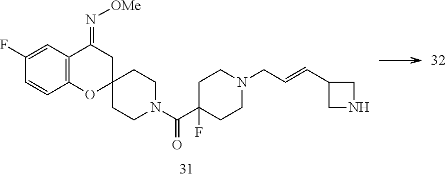

Compound 31 (82 mg) was dissolved in 3 mL of CH₂Cl₂ and to the resulting solution was added a 37% aqueous solution of formaldehyde (0.2 ml, 2.68 mmol), followed by sodium triacetoxyborohydride (112 mg, 0.529 mmol) and two drops of acetic acid. The resulting reaction was allowed to stir for about 15 hours, then a 1.0 M NaOH aqueous solution was added, followed by water. The resulting aqueous mixture was extracted with CH₂Cl₂ (20 mL×3) and the combined organic extracts were washed with brine, dried over Na₂SO₄, and concentrated in vacuo to provide a crude oil which was purified using preparative TLC (CH₂Cl₂-7N NH₃ in MeOH=15:1, v/v) to provide compound 32 (51 mg, 58%) MH⁺=503

### Example 29

### Preparation of Compound 33

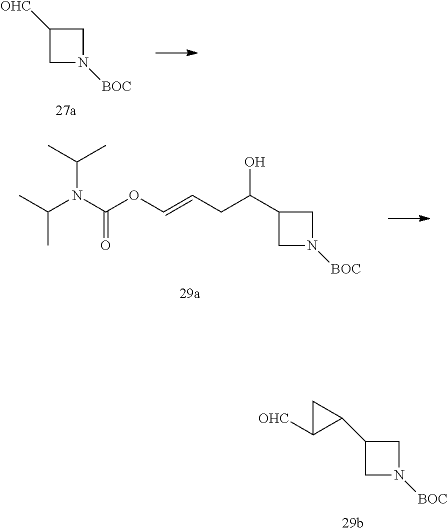

### Step 1

Compound 27a (0.7 g, 3.77 mmol) was converted to compound 29b (0.33 g, 39%) as described in Org. Lett. 2003, 5, 1377-1379.

### Step 2

Compound 29b was converted to compound 33 using the methods described in examples 27 and 28. MH⁺=517

### Example 30

### Step 1

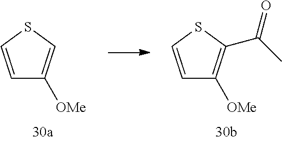

Compound 30a (3.5 g, 30.66 mmol) was dissolved in 100 mL of CH₂Cl₂ and the resulting solution was cooled to 0° C. Acetyl chloride (3.3 mL, 78.50 mmol) was added followed by aluminum chloride (4.1 g, 30.75 mmol) and the resulting reaction was allowed to stir for 4 hours at 0° C. The reaction mixture was poured into 200 mL of crushed ice and transferred to a separatory funnel once the ice melted. The aqueous layer was extracted with CH₂Cl₂ (100 mL×2), and the combined organic extracts were washed with H₂O and brine, then dried over Na₂SO₄ and filtered through a short silica gel pad. The filtrate was concentrated in vacuo to provide compound 30b (4.1 g, 85%), which was used without further purification.

### Step 2

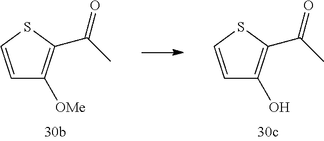

Compound 30b (4.10 g, 26.25 mmol) was dissolved in 100 mL of CH₂Cl₂ and the resulting solution was cooled to −78° C. To the cooled solution was added dropwise a 1.0 M solution of boron tribromide in CH₂Cl₂ (33 mL, 33 mmol). The cold bath was removed and the resulting reaction was allowed to stir for 7 hours while the reaction temperature gradually increased to room temperature. Water was then added and the aqueous layer was extracted with CH₂Cl₂ (100 mL×2). The combined organic layers were washed with H₂O and brine, dried over Na₂SO₄, and filtered through a Celite pad. The filtrate was concentrated in vacuo to provide compound 30c (3.56 g, 95%) as a dark yellow oil.

### Step 3

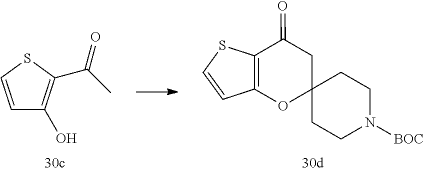

Compound 30c (156 g, 25.04 mmol) was dissolved in 20 mL of methanol and to the resulting solution was added pyrrolidine (2.1 mL, 25.16 mmol). The resulting reaction was allowed to stir for 15 minutes, then N-boc-4-piperidine ketone (4.92 g, 25.03 mmol) was added and the reaction was stirred for an additional 2.5 days. The reaction mixture was then concentrated in vacuo to provide a crude brown oil, which was dissolved in 120 mL of ethyl acetate, washed sequentially with a 1.0 M NaOH aqueous solution, H₂O, a 1.0 M HCl aqueous solution, H₂O, and brine. The organic solution was dried over Na₂SO₄, and concentrated in vacuo to provide compound 30d (4.41 g, ˜50%) as a brown oil.

### Step 4

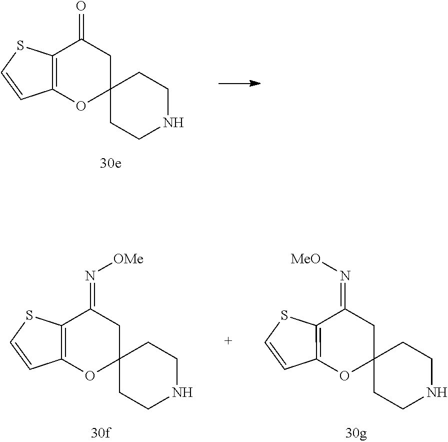

Compound 30e (0.51 g, 2.284 mmol, prepared from 30d using procedure analogous to that described in step 5 of example 3) was dissolved in 6 mL pyridine and to the resulting solution was added methoxylamine hydrochloride (1.90 g, 22.75 mmol). The resulting mixture was heated to 45° C. and allowed to stir at this temperature for 2 days, then concentrated in vacuo to provide an oily/solid residue. The residue was purified using preparative TLC (CH₂Cl₂-7 N NH₃ in MeOH=15:1, v/v, two elutions) to provide 70 mg of the E-oxime isomer 30f (12%) and 344 mg of the Z-oxime isomer 30g (60%).

### Step 5

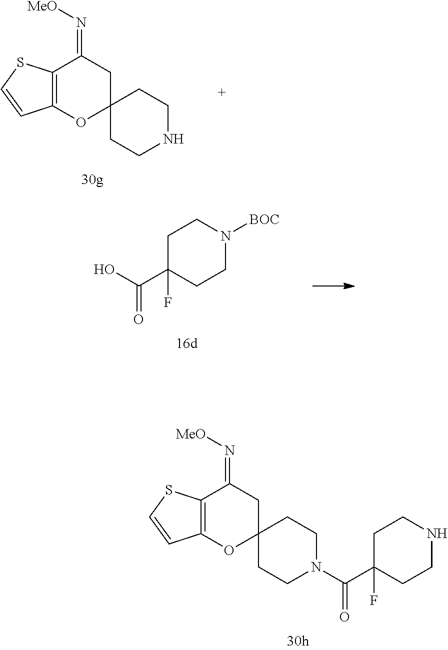

To a stirred solution of 16d (0.29 g, 1.173 mmol) in 10 mL of CH₂Cl₂ was added oxalyl chloride (0.12 mL, 1.38 mmol) followed by a catalytic amount of DMF. The mixture was stirred at room temperature for 1.5 h. Triethylamine (0.4 mL, 2.87 mmol) was added. A solution of 30g (0.34 g, 1.347 mmol) in 2 mL of CH₂Cl₂ was added. The reaction mixture was allowed to stir for 2.5 days, then was diluted with CH₂Cl₂ (50 mL), washed with a 1.0 M HCl aqueous solution and brine, dried over Na₂SO₄, and concentrated in vacuo to provide an oily solid residue. The residue was dissolved in 10 mL of CH₂Cl₂ and 2 mL of trifluoroacetic acid and allowed to stir for 5 hours, then concentrated in vacuo. The residue obtained was dissolved in 50 mL of CH₂Cl₂, washed with a saturated NaHCO₃ aqueous solution and brine.

The organic solution was dried, and concentrated to provide a crude oil, which was purified using preparative TLC (CH₂Cl₂-7N NH₃ in MeOH=25:1, v/v) to provide compound 30h (260 mg, 58%, MH⁺=382.2).

### Step 6

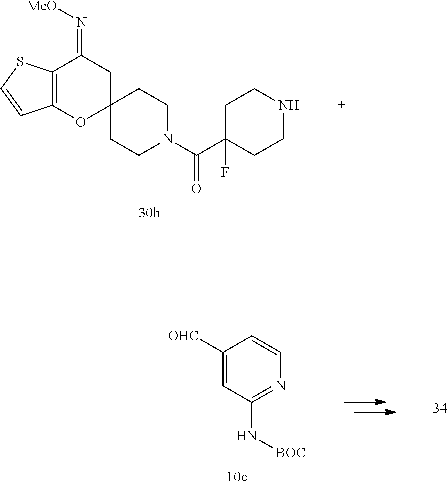

Compound 30h was converted to compound 34 (MH⁺=488) using the methods described in step 2 of example 10.

### Example 31

### Preparation of Compound 35

### Step 1

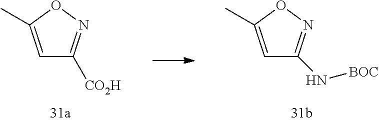

To a solution of compound 31a (3.0 g, 22.6 mmol) in tBuOH (50 mL) was added Et₃N (6.34 mL, 45.2 mmol) followed by DPPA (5.35 mL, 24.8 mmol). The resulting reaction was heated to reflux for 18 hours, then cooled to room temperature and concentrated in vacuo. The residue obtained was dissolved in dichloromethane, washed with 1N HCl, saturated NaHCO₃, brine, dried over Na₂SO₄ and concentrated in vacuo to provide a crude residue which was purified using flash column chromatography (EtOAc/Hexanes, 1:9 then 1:6) to provide compound 31b (2.0 g, 45%).

### Step 2

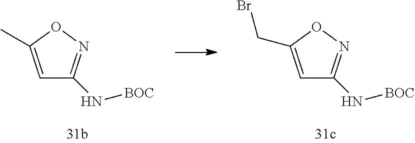

To a solution of compound 31b (1.55 g, 7.83 mmol) in CCl₄ (20 mL) was added NBS (5.58 g, 30.3 mmol) and benzoyl peroxide (283 mg, 1.17 mmol). The resulting reaction was heated to reflux for 48 hours, then cooled to room temperature, diluted with dichloromethane, washed with 10% Na₂S₂O₃, brine, dried over Na₂SO₄ and concentrated in vacuo. The residue obtained was purified using flash column chromatography (EtOAc/Hexanes, 1:9 then 1:6) to provide compound 31c (1.20 g, 56%).

### Step 3

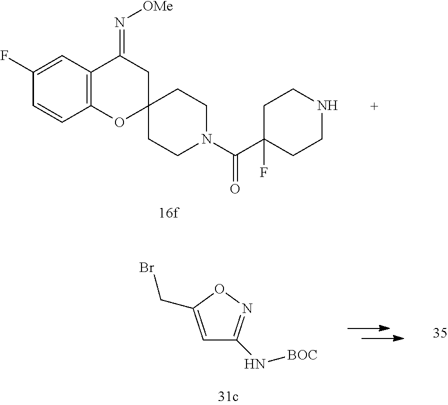

To a solution of compound 16f (see step 4 of example 16) (100 mg, 0.287 mmol) in DMF (2 mL) was added dry K₂CO₃ powder (200 mg) and compound 31c (40 mg, 0.143 mmol). The mixture was stirred at room temperature for 48 hours, then diluted with dichloromethane, filtered and concentrated in vacuo. The residue obtained was purified using preparative TLC (MeOH:dichloromethane/1:10) to provide a colorless oil, which was taken up in dichloromethane (5 mL) and TFA (5 mL) and allowed to stir for 1 hour, then concentrated in vacuo. The residue obtained was dissolved in dichloromethane and 1N NaOH and stirred for 10 minutes, then dichloromethane layer was washed with brine, dried over MgSO₄ and concentrated in vacuo. The resulting residue was purified using preparative TLC (MeOH:dichloromethane, 1:10) to provide compound 35 (11 mg, 20%). MH⁺=490

### Example 32

### Preparation of Compound 36

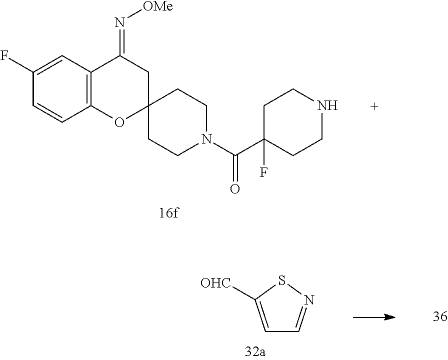

Compounds 16f and 32a (prepared as described in Kang, Y. K. et al., Bioorganic & Medicinal Chemistry Letters 2003, 13, 463-466) were reacted according to the method described in step 1 of example 18 to provide compound 36 (MH⁺=491).

### Example 33

### Preparation of Compound 37

### Step 1

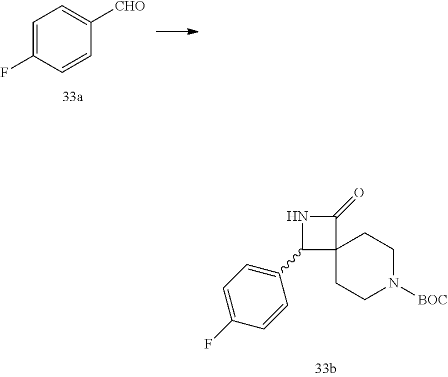

Compound 33a (5.32 g, 42.9 mmol) was taken up in 10 ml dry THF and the resulting solution was cooled to −20° C. under a nitrogen atmosphere. Lithium hexmethyldisilazane (1M THF, 43 ml, 43 mmol) was added in two portions over 20 minutes and the resulting reaction was allowed to stir at −20° C. for 1 hour, then the cold bath was removed and the reaction was allowed to warm slowly to 0° C. with stirring over an additional hour (Solution A).

A second flask was charged with diisopropyl amine (4.35 g, 43 mmol), 10 mL dry THF and cooled to −78° C. N-butyllithium (2.5 M in hexane, 17.3 mL, 43 mmol) was added via syringe pump over 10 minutes and the resulting reaction was stirred for 0.5 hours (Solution B).

A third flask was charged with n-boc nipecotic acid ethyl ester (9.3 g, 36 mmol) and 10 ml THF (solution C).

Solution C was then added dropwise to the solution B at −78° C. The resulting mixture was allowed to stir for 1.5 hours at −78° C., then Solution A was added dropwise at −78° C., and the resulting reaction was allowed to stir for 2 hours at −78° C., and then allowed to warm slowly to room temperature. The reaction was quenched with saturated aqueous ammonium chloride solution, then extracted into methylene chloride. The organic extract was concentrated in vacuo to provide a crude oil, and the product was crystallized from 1:1 hexane/ethylacetate to provide compound 33b as white solid (8.7 g, 73%). The mother liquor was subsequently chromatographed to provide an additional amound of compound 33b (1.2 g).

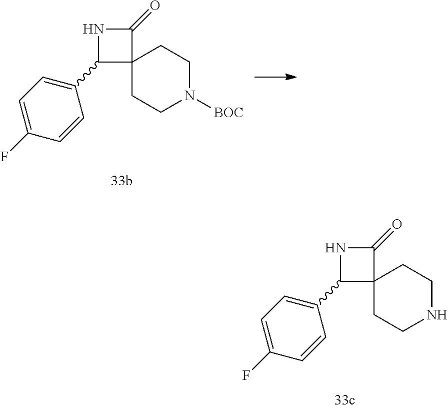

### Step 2

A flask was charged with 100 ml 1,4-dioxane and the compound 33b above (7.8 g, 23.3 mmol). A solution of hydrochloric acid (4N in dioxane, 40 ml) was added slowly and the reaction was allowed to stir for about 15 hours. The solution was concentrated to approximately half volume and stand an additional 24 hours. The product 33c was isolated by filtration as the mono-HCl salt (6.3 g, 100%).

### Step 3

Compound 33c was converted to compound 37 (MH⁺=452) using the methods described in steps 4 and 5 of example 3.

### Example 34

### Preparation of Compound 38

### Step 1

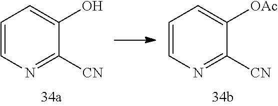

To a solution of 2-cyano-3-hydroxypyridine 34a (2.03 g; 16.9 mmol) in 40 mL of pyridine at 45° C. was added 1.92 mL (20.3 mmol) of acetic anhydride. The resulting reaction was allowed to stir for 24 hours at 45° C., then an additional 1.92 mL of acetic anhydride was added. After another 24 hours of stirring at 45° C. the reaction mixture was concentrated in vacuo and subjected to aqueous NaHCO₃ work-up—CH₂Cl₂ extraction to provide compound 34b as a yellow oil, which was used without further purification.

### Step 2

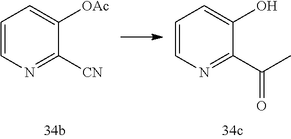

A solution of crude compound 34b in 30 mL of benzene was added to a 0° C. solution of MeLi (80 mL of 1.6M soln. in ether, 128 mmol) over 20 minutes. The mixture was then allowed to stir for 8 hours at 70° C. and cooled to room temperature. Excess MeLi was decomposed by the addition of saturated aqueous NH₄Cl (until gas evolution ceased) and further diluted with water. The aqueous phase was acidified to pH 6 using 1N HCl, then extracted with ether and CH₂Cl₂. The combined organic extracts were concentrated in vacuo, and the residue obtained was purified using flash column chromatography (5-10% acetone/CH₂Cl₂) to provide compound 34c (1.35 g; 9.85 mmol; 58% over two steps) as an off-white solid.

### Step 3

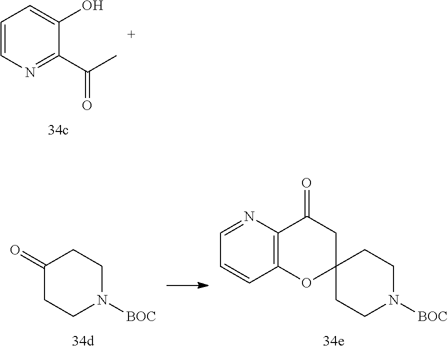

A mixture of compound 34c (477 mg; 3.48 mmol), compound 34d (693 mg; 3.48 mmol) and pyrrolidine (142 μL; 1.70 mmol) in 25 mL of methanol was stirred at reflux for 24 hours, then the methanol was removed in vacuo. The residue obtained was diluted with water, extracted with CH₂Cl₂, and the organic phase was dried over Na₂SO₄, filtered and concentrated in vacuo. The resulting residue was purified using flash chromatography (8-15% acetone/CH₂Cl₂) to provide compound 34e (340 mg; 1.07 mmol; 31%) as a white foam.

### Step 4

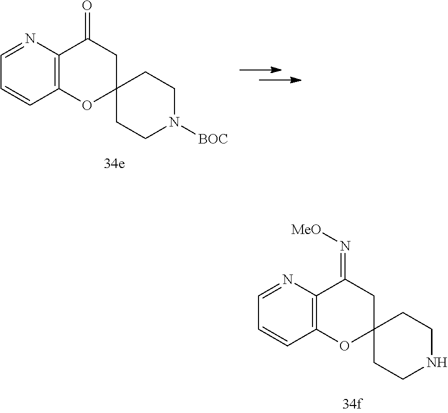

To a solution of compound 34e (330 mg; 1.03 mmol) in 8 mL of pyridine was added methoxylamine hydrochloride (585 mg; 7 mmol). The resulting reaction was stirred at 85° C. for about 15 hours, then concentrated in vacuo and the residue obtained was diluted with water and extracted with CH₂Cl₂. The organic phase was concentrated to provide 370 mg of crude product as a white foam. This crude material was stirred for about 15 hours in 20% TFA/CH₂Cl₂ at room temperature, then concentrated in vacuo, diluted with saturated aqueous NaHCO₃ solution and extracted with CH₂Cl₂. The organic phase was dried over Na₂SO₄, filtered and concentrated in vacuo to provide compound 34f (155 mg) as a yellow oil, which was used without further purification.

### Step 5

Compound 34f was converted to compound 38 using the method described of step 2 of example 8, followed by the method described in step 5 of example 3. MH⁺=483

### Example 35

### Preparation of Compound 39

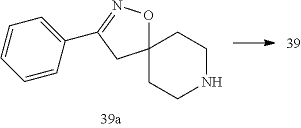

Compound 39 was prepared from compound 39a (prepared as described in WO 2006122770) using the method described in step 2 of example 8, followed by the method described in step 5 of example 3.

### Example 36

### Preparation of Compound 102

### Step 1

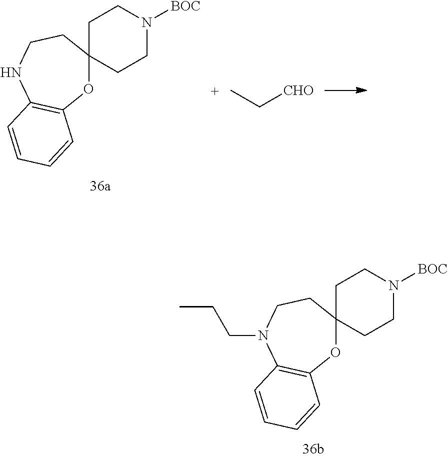

Compound 36a (prepared as described in Tetrahedron Letters 2004, 45, pp. 1051-1054) was converted into compound 36b using the reductive amination conditions (NaBH(OAc)₃, CH₂Cl₂) described in International Publication No. WO 02/032893.

### Step 2

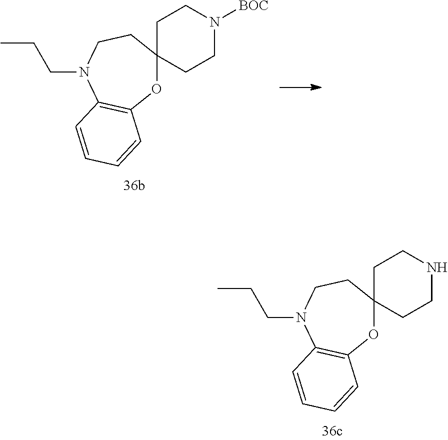

Compound 36b was converted to 36c using the method described in step 5 of example 3.

### Step 3

Compound 36c was converted to compound 102 using the methods described in steps 4 and 5 of example 3.

### Example 37

### H₃ Receptor Binding Assay

The source of the H₃ receptors in this experiment was guinea pig brain. Alternatively, the source of H₃ receptors was recombinant human receptor, expressed in HEK-293 (human embryonic kidney) cells.

The animals weighed 400-600 g. The brain tissue was homogenized with a solution of 50 mM Tris, pH 7.5. The final concentration of tissue in the homogenization buffer was 10% w/v. The homogenates were centrifuged at 1,000×g for 10 minutes. in order to remove clumps of tissue and debris. The resulting supernatants were then centrifuged at 50,000×g for 20 minutes. in order to sediment the membranes, which were next washed three times in homogenization buffer (50,000×g for 20 minutes, each). The membranes were frozen and stored at −70° C. until needed.

Compounds of the invention to be tested were dissolved in DMSO and then diluted into the binding buffer (50 mM Tris, pH 7.5) such that the final concentration was 2 μg/ml with 0.1% DMSO. Membranes were then added (400 μg of protein, 5 μg in the case of recombinant human receptor) to the reaction tubes. The reaction was started by the addition of 3 nM [³H]R-α-methyl histamine (8.8 Ci/mmol) or 3 nM [³H]N^{α}-methyl histamine (80 Ci/mmol) and continued under incubation at 30° C. for 30 minutes. Bound ligand was separated from unbound ligand by filtration, and the amount of radioactive ligand bound to the membranes was quantitated by liquid scintillation spectrometry. All incubations were performed in duplicate and the standard error was always less than 10%. Compounds that inhibited more than 70% of the specific binding of radioactive ligand to the receptor were serially diluted to determine a Kᵢ (nM).

Using this method it was shown that the compounds of the present invention demonstrate Kᵢ values of from about 2 nM to about 1000 nM at the recombinant human H₃ receptor and from about 8 nM to about 130 nM at the guinea pig brain H₃ receptor.

### Example 38

### In Vivo Effect of Compounds of the Invention on Glucose Levels in Diabetic Mice

Five-week-old male ICR mice (which can be purchased for example, from Taconic Farm, Germantown, N.Y.) are placed on a “western diet” containing 45% (kcal) fat from lard and 0.12% (w/w) cholesterol. After 3 weeks of feeding, the mice are injected once with low dose streptozocin (STZ, ip 75-100 mg/kg) to induce partial insulin deficiency. Two weeks after receiving the STZ injection, the animals that have developed type 2 diabetes and display hyperglycemia, insulin resistance, and glucose intolerance are placed in one of three groups: (1) a non-treated control group, (2) a group treated with rosiglitazone (5 mg/kg/day in diet); or (3) a group treated with a compound of the present invention (10/mg/kg in diet). The animals in groups (2) and (3) are treated daily at the designated dosages for total period of four weeks. The glucose levels in the three groups can then be compared to determine the effectiveness of the compounds of the invention in lowering glucose levels in the diabetic animals.

### Example 39

### In Vivo Effect of Compounds of the Invention on Glucose Levels in Diabetic Rats

Adult, diabetic, Goto-Kakizaki rats (14 weeks old) are tested for non-fasting glucose levels using a glucometer. Rats with glucose levels between 130 and 370 mg/dl are then randomized into treatment (N=10) and control (N=10) groups. Animals in the treatment group are administered a compound of the present invention in their food chow at a dose of 10 mg/kg/day. After one week of treatment, blood is collected via tail snip and the non-fasting glucose level is measured using a glucometer. The glucose levels of the animals in the treated group are compared to the glucose levels of the animals in the control group to determine the effectiveness of the test compound in lowering glucose levels in the diabetic animals.

### Example 40

### In Vivo Effect of Compounds of the Invention on Obese Mice

Five-week old mice (20-25 g; Jackson lab, Maine) were maintained in individual cages at 22° C. on a 12:12 hour light/dark cycle with lights on at 1100. Mice (n=12 per group) were balanced by body weight and food intake while on a standard laboratory chow (Teklad, formulation 2001) after an oral dosing with vehicle (20% hpbcd; 1 mL/kg). The following week, mice were switched from a chow diet into a high fat diet HF (Research Diets, New Brunswick, N.J., formulation #D12451, 4.7 kcal/g, comprised of 45% fat, 35% CHO, 20% protein). Daily oral gavage of vehicle or compound in vehicle occurred about the same time each day, approximately 1 hour before dark onset. Each day, HF pellets were pre-weighed and placed in the home cage immediately following daily oral gavage. Body weight and HF food intake were monitored daily for 4 days.

Using this method, it was demonstrated that selected illustrative compounds of the present invention, when administered at doses 1-30 mg/kg/day by oral gavage, significantly reduced body weights relative to control mice. Accordingly, the compounds of the present invention are useful for treating obesity.

### Uses of the Compounds of Formula (I)

The Compounds of Formula (I) are useful in human and veterinary medicine for treating or preventing a Condition in a patient. In accordance with the invention, the Compounds of Formula (I) can be administered to a patient in need of treatment or prevention of a Condition.

Accordingly, in one embodiment, the invention provides methods for treating a Condition in a patient comprising administering to the patient an effective amount of one or more compounds of Formula (I) or a pharmaceutically acceptable salt, solvate, ester or prodrug thereof. In addition, the present invention provides methods for treating or preventing Condition in a patient, comprising administering to the patient one or more Compounds of Formula (I) and an additional therapeutic agent that is not a Compound of Formula (I), wherein the amounts administered are together effective to treat or prevent the Condition.

In one embodiment, the compounds of the present invention can be ligands for the histamine H₃ receptor. In another embodiment, the compounds of the present invention can also be described as antagonists of the H₃ receptor, or as H₃ antagonists.

### Treating or Preventing Allergy

The Compounds of Formula (I) are useful for treating or preventing allergy in a patient.

Accordingly, in one embodiment, the present invention provides a method for treating allergy in a patient, comprising administering to the patient an effective amount of one or more Compounds of Formula (I).

Non-limiting examples of allergy treatable or preventable using the present methods include Type I hypersensitivity reactions, Type II hypersensitivity reactions, Type III hypersensitivity reactions, Type IV hypersensitivity reactions, food allergies, allergic lung disorders, allergic reaction to a venomous sting or bite; mold allergies, environmental-related allergies (such allergic rhinitis, grass allergies and pollen allergies), anaphlaxis and latex allergy.

In one embodiment, the allergy is an environmental-related allergy.

### Treating or Preventing Allergy-Induced Airway Response

The Compounds of Formula (I) are useful for treating or preventing allergy-induced airway response in a patient.

Accordingly, in one embodiment, the present invention provides a method for treating allergy-induced airway response in a patient, comprising administering to the patient an effective amount of one or more Compounds of Formula (I).

Non-limiting examples of allergy-induced airway response treatable or preventable using the present methods include upper airway responses.

In one embodiment, the allergy-induced airway response is an upper airway response.

### Treating or Preventing Congestion

The Compounds of Formula (I) are useful for treating or preventing congestion in a patient.

Accordingly, in one embodiment, the present invention provides a method for treating congestion in a patient, comprising administering to the patient an effective amount of one or more Compounds of Formula (I).

Non-limiting examples of congestion treatable or preventable using the present methods include nasal congestion and all types of rhinitis, including atrophic rhinitis, vasomotor rhinitis, gustatory rhinitis and drug induced rhinitis.

In one embodiment, the congestion is nasal congestion.

### Treating or Preventing a Neurological Disorder

The Compounds of Formula (I) are useful for treating or preventing a neurological disorder in a patient. The term “neurological disorder,” as used herein, refers to a disorder of any part of the central nervous system, including, but not limited to, the brain, nerves and spinal cord.

Accordingly, in one embodiment, the present invention provides a method for treating a neurological disorder in a patient, comprising administering to the patient an effective amount of one or more Compounds of Formula (I).

Non-limiting examples of neurological disorders treatable or preventable using the present methods include pain, hypotension, meningitis, a movement disorder (such as Parkinson's disease or Huntington's disease), delirium, dementia, Alzheimer's disease, a demyelinating disorder (such as multiple sclerosis or amyotrophic lateral sclerosis), aphasia, a peripheral nervous system disorder, a seizure disorder, a sleep disorder, a spinal cord disorder, stroke, a congnition deficit disorder (such as attention deficit hyperactivity disorder (ADHD)), hypo and hyperactivity of the central nervous system (such as agitation or depression) and schizophrenia.

In one embodiment, the neurological disorder is a sleep disorder.

In another embodiment, the neurological disorder is a movement disorder.

In another embodiment, the neurological disorder is Alzheimer's disease.

In yet another embodiment, the neurological disorder is schizophrenia.

In another embodiment, the neurological disorder is hypotension.

In one another embodiment, the neurological disorder is depression.

In another embodiment, the neurological disorder is a cognition deficit disorder.

In a further embodiment, the neurological disorder is ADHD, which can be present in an adult or a child.

In one embodiment, the sleep disorder is hypersomnia, somnolence or narcolepsy.

In another embodiment, the movement disorder is Parkinson's disease or Huntington's disease.

In one embodiment, the neurological disorder is pain.

Non-limiting examples of pain treatable or preventable using the present methods include acute pain, chronic pain, neuropathic pain, nociceptive pain, cutaneous pain, somatic pain, visceral pain, phantom limb pain, cancer pain (including breakthrough pain), pain caused by drug therapy (such as cancer chemotherapy), headache (including migraine, tension headache, cluster headache, pain caused by arthritis, pain caused by injury, toothache, or pain caused by a medical procedure (such as surgery, physical therapy or radiation therapy).

In one embodiment, the pain is neuropathic pain.

In another embodiment, the pain is cancer pain.

In another embodiment, the pain is headache.

### Treating or Preventing a Cardiovascular Disease

The Compounds of Formula (I) are useful for treating or preventing a cardiovascular disease in a patient.

Accordingly, in one embodiment, the present invention provides a method for treating a cardiovascular disease in a patient, comprising administering to the patient an effective amount of one or more Compounds of Formula (I).

Examples of cardiovascular diseases treatable or preventable using the present methods include, but are not limited to, an arrhythmia, an atrial fibrillation, a supraventricular tachycardia, arterial hypertension, arteriosclerosis, coronary artery disease, pulmonary artery disease, a cardiomyopathy, pericarditis, a peripheral artery disorder, a peripheral venous disorder, a peripheral lymphatic disorder, congestive heart failure, myocardial infarction, angina, a valvular disorder or stenosis.

In one embodiment, the cardiovascular disease is atherosclerosis.

In another embodiment, the cardiovascular disease is coronary artery disease.

### Treating or Preventing a Gastrointestinal Disorder

The Compounds of Formula (I) are useful for treating or preventing a gastrointestinal disorder in a patient.

Accordingly, in one embodiment, the present invention provides a method for treating a gastrointestinal disorder in a patient, comprising administering to the patient an effective amount of one or more Compounds of Formula (I).

Examples of gastrointestinal disorders treatable or preventable using the present methods include, but are not limited to, hyper or hypo motility of the GI tract, acidic secretion of the GI tract, an anorectal disorder, diarrhea, irritable bowel syndrome, dyspepsis, gastroesophageal reflux disease (GERD), diverticulitis, gastritis, peptic ulcer disease, gastroenteritis, inflammatory bowel disease, a malabsorption syndrome or pancreatitis.

In one embodiment, the gastrointestinal disorder is GERD.

In another embodiment, the gastrointestinal disorder is hyper or hypo motility of the GI tract.

### Treating or Preventing an Inflammatory Disease

The Compounds of Formula (I) are useful for treating or preventing an inflammatory disease in a patient.

Accordingly, in one embodiment, the present invention provides a method for treating an inflammatory disease in a patient, comprising administering to the patient an effective amount of one or more Compounds of Formula (I).

### Treating or Preventing Non-Alcoholic Fatty Liver Disease

The Compounds of Formula (I) are useful for treating or preventing non-alcoholic fatty liver disease in a patient.

Accordingly, in one embodiment, the present invention provides a method for treating non-alcoholic fatty liver disease in a patient, comprising administering to the patient an effective amount of one or more Compounds of Formula (I).

### Treating or Preventing a Metabolic Disorder

The Compounds of Formula (I) can be useful for treating a metabolic disorder. Accordingly, in one embodiment, the invention provides methods for treating a metabolic disorder in a patient, wherein the method comprises administering to the patient an effective amount of one or more Compounds of Formula (I), or a pharmaceutically acceptable salt, solvate, ester or prodrug thereof.

Examples of metabolic disorders treatable include, but are not limited to, metabolic syndrome (also known as “Syndrome X”), impaired glucose tolerance, impaired fasting glucose, dyslipidemia, hypercholesterolemia, hyperlipidemia, hypertriglyceridemia, low HDL levels, hypertension, phenylketonuria, post-prandial lipidemia, a glycogen-storage disease, Gaucher's Disease, Tay-Sachs Disease, Niemann-Pick Disease, ketosis and acidosis.

In one embodiment, the metabolic disorder is hypercholesterolemia.

In another embodiment, the metabolic disorder is hyperlipidemia.

In another embodiment, the metabolic disorder is hypertriglyceridemia.

In still another embodiment, the metabolic disorder is metabolic syndrome.

In a further embodiment, the metabolic disorder is low HDL levels.

In another embodiment, the metabolic disorder is dyslipidemia.

### Treating or Preventing Obesity and Obesity-Related Disorders

The Compounds of Formula (I) can be useful for treating obesity or an obesity-related disorder. Accordingly, in one embodiment, the invention provides methods for treating obesity or an obesity-related disorder in a patient, wherein the method comprises administering to the patient an effective amount of one or more Compounds of Formula (I), or a pharmaceutically acceptable salt, solvate, ester or prodrug thereof.

### Methods For Treating or Preventing Diabetes

The Compounds of Formula (I) are useful for treating or preventing diabetes in a patient. Accordingly, in one embodiment, the present invention provides a method for treating diabetes in a patient, comprising administering to the patient an effective amount of one or more Compounds of Formula (I).

Examples of diabetes treatable or preventable using the Compounds of Formula (I) include, but are not limited to, type I diabetes (insulin-dependent diabetes mellitus), type II diabetes (non-insulin dependent diabetes mellitus), gestational diabetes, diabetes caused by administration of anti-psychotic agents, diabetes caused by administration of anti-depressant agents, diabetes caused by administration of steroid drugs, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), type A insulin resistance syndrome, type B insulin resistance syndrome, lipatrophic diabetes, diabetes induced by n-cell toxins, and diabetes induced by drug therapy (such as diabetes induced by antipsychotic agents).

In one embodiment, the diabetes is type I diabetes.

In another embodiment, the diabetes is type II diabetes.

In another embodiment, the diabetes is gestational diabetes.

### Methods For Treating or Preventing a Diabetic Complication

The Compounds of Formula (I) are useful for treating or preventing a diabetic complication in a patient. Accordingly, in one embodiment, the present invention provides a method for treating a diabetic complication in a patient, comprising administering to the patient an effective amount of one or more Compounds of Formula (I).

Examples of diabetic complications treatable or preventable using the Compounds of Formula (I) include, but are not limited to, diabetic cataract, glaucoma, retinopathy, aneuropathy (such as diabetic neuropathy, polyneuropathy, mononeuropathy, autonomic neuropathy, microaluminuria and progressive diabetic neuropathyl), nephropathy, diabetic pain, gangrene of the feet, immune-complex vasculitis, systemic lupsus erythematosus (SLE), atherosclerotic coronary arterial disease, peripheral arterial disease, nonketotic hyperglycemic-hyperosmolar coma, foot ulcers, joint problems, a skin or mucous membrane complication (such as an infection, a shin spot, a candidal infection or necrobiosis lipoidica diabeticorumobesity), hyperlipidemia, hypertension, syndrome of insulin resistance, coronary artery disease, a fungal infection, a bacterial infection, and cardiomyopathy.

In one embodiment, the diabetic complication is neuropathy.

In another embodiment, the diabetic complication is retinopathy.

In another embodiment, the diabetic complication is nephropathy.

### Methods For Treating or Preventing Impaired Glucose Tolerance

The Compounds of Formula (I) are useful for treating or preventing impaired glucose tolerance in a patient.

Accordingly, in one embodiment, the present invention provides a method for treating impaired glucose tolerance in a patient, comprising administering to the patient an effective amount of one or more Compounds of Formula (I).

### Methods For Treating or Preventing Impaired Fasting Glucose

The Compounds of Formula (I) are useful for treating or preventing impaired fasting glucose in a patient.

Accordingly, in one embodiment, the present invention provides a method for treating impaired fasting glucose in a patient, comprising administering to the patient an effective amount of one or more Compounds of Formula (I).

### Combination Therapy

Accordingly, in one embodiment, the present invention provides methods for treating a Condition in a patient, the method comprising administering to the patient one or more Compounds of Formula (I), or a pharmaceutically acceptable salt or solvate thereof and at least one additional therapeutic agent that is not a Compound of Formula (I), wherein the amounts administered are together effective to treat or prevent a Condition.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts).

In one embodiment, the one or more Compounds of Formula (I) is administered during at time when the additional therapeutic agent(s) exert their prophylactic or therapeutic effect, or vice versa.

In another embodiment, the one or more Compounds of Formula (I) and the additional therapeutic agent(s) are administered in doses commonly employed when such agents are used as monotherapy for treating a Condition.

In another embodiment, the one or more Compounds of Formula (I) and the additional therapeutic agent(s) are administered in doses lower than the doses commonly employed when such agents are used as monotherapy for treating a Condition.

In still another embodiment, the one or more Compounds of Formula (I) and the additional therapeutic agent(s) act synergistically and are administered in doses lower than the doses commonly employed when such agents are used as monotherapy for treating a Condition.

In one embodiment, the one or more Compounds of Formula (I) and the additional therapeutic agent(s) are present in the same composition. In one embodiment, this composition is suitable for oral administration. In another embodiment, this composition is suitable for intravenous administration.

The one or more Compounds of Formula (I) and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

In one embodiment, the administration of one or more Compounds of Formula (I) and the additional therapeutic agent(s) may inhibit the resistance of a Condition to these agents.

In one embodiment, when the patient is treated for diabetes, a diabetic complication, impaired glucose tolerance or impaired fasting glucose, the other therapeutic is an antidiabetic agent which is not a Compound of Formula (I). In another embodiment, when the patient is treated for pain, the other therapeutic agent is an analgesic agent which is not a Compound of Formula (I).

In another embodiment, the other therapeutic agent is an agent useful for reducing any potential side effect of a Compound of Formula (I). Such potential side effects include, but are not limited to, nausea, vomiting, headache, fever, lethargy, muscle aches, diarrhea, general pain, and pain at an injection site.

In one embodiment, the other therapeutic agent is used at its known therapeutically effective dose. In another embodiment, the other therapeutic agent is used at its normally prescribed dosage. In another embodiment, the other therapeutic agent is used at less than its normally prescribed dosage or its known therapeutically effective dose.

Examples of antidiabetic agents useful in the present methods for treating diabetes or a diabetic complication include a sulfonylurea; an insulin sensitizer (such as a PPAR agonist, a DPP-IV inhibitor, a PTP-1B inhibitor and a glucokinase activator); a glucosidase inhibitor; an insulin secretagogue; a hepatic glucose output lowering agent; an anti-obesity agent; an antihypertensive agent; a meglitinide; an agent that slows or blocks the breakdown of starches and sugars in vivo; an histamine H₃ receptor antagonist; an antihypertensive agent, a sodium glucose uptake transporter 2 (SGLT-2) inhibitor; a peptide that increases insulin production; and insulin or any insulin-containing composition.

In one embodiment, the antidiabetic agent is an insulin sensitizer or a sulfonylurea.

Non-limiting examples of sulfonylureas include glipizide, tolbutamide, glyburide, glimepiride, chlorpropamide, acetohexamide, gliamilide, gliclazide, glibenclamide and tolazamide.

Non-limiting examples of insulin sensitizers include PPAR activators, such as troglitazone, rosiglitazone, pioglitazone and englitazone; biguanidines such as metformin and phenformin; DPP-IV inhibitors; PTP-1B inhibitors; and α-glucokinase activators, such as miglitol, acarbose, and voglibose.

Non-limiting examples of DPP-IV inhibitors useful in the present methods include sitagliptin, saxagliptin (Januvia™, Merck), denagliptin, vildagliptin (Galvus™, Novartis), alogliptin, alogliptin benzoate, ABT-279 and ABT-341 (Abbott), ALS-2-0426 (Alantol), ARI-2243 (Arisaph), BI-A and BI-B (Boehringer Ingelheim), SYR-322 (Takeda), MP-513 (Mitsubishi), DP-893 (Pfizer), RO-0730699 (Roche) or a combination of sitagliptin/metformin HCl (Janumet™, Merck).

Non-limiting examples of SGLT-2 inhibitors useful in the present methods include dapagliflozin and sergliflozin, AVE2268 (Sanofi-Aventis) and T-1095 (Tanabe Seiyaku).

Non-limiting examples of hepatic glucose output lowering agents include Glucophage and Glucophage XR.

Non-limiting examples of histamine H₃ receptor antagonist agents include the following compound:

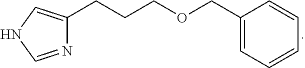

Non-limiting examples of insulin secretagogues include sulfonylurea and non-sulfonylurea drugs such as GLP-1, a GLP-1 mimetic, exendin, GIP, secretin, glipizide, chlorpropamide, nateglinide, meglitinide, glibenclamide, repaglinide and glimepiride.

Non-limiting examples of GLP-1 mimetics useful in the present methods include Byetta-Exanatide, Liraglutinide, CJC-1131 (ConjuChem, Exanatide-LAR (Amylin), BIM-51077 (Ipsen/LaRoche), ZP-10 (Zealand Pharmaceuticals), and compounds disclosed in International Publication No. WO 00/07617.

The term “insulin” as used herein, includes all formualtions of insulin, including long acting and short acting forms of insulin.

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference,

In one embodiment, the antidiabetic agent is anti-obesity agent.

Non-limiting examples of anti-obesity agents useful in the present methods for treating diabetes include a 5-HT2C agonist, such as lorcaserin; a neuropeptide Y antagonist; an MCR4 agonist; an MCH receptor antagonist; a protein hormone, such as leptin or adiponectin; an AMP kinase activator; and a lipase inhibitor, such as orlistat. Appetite suppressants are not considered to be within the scope of the anti-obesity agents useful in the present methods.

Non-limiting examples of antihypertensive agents useful in the present methods for treating diabetes include β-blockers and calcium channel blockers (for example diltiazem, verapamil, nifedipine, amlopidine, and mybefradil), ACE inhibitors (for example captopril, lisinopril, enalapril, spirapril, ceranopril, zefenopril, fosinopril, cilazopril, and quinapril), AT-1 receptor antagonists (for example losartan, irbesartan, and valsartan), renin inhibitors and endothelin receptor antagonists (for example sitaxsentan).

Non-limiting examples of meglitinides useful in the present methods for treating diabetes include repaglinide and nateglinide.

Non-limiting examples of insulin sensitizing agents include biguanides, such as metformin, metformin hydrochloride (such as GLUCOPHAGE® from Bristol-Myers Squibb), metformin hydrochloride with glyburide (such as GLUCOVANCE™ from Bristol-Myers Squibb) and buformin; glitazones; and thiazolidinediones, such as rosiglitazone, rosiglitazone maleate (AVANDIA™ from GlaxoSmithKline), pioglitazone, pioglitazone hydrochloride (ACTOS™, from Takeda) ciglitazone and MCC-555 (Mitstubishi Chemical Co.)

In one embodiment, the insulin sensitizer is a thiazolidinedione.

In another embodiment, the insulin sensitizer is a biguanide.

In another embodiment, the insulin sensitizer is a DPP-IV inhibitor.

In a further embodiment, the antidiabetic agent is a SGLT-2 inhibitor.

Non-limiting examples of antidiabetic agents that slow or block the breakdown of starches and sugars and are suitable for use in the compositions and methods of the present invention include alpha-glucosidase inhibitors and certain peptides for increasing insulin production. Alpha-glucosidase inhibitors help the body to lower blood sugar by delaying the digestion of ingested carbohydrates, thereby resulting in a smaller rise in blood glucose concentration following meals. Non-limiting examples of suitable alpha-glucosidase inhibitors include acarbose; miglitol; camiglibose; certain polyamines as disclosed in WO 01/47528 (incorporated herein by reference); voglibose. Non-limiting examples of suitable peptides for increasing insulin production including amlintide (CAS Reg. No. 122384-88-7 from Amylin; pramlintide, exendin, certain compounds having Glucagon-like peptide-1 (GLP-1) agonistic activity as disclosed in WO 00/07617 (incorporated herein by reference).

Non-limiting examples of orally administrable insulin and insulin containing compositions include AL-401 from Autoimmune, and the compositions disclosed in U.S. Pat. Nos. 4,579,730; 4,849,405; 4,963,526; 5,642,868; 5,763,396; 5,824,638; 5,843,866; 6,153,632; 6,191,105; and International Publication No. WO 85/05029, each of which is incorporated herein by reference.

Non-limiting examples of other analgesic agents useful in the present methods for treating pain include acetaminophen, an NSAID, an opiate or a tricyclic antidepressant.

In one embodiment, the other analgesic agent is acetaminophen or an NSAID.

In another embodiment, the other analgesic agent is an opiate.

In another embodiment, the other analgesic agent is a tricyclic antidepressant.

Non-limiting examples of NSAIDS useful in the present methods for treating pain include a salicylate, such as aspirin, amoxiprin, benorilate or diflunisal; an arylalkanoic acid, such as diclofenac, etodolac, indometacin, ketorolac, nabumetone, sulindac or tolmetin; a 2-arylpropionic acid (a “profen”), such as ibuprofen, carprofen, fenoprofen, flurbiprofen, loxoprofen, naproxen, tiaprofenic acid or suprofen; a fenamic acid, such as mefenamic acid or meclofenamic acid; a pyrazolidine derivative, such as phenylbutazone, azapropazone, metamizole or oxyphenbutazone; a coxib, such as celecoxib, etoricoxib, lumiracoxib or parecoxib; an oxicam, such as piroxicam, lornoxicam, meloxicam or tenoxicam; or a sulfonanilide, such as nimesulide.

Non-limiting examples of opiates useful in the present methods for treating pain include an anilidopiperidine, a phenylpiperidine, a diphenylpropylamine derivative, a benzomorphane derivative, an oripavine derivative and a morphinane derivative. Additional illustrative examples of opiates include morphine, diamorphine, heroin, buprenorphine, dipipanone, pethidine, dextromoramide, alfentanil, fentanyl, remifentanil, methadone, codeine, dihydrocodeine, tramadol, pentazocine, vicodin, oxycodone, hydrocodone, percocet, percodan, norco, dilaudid, darvocet or lorcet.

Non-limiting examples of tricyclic antidepressants useful in the present methods for treating pain include amitryptyline, carbamazepine, gabapentin or pregabalin.

The Compounds of Formula (I) can be combined with an H₁ receptor antagonist (i.e., the Compounds of Formula (I) can be combined with an H₁ receptor antagonist in a pharmaceutical composition, or the Compounds of Formula (I) can be administered with one or more H₁ receptor antagonists).

Numerous chemical substances are known to have histamine H₁ receptor antagonist activity and can therefore be used in the methods of this invention. Many H₁ receptor antagonists useful in the methods of this invention can be classified as ethanolamines, ethylenediamines, alkylamines, phenothiazines or piperidines. Representative H₁ receptor antagonists include, without limitation: astemizole, azatadine, azelastine, acrivastine, brompheniramine, cetirizine, chlorpheniramine, clemastine, cyclizine, carebastine, cyproheptadine, carbinoxamine, descarboethoxyloratadine, diphenhydramine, doxylamine, dimethindene, ebastine, epinastine, efletirizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine and triprolidine. Other compounds can readily be evaluated to determine activity at H₁ receptors by known methods, including specific blockade of the contractile response to histamine of isolated guinea pig ileum. See for example, WO98/06394 published Feb. 19, 1998.

Those skilled in the art will appreciate that the H₁ receptor antagonist is used at its known therapeutically effective dose, or the H₁ receptor antagonist is used at its normally prescribed dosage.

Preferably, said H₁ receptor antagonist is selected from: astemizole, azatadine, azelastine, acrivastine, brompheniramine, cetirizine, chlorpheniramine, clemastine, cyclizine, carebastine, cyproheptadine, carbinoxamine, descarboethoxyloratadine, diphenhydramine, doxylamine, dimethindene, ebastine, epinastine, efletirizine, fexofenadine, hydroxyzine, ketotifen, loratadine, levocabastine, meclizine, mizolastine, mequitazine, mianserin, noberastine, norastemizole, picumast, pyrilamine, promethazine, terfenadine, tripelennamine, temelastine, trimeprazine or triprolidine.

More preferably, said H₁ receptor antagonist is selected from: astemizole, azatadine, azelastine, brompheniramine, cetirizine, chlorpheniramine, clemastine, carebastine, descarboethoxyloratadine, diphenhydramine, doxylamine, ebastine, fexofenadine, loratadine, levocabastine, mizolastine, norastemizole, or terfenadine.

Most preferably, said H₁ receptor antagonist is selected from: azatadine, brompheniramine, cetirizine, chlorpheniramine, carebastine, descarboethoxy-loratadine, diphenhydramine, ebastine, fexofenadine, loratadine, or norastemizole.

Even more preferably, said H₁ antagonist is selected from loratadine, descarboethoxyloratadine, fexofenadine or cetirizine. Still even more preferably, said H₁ antagonist is loratadine or descarboethoxyloratadine.

In one preferred embodiment, said H₁ receptor antagonist is loratadine.

In another preferred embodiment, said H₁ receptor antagonist is descarboethoxyloratadine.

In still another preferred embodiment, said H₁ receptor antagonist is fexofenadine.

In yet another preferred embodiment, said H₁ receptor antagonist is cetirizine.

Preferably, in the above methods, allergy-induced airway responses are treated.

Also, preferably, in the above methods, allergy is treated.

Also, preferably, in the above methods, nasal congestion is treated.

In the methods of this invention wherein a combination of an H₃ antagonist of this invention (compound of formula I) is administered with a H₁ antagonist, the antagonists can be administered simultaneously or sequentially (first one and then the other over a period of time). In general, when the antagonists are administered sequentially, the H₃ antagonist of this invention (compound of formula I) is administered first.

The doses and dosage regimen of the other agents used in the combination therapies of the present invention for the treatment or prevention of a Condition can be determined by the attending clinician, taking into consideration the approved doses and dosage regimen in the package insert; the age, sex and general health of the patient; and the type and severity of the viral infection or related disease or disorder. When administered in combination, the Compound(s) of Formula (I) and the other agent(s) for treating diseases or conditions listed above can be administered simultaneously or sequentially. This is particularly useful when the components of the combination are given on different dosing schedules, e.g., one component is administered once daily and another every six hours, or when the preferred pharmaceutical compositions are different, e.g. one is a tablet and one is a capsule. A kit comprising the separate dosage forms is therefore advantageous.

Generally, a total daily dosage of the one or more Compounds of Formula (I) and the additional therapeutic agent(s) can, when administered as combination therapy, range from about 0.1 to about 2000 mg per day, although variations will necessarily occur depending on the target of the therapy, the patient and the route of administration. In one embodiment, the dosage is from about 0.2 to about 100 mg/day, administered in a single dose or in 2-4 divided doses. In another embodiment, the dosage is from about 1 to about 500 mg/day, administered in a single dose or in 2-4 divided doses. In another embodiment, the dosage is from about 1 to about 200 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 g/day, administered in a single dose or in 2-4 divided doses. In a further embodiment, the dosage is from about 1 to about 20 mg/day, administered in a single dose or in 2-4 divided doses.

### Compositions and Administration

In one embodiment, the invention provides compositions comprising an effective amount of one or more Compounds of Formula (I) or a pharmaceutically acceptable salt, solvate, ester or prodrug thereof, and a pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

In one embodiment, the Compound of Formula (I) is administered orally.

In another embodiment, the Compound of Formula (I) is administered parenterally.

In another embodiment, the Compound of Formula (I) is administered intravenously.

In one embodiment, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation is from about 0.1 to about 2000 mg. Variations will necessarily occur depending on the target of the therapy, the patient and the route of administration. In one embodiment, the unit dose dosage is from about 0.2 to about 1000 mg. In another embodiment, the unit dose dosage is from about 1 to about 500 mg. In another embodiment, the unit dose dosage is from about 1 to about 100 mg/day. In still another embodiment, the unit dose dosage is from about 1 to about 50 mg. In yet another embodiment, the unit dose dosage is from about 1 to about 10 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 300 mg/day, preferably 1 mg/day to 75 mg/day, in two to four divided doses.

When the invention comprises a combination of at least one Compound of Formula (I) and an additional therapeutic agent, the two active components may be co-administered simultaneously or sequentially, or a single pharmaceutical composition comprising at least one Compound of Formula (I) and an additional therapeutic agent in a pharmaceutically acceptable carrier can be administered. The components of the combination can be administered individually or together in any conventional dosage form such as capsule, tablet, powder, cachet, suspension, solution, suppository, nasal spray, etc. The dosage of the additional therapeutic agent can be determined from published material, and may range from about 1 to about 1000 mg per dose. In one embodiment, when used in combination, the dosage levels of the individual components are lower than the recommended individual dosages because of the advantageous effect of the combination.

In one embodiment, the components of a combination therapy regime are to be administered simultaneously, they can be administered in a single composition with a pharmaceutically acceptable carrier.

In another embodiment, when the components of a combination therapy regime are to be administered separately or sequentially, they can be administered in separate compositions, each containing a pharmaceutically acceptable carrier.

The components of the combination therapy can be administered individually or together in any conventional dosage form such as capsule, tablet, powder, cachet, suspension, solution, suppository, nasal spray, etc.

### Kits

In one aspect, the present invention provides a kit comprising a effective amount of one or more Compounds of Formula (I), or a pharmaceutically acceptable salt or solvate of the compound and a pharmaceutically acceptable carrier, vehicle or diluent.

In another aspect the present invention provides a kit comprising an amount of one or more Compounds of Formula (I), or a pharmaceutically acceptable salt or solvate of the compound and an amount of at least one additional therapeutic agent listed above, wherein the combined amounts are effective for treating or preventing a Condition in a patient.

When the components of a combination therapy regime are to are to be administered in more than one composition, they can be provided in a kit comprising in a single package, one container comprising a Compound of Formula (I) in pharmaceutically acceptable carrier, and one or more separate containers, each comprising one or more additional therapeutic agents in a pharmaceutically acceptable carrier, with the active components of each composition being present in amounts such that the combination is therapeutically effective.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1-52. (canceled)

53. A compound of the formula I: 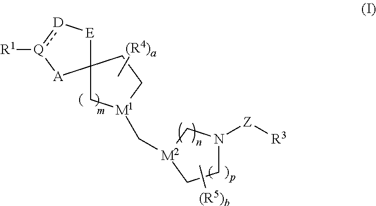
wherein the dotted line represents an optional and additional bond, and
wherein:
A is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)—, —C(═NH)N(R⁸)— or —N(R⁸)— or —C(R²¹)═N—;
D is —C(R²)— or —N— when the optional and additional bond is present, and D is —C(R²)₂—or —N(R²)— when the optional and additional bond is absent, such that when D is N and optional and additional bond is present, E is either —OC(R²¹)— or —O—;
E is a bond, —CH(R²¹)CH(R⁶)—, —CH(R²¹)C(O)—, —CH(R²¹)C(═N—OR⁹)—, —CH(R²¹)—, —C(O)CH(R⁶)—, —C(O)—, —C(═N—OR⁹)CH(R⁶)—, —C(═N—OR⁹)—, —C(═NH)N(R⁸)— —OCH(R²¹)— or —O— when D is —N—; and E is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —N(R⁸)CH(R⁶)CH(R²¹)—, —N(R⁸)C(O)CH(R²¹)—, —N(R⁸)C(═N—OR⁹)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)— or —N(R⁸)— when D is other than —N—;
M¹ is —CH— or —N—;
M² is —CH—, —CF— or —N—;
Q is —C— when the optional and additional bond is present, and Q is —CH— when the optional and additional bond is absent;
Y is alkylene, -alkylene-C(O)—, —C(O)-alkylene-, —C(O)—, —C(S)—, —SO₂— or —O—;
Z is a bond, alkylene, alkenylene or -(alkylene)ᵥ-cycloalkylene-(alkylene)ᵥ-;
R¹ is H, aryl, -alkylene-aryl, heteroaryl, -alkylene-heteroaryl, alkyl, cycloalkyl or heterocycloalkyl, any of which can be optionally substituted with up to 4 substituents, which can be the same or different, and are selected from halo, —OH, alkyl, haloalkyl, —O-alkyl, —O-haloalkyl, —NO₂, —C(O)₂R¹², —N(R¹²)₂, —C(O)N(R¹²)₂, —NHC(O)R¹², —NHSO₂R¹², —SO₂N(R¹²)₂ and —CN, or R¹ and R², together with Q and D, combine to form an aryl, hetroaryl, cycloalkyl or heterocycloalkyl ring, any of which can be optionally substituted with up to 4 substituents, which can be the same or different, and are selected from halo, —OH, alkyl, haloalkyl, —O-alkyl, —O-haloalkyl, —NO₂, —CO₂R¹², —N(R¹²)₂, —C(O)N(R¹²)₂, —NHC(O)R¹², —NHSO₂R¹², —SO₂N(R¹²)₂ and —CN;
each occurrence of R² is independently H, aryl, -alkylene-aryl, heteroaryl, -alkylene-heteroaryl, alkyl, cycloalkyl or heterocycloalkyl, any of which can be optionally substituted with up to 4 substituents, which can be the same or different, and are selected from halo, —OH, alkyl, haloalkyl, —O-alkyl, —O-haloalkyl, —NO₂, —C(O)₂R¹², —N(R¹²)₂, —C(O)N(R¹²)₂, —NHC(O)R¹², —NHSO₂R¹², —SO₂N(R¹²)₂ and —CN;
R³ is H, alkyl, R²²-aryl, R²²-cycloalkyl, R²²-heterocycloalkyl or R²²-heteroaryl;
R⁴ and R⁵ are each independently halo, alkyl, —OH, —O-alkyl, haloalkyl or —CN;
each occurrence of R⁶ is independently H, halo, alkyl, haloalkyl, aryl, heteroaryl, cycloalkyl, heterocycloalkyl, —OH, —N(R¹³)₂, —NHC(O)R¹⁴, —NHC(O)₂R¹⁴, —NHC(O)NR¹⁴ or —NHSO₂R¹⁴;
R⁸ is H, alkyl, haloalkyl, aryl, cycloalkyl, heterocycloalkyl, heteroaryl, -alkylene-aryl, -alkylene-heteroaryl, —C(O)₂R¹⁴, —C(O)NR¹⁴ or —S(O)₂R¹⁴, wherein an aryl group can be optionally substituted with one or more alkyl groups, which can be the same or different;
R⁹ is H, alkyl, haloalkyl, aryl or heteroaryl;
each occurrence of R¹² is independently H, alkyl, aryl or heteroaryl;
each occurrence of R¹³ is hydrogen or alkyl;
each occurrence of R¹⁴ is independently alkyl, haloalkyl, aryl or heteroaryl;
each occurrence of R²¹ is independently H, alkyl, haloalkyl, aryl, heteroaryl, cycloalkyl or heterocycloalkyl;
each occurrence of R²² represents from 1 to 4 substituents, each independently selected from H, alkyl, haloalkyl, aryl, heteroaryl, cycloalkyl and heterocycloalkyl, wherein an aryl, heteroaryl, cycloalkyl or heterocycloalkyl group can be optionally and independently substituted with from 1 to 4 groups, each independently selected from alkyl, halo, —CN, —NO₂, alkyl, —N(R²¹)₂, —C(O)OR²¹, —C(O)N(R²¹)₂, —NHC(O)R²¹, —S(O)_{q}R²¹ or —OR²¹;
R²⁵ is selected from the group consisting of H and alkyl;
a is 0, 1 or 2;
b is 0, 1 or 2;
m is an integer ranging from 1 to 3;
n is 1 or 2, such that when M² is N, then n is 2;
p is 1 or 2;
each occurrence of q is 0, 1 or 2; and
each occurrence of v is 0 or 1;
or a pharmaceutically acceptable salt thereof.

54. The compound of claim 53 of the formula Ia: 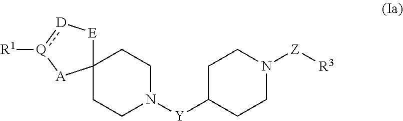
wherein:
A is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)—, —C(═NH)N(R⁸)— or —N(R⁸)— or —C(R²¹)═N—;
D is —C(R²)— or —N— when the optional and additional bond is present, and D is —C(R²)₂— or —N(R²)— when the optional and additional bond is absent, such that when D is N and optional and additional bond is present, E is either —OC(R²¹)— or —O—;
E is a bond, —CH(R²¹)CH(R⁶)—, —CH(R²¹)C(O)—, —CH(R²¹)C(═N—OR⁹)—, —CH(R²¹)—, —C(O)CH(R⁶)—, —C(O)—, —C(═N—OR⁹)CH(R⁶)—, —C(═N—OR⁹)—, —C(═NH)N(R⁸)— —OCH(R²¹)— or —O— when D is —N—; and E is a single bond, —CH(R⁶)CH(R⁶)—, —C(O)CH(R⁶)—, —C(═N—OR⁹)CH(R⁶)—, —CH(R⁶)C(O)—, —CH(R⁶)C(═N—OR⁹)—, —CH(R⁶)—, —C(O)—, —C(═N—OR⁹)—, —OCH(R²¹)—, —CH(R²¹)O—, —O—, —N(R⁸)CH(R²¹)—, —N(R⁸)CH(R⁶)CH(R²¹)—, —N(R⁸)C(O)CH(R²¹)—, —N(R⁸)C(═N—OR⁹)CH(R²¹)—, —CH(R²¹)N(R⁸)—, —N(R⁸)C(O)—, —C(O)N(R⁸)—, —N(R⁸)C(═N—OR⁹)—, —C(═N—OR⁹)N(R⁸)— or —N(R⁸)— when D is other than —N—;
Q is —C— when the optional and additional bond is present, and Q is —CH— when the optional and additional bond is absent;
Y is alkylene or —C(O)—;
Z is a bond, alkylene or alkenylene;
R¹ and R² are each independently H, aryl, -alkylene-aryl, heteroaryl, -alkylene-heteroaryl, alkyl, cycloalkyl or heterocycloalkyl, any of which can be optionally substituted with up to 4 substituents, which can be the same or different, and are selected from halo, —OH, alkyl, haloalkyl, —O-alkyl, —O-haloalkyl, —NO₂, —CO₂R¹², —N(R¹²)₂, —CON(R¹²)₂, —NHC(O)R¹², —NHSO₂R¹², —SO₂N(R¹²)₂ and —CN, or R¹ and R², together with Q and D, combine to form an aryl, hetroaryl, cycloalkyl or heterocycloalkyl ring, any of which can be optionally substituted with up to 4 substituents, which can be the same or different, and are selected from halo, —OH, alkyl, haloalkyl, —O-alkyl, —O-haloalkyl, —NO₂, —CO₂R¹², —N(R¹²)₂, —CON(R¹²)₂, —NHC(O)R¹², —NHSO₂R¹², —SO₂N(R¹²)₂ and —CN, such that R² is absent when D is nitrogen and the optional and additional bond is present;
R³ is heterocycloalkyl or heteroaryl, each of which can be optionally substituted with up to 3 groups, each independently selected from alkyl, —N(R²⁰)₂ or —OR²⁰; and
each occurrence of R²⁰ is independently H or alkyl;
or a pharmaceutically acceptable salt thereof.

55. The compound of claim 54, wherein Y is —C(O)— and Z is alkylene.

56. The compound of claim 54, wherein R³ is:

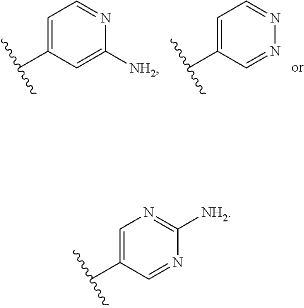

57. The compound of claim 54, wherein Y is —C(O)—, Z is —CH₂— and R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂.

58. The compound of claim 54, wherein the ring: 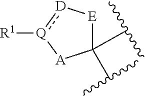
is: 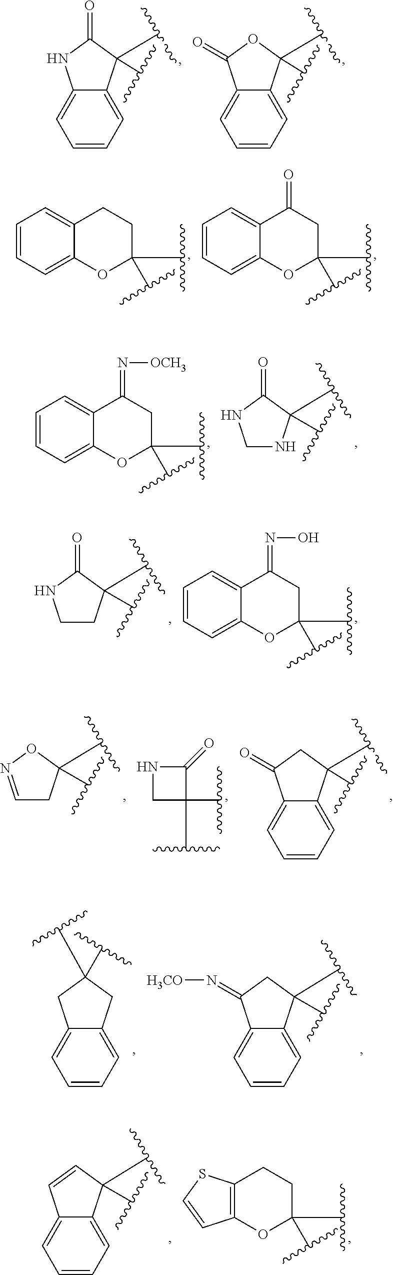 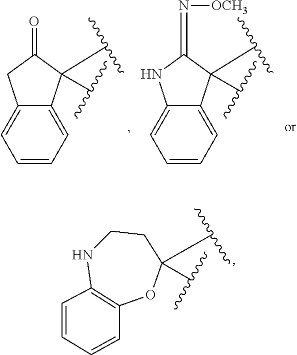
each of which can be optionally substituted with up to 3 substituents, each independently selected from alkyl, phenyl, —NHC(O)—R²¹, halo, benzyl, —NHS(O)₂-alkyl or —NHC(O)N(R²²)₂, wherein R²¹ is alkyl, heteroaryl or haloalkyl, and R²² is H or alkyl.

59. The compound of claim 54, wherein Y is —C(O)—; Z is —CH₂—; R³ is pyridyl, pyrimidinyl, pyridazinyl, tetrahydropyranyl, isoxazolyl, thiazolyl, piperidinyl, pyrazolyl, oxetanyl, azetidinyl, oxazolyl, isothiazolyl, furanyl, each of which can be optionally substituted with up to 3 groups selected from alkyl, —OH or —NH₂.

60. A compound which is selected from the group consisting of: 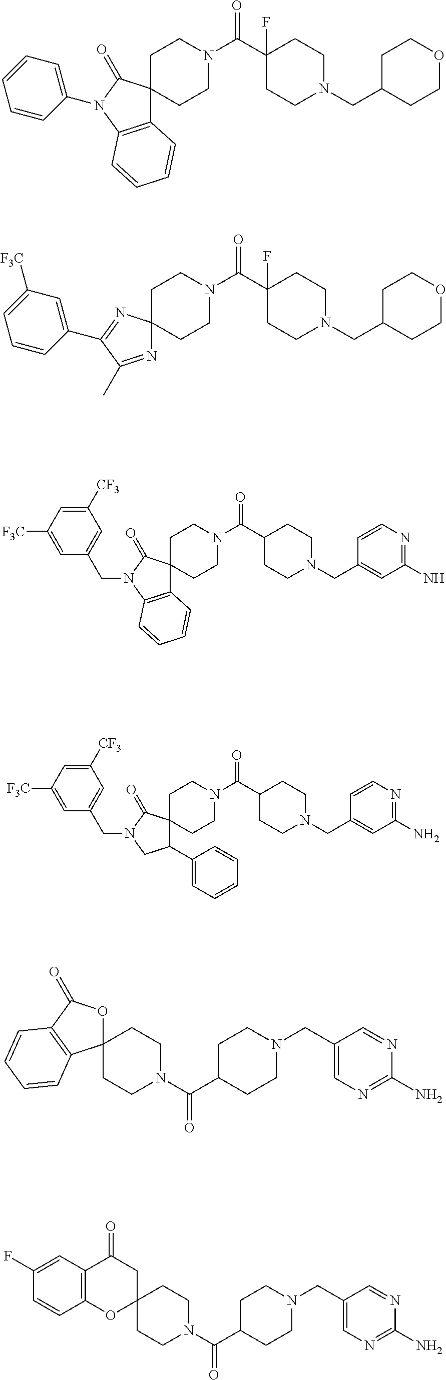 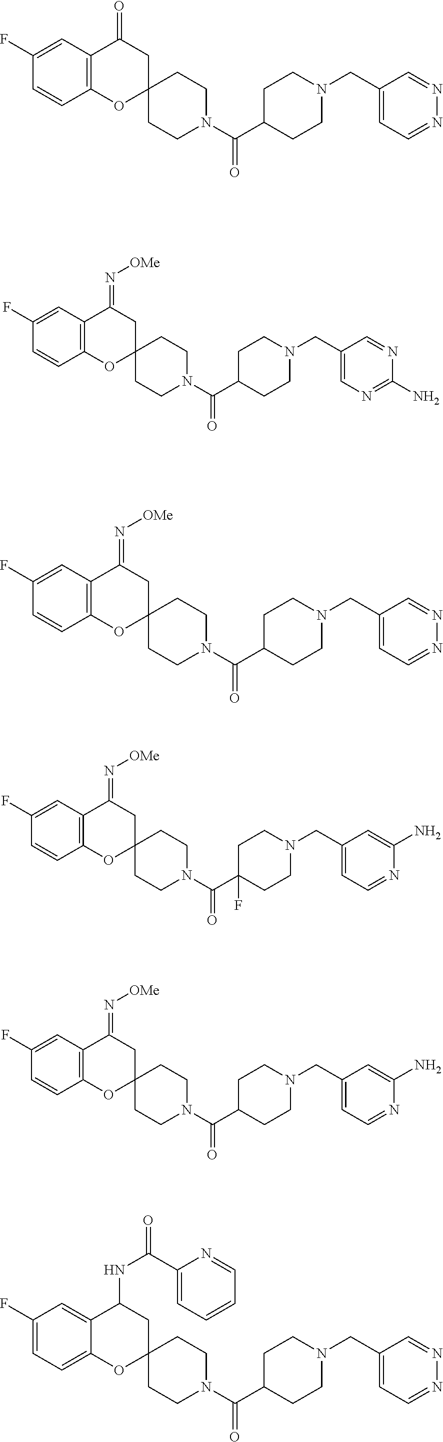 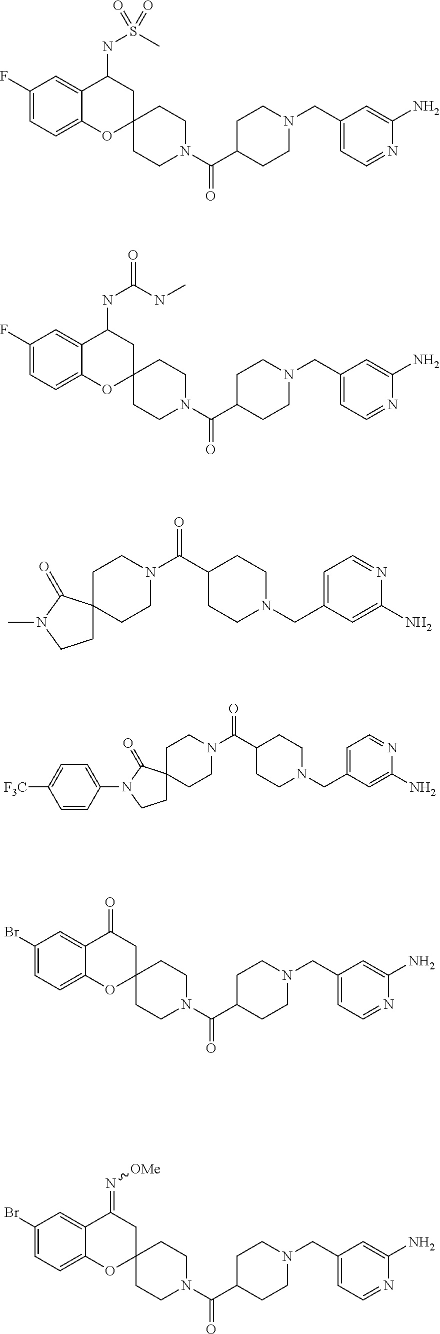 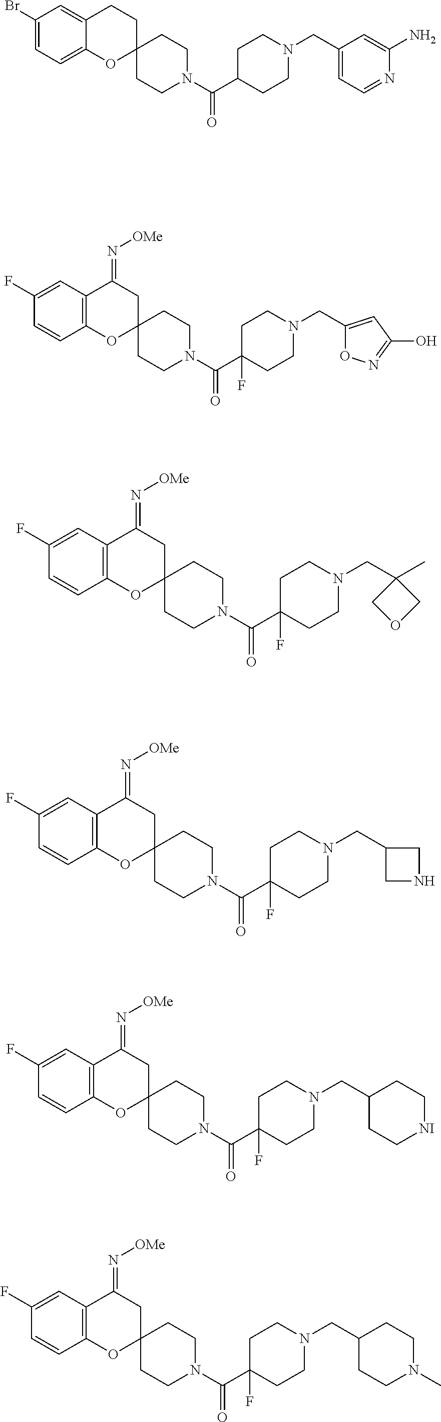 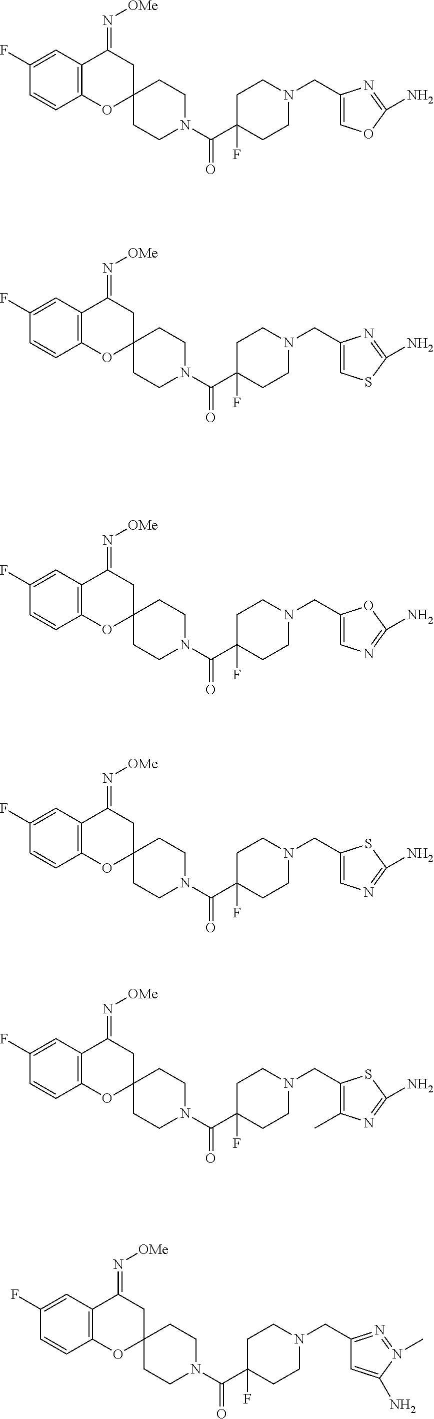 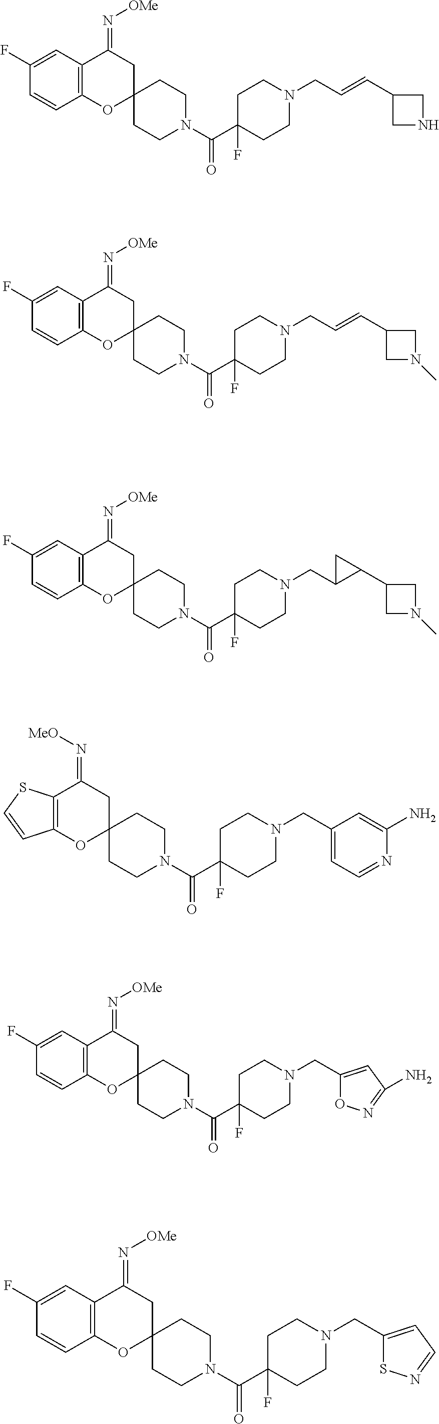 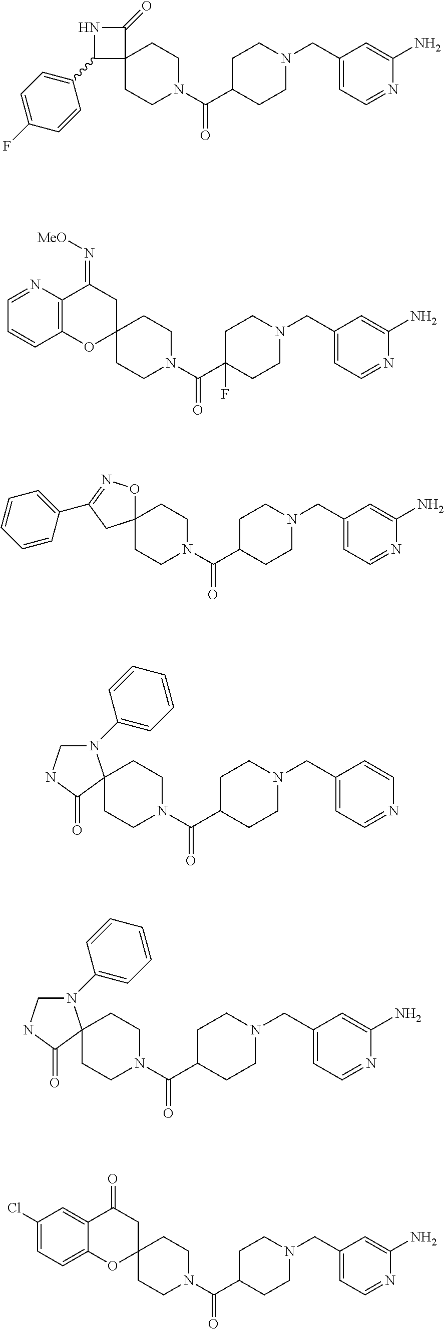 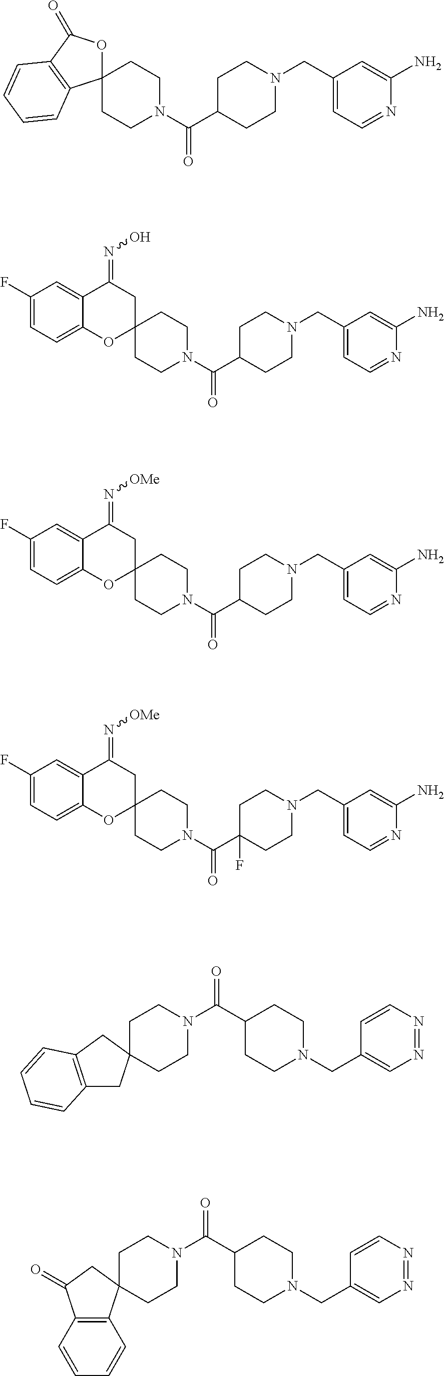 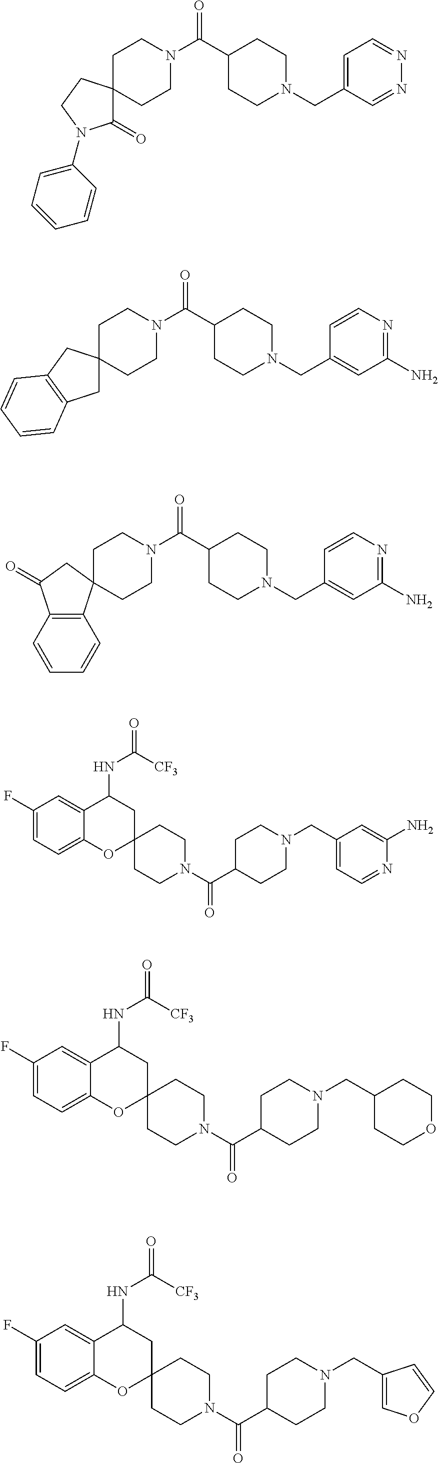 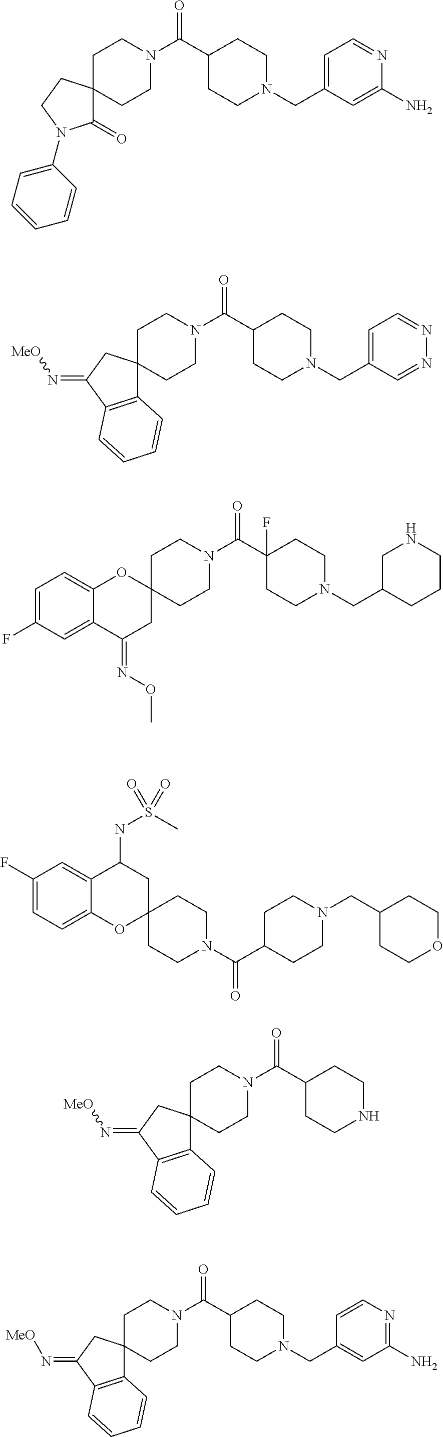 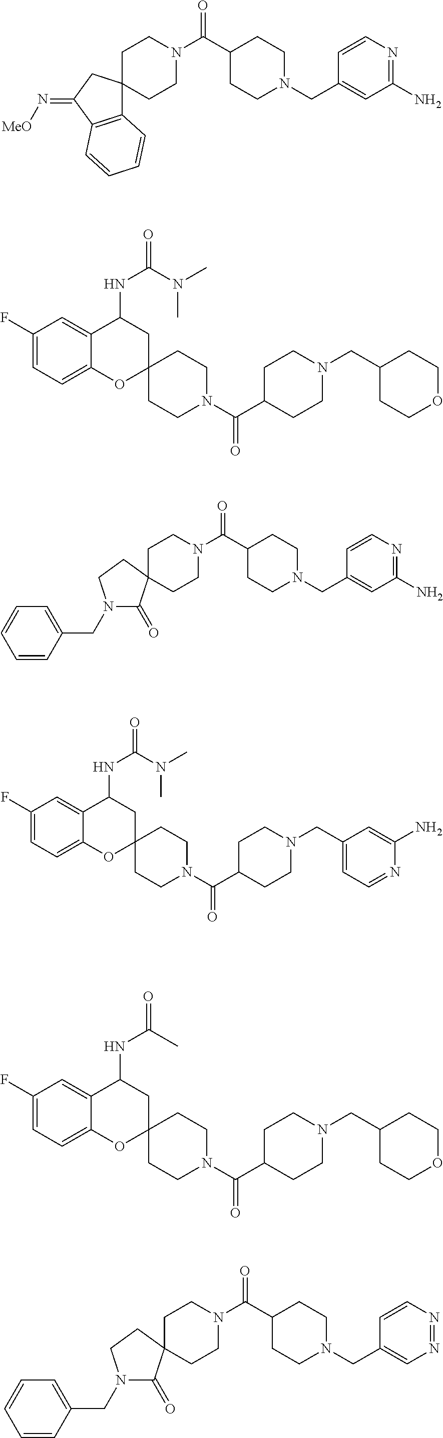 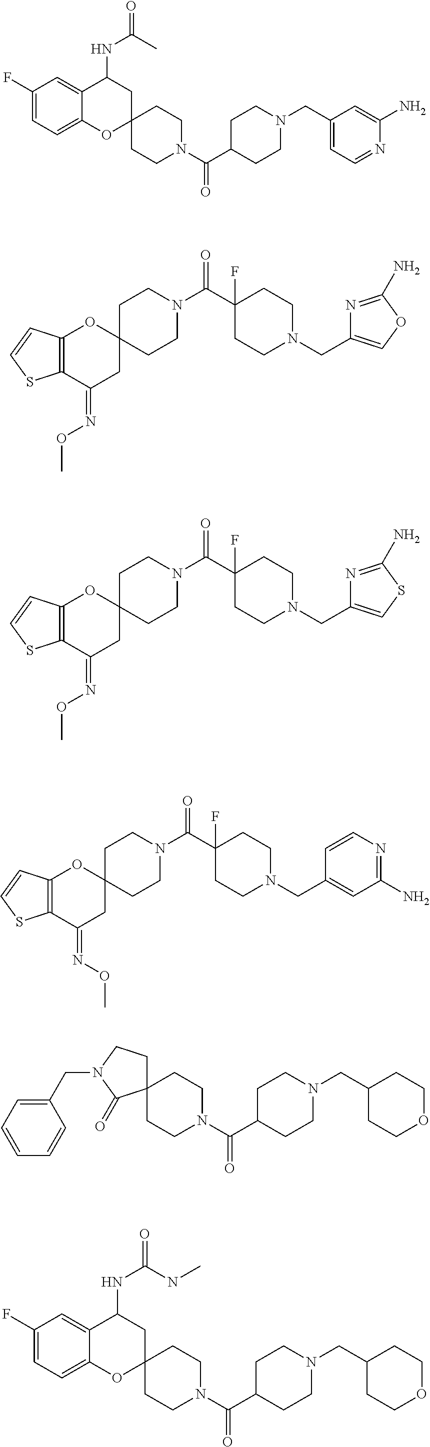 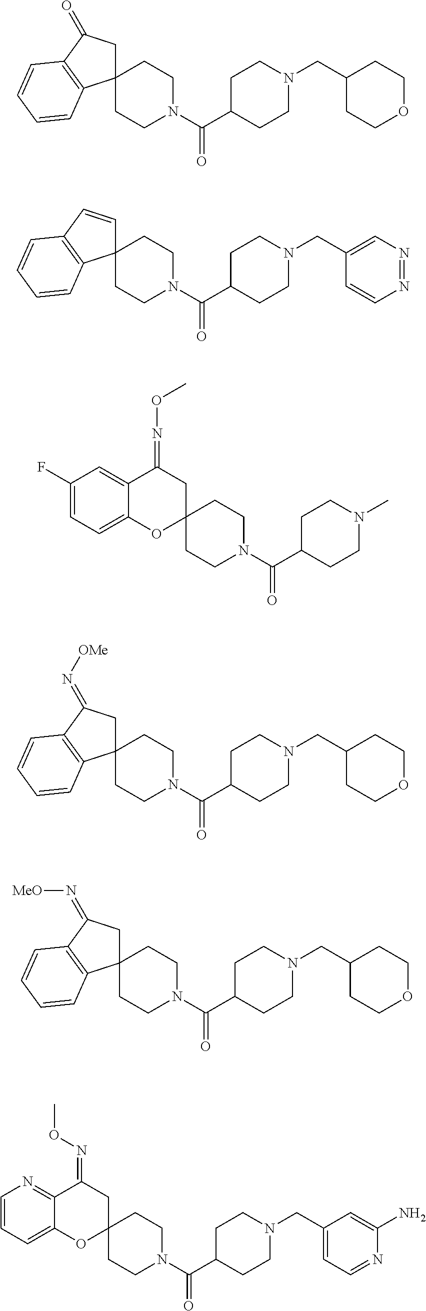 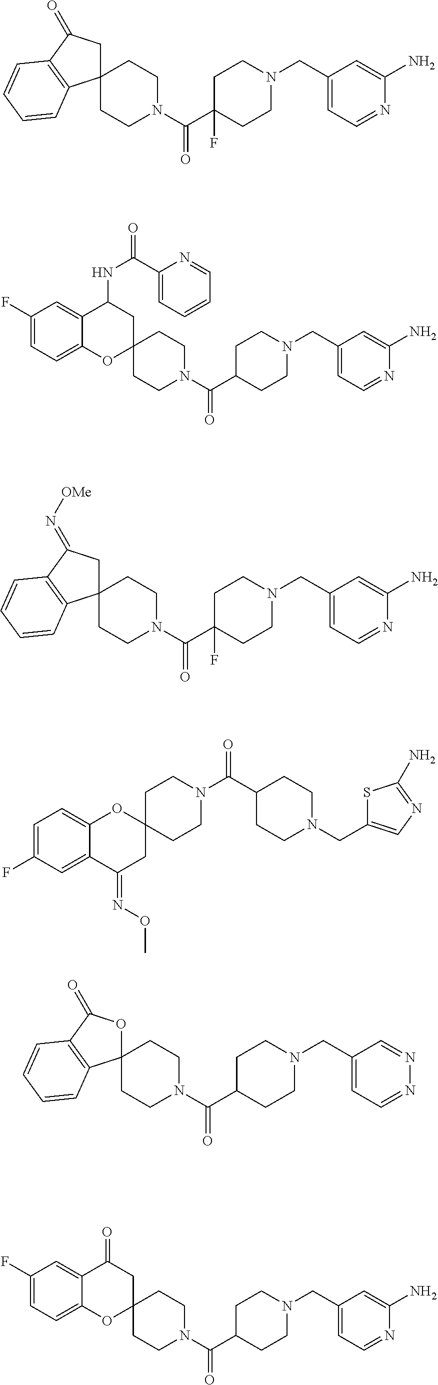 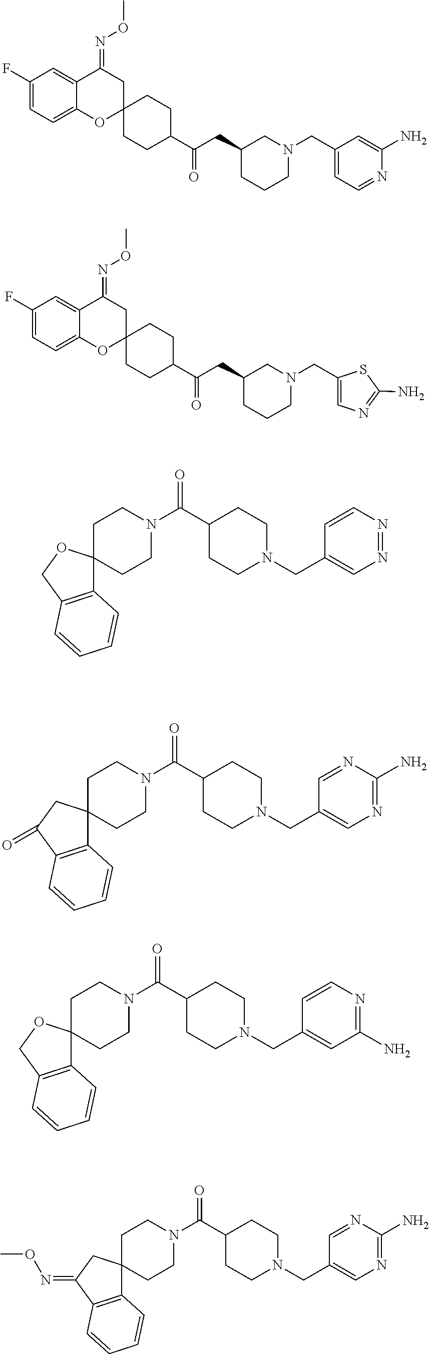 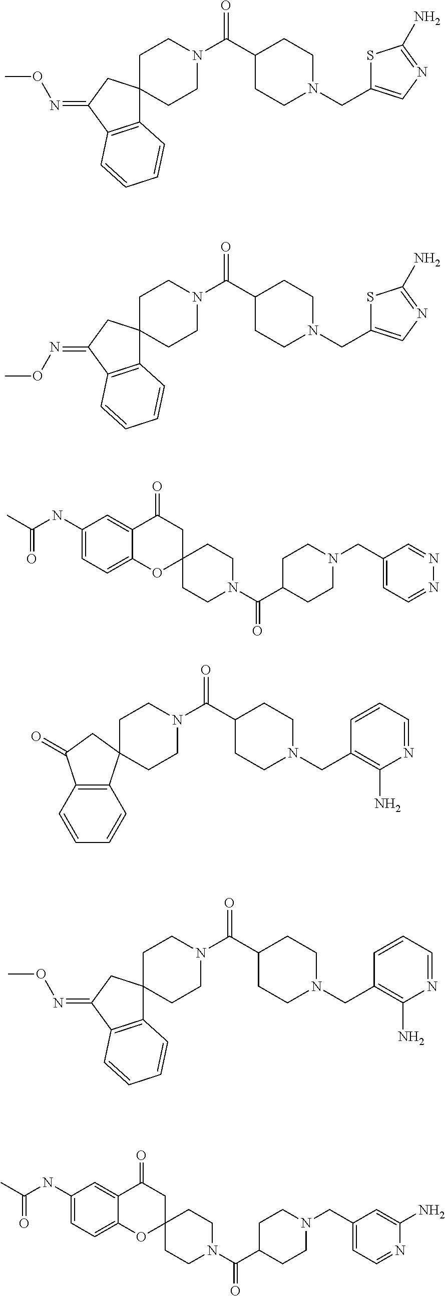 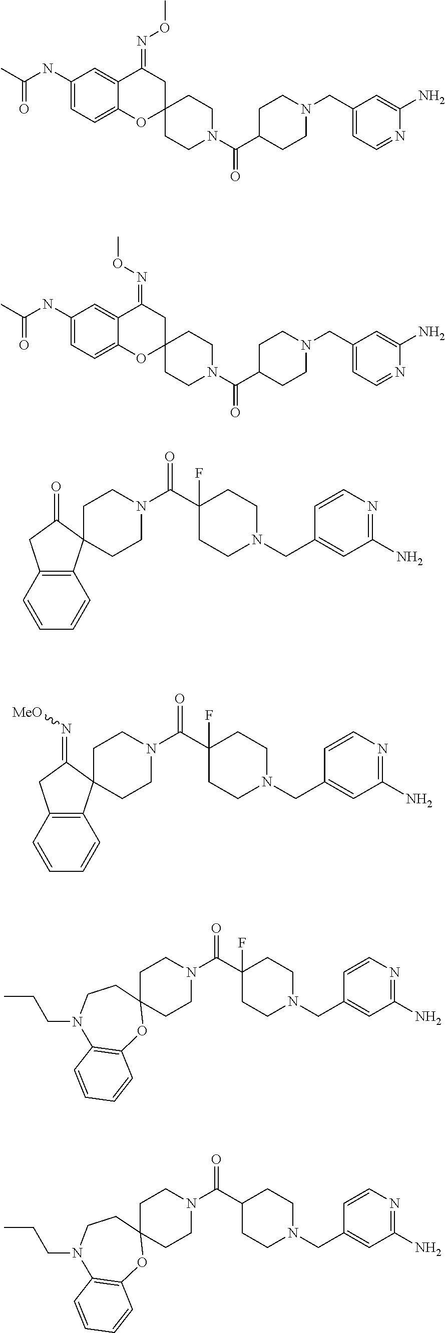
and
or a pharmaceutically acceptable salt thereof.

61. A pharmaceutical composition comprising the compound of claim 53 or a pharmaceutically acceptable salt thereof and a pharmaceutically acceptable carrier.

62. A method of treating allergy, an allergy-induced airway response, congestion, a cardiovascular disease, an inflammatory disease, a gastrointestinal disorder, a neurological disorder, a metabolic disorder, obesity or an obesity-related disorder, diabetes, a diabetic complication, impaired glucose tolerance or impaired fasting glucose in a patient, comprising administering to the patient an effective amount of the compound of claim 54 or a pharmaceutically acceptable salt thereof.
